(19) United States
(12) Patent Application Publication
(10) Pub. No.: US 2005/0130116 A1
(43) Pub. Date: Jun. 16, 2005

(54) Stable expression of polymorphous forms of human cytochrome p450 2d6

(76) Inventors: Johannes Dohmer, Grafelfing (DE); Niels Krebsfanger, Biberach a.d. Riss (DE); Michel Fichelbaum, Murr (DE); Ulrich M. Zanger, Korntal-Munchingen (DE)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/018,320

(22) PCT Filed: Feb. 15, 2001
(86) PCT No.: PCT/DE01/00597

(30) Foreign Application Priority Data
Mar. 14, 2000 (DE) 100 12 220.5

(51) Int. Cl. C12Q 1/00; C12N 5/06

(52) U.S. Cl. 435/4; 435/358

(57) ABSTRACT

The present invention relates to a test system containing cell lines expressing human cytochrome P450 2D6 as well as to the use of said test system for the study of pharmacological and toxicological aspects of the hCYP2D6 polymorphism. The present invention further relates to methods for the detection of novel polymorphic forms of human cytochrome P450 2D6 using the test system according to the invention as well as to methods for the simple and exact quantification of the cytochrome P450 content using CO difference spectra.

The present invention relates to a test system containing cell lines expressing human cytochrome P450 2D6 as well as to the use of this test system for the study of pharmacological and toxicological aspects of the hCYP2D6 polymorphism. Furthermore, the present invention relates to methods for detection of novel polymorphic forms of human cytochrome P450 2D6 using the test system according to the present invention as well as to methods for a simple and exact quantification of the cytochrome P450 content by means of CO difference spectra.

Day by the day, the human body takes up a plurality of foreign substances. Harmful chemicals from the environment, ingredients of food and stimulants, and in some cases also medicaments. All these foreign substances eventually have to be excreted to avoid damages to the organism. Many of these compounds, however, have a poor solubility in water and therefore cannot be excreted easily. Thus, in the course of evolution nearly all living organisms have developed a complex enzyme system capable of converting compounds into a hydrophilic excretable form. This metabolic process is referred to as the metabolism of foreign substances and has been formally divided into two phases (Greim and Deml, 1996; Marquardt and Schäfer, 1997): In phase I the introduction of functional groups into the compound or a demasking of functional groups occurs, a process in which P450 cytochromes play a key role. In phase II the functionalized metabolite is conjugated to substances with good solubility in water, such as sulfates, sugars, glutathione, carboxylic acids, or amino acids. Thereby, the compound is rendered sufficiently hydrophilic to be excretable in the form of a non-reactive final product via the kidney or the intestine. There is a risk, however, that reactive metabolites may react with the body's own structures such as DNA, RNA, proteins, and lipids and induce cytotoxic, cancerogenous or mutagenic effects. In this case, the detoxification is converted into a toxification.

Drugs are metabolized and excreted by the same enzymatic system, a fact which may have an influence on the pharmacological efficacy in different ways: either the metabolites have a lower pharmacological effectiveness as the starting compounds or they may be completely ineffective, as in the case of barbiturates. In other cases, the mother substance as well as the metabolites have an effect. An example are the cough medicine codeine and its metabolite morphine. In other cases, only the metabolite is effective for example the cleavage product of cyclophosphamnide in chemotherapy.

The efficacy of a drug may vary between different individual due to differences in its metabolization. The metabolization is affected by parameters such as age, sex, physical condition, and diet. Differences in the genes encoding foreign substance-metabolizing enzymes such as cytochrome P450 have been shown to be another important factor. Thus, due to genetic differences some individuals can metabolize drugs faster, slower, in a different manner or not at all. If the allelic sequences vary between different populations this will result in interethnical differences in the frequency of phenotypes. Asians, Caucasians and Black Africans will therefore react very different to the same medicament. These pharmacologically important inter-individual differences have been referred to as pharmacogenetic polymorphism (Meyer, 1991):

“A pharmaco-genetic polymorphism is a monogenic feature caused by the presence within a population of more than one allele in the same locus and of more than one phenotype with respect to the efficacy of a drug in the organism. The frequency of the rarest allele is >1%.”

It has been agreed to set the frequency of the rarest allele to >1% since not every base difference will necessarily be relevant for a certain population group. It has been estimated that up to 20% of all medicaments are subject to pharmaco-genetic polymorphism (Blech, 1999). In the USA, more than 2 million people per year suffer from undesirable drug effects which take a lethal course in more than 100,000 cases. Thus, they are among the six most frequent causes of death (Lazarou et al., 1998). As a consequence, “personalized drug(s) (dosages)” are desired which can be specifically adapted to the individual pharmaco-genetics of a particular patient. For example, this is the current practice in the therapy of children suffering from leukemia using azathioprine or 6-mercaptourine which otherwise would lead to life-threatening side effects in 3% of the cases.

Therefore, besides the development of efficient methods for detecting the individual pharmaco-genetic profile such as by DNA chip technology for maximal drug safety it will be required to gain a detailed knowledge of the drug metabolizing enzymes and their genetic polymorphisms.

The most important group of foreign substance metabolizing phase I enzymes are the cytochromes P450 (EC 1.14.14.1; unspecific monooxygenases). In 1958, they were described for the first time idependently by Garfinkel (1958) and Klingenberg (1958) as “cell-coloring pigment” of the liver. They were referred to as cytochrome P450 because cytochromes in their reduced state and after gassing with carbon monoxide have difference spectra showing a characteristic absorption peak at 450 nm. A quantification may be carried out using the absorption together with the molar extinction coefficient (Omura and Sato, 1964a; Omura and Sato, 1964b).

At present, the cytochrome P450 superfamily includes 481 isoforms in 85 different eukaryotic and 20 prokaryotic species (Nelson et al., 1996). The classification of cytochrome P450 enzymes is per conventionem based on their amino acid sequence homologies. Their identity is more than 40% within a family and at least 55% within a subfamily. Cytochrome P450 genes are abbreviated by “CYP” in italics while cDNAs, mRNAs, and proteins are abbreviated by “CYP” followed by an arabic numeral referring to the family as well as a latin capital letter for each subfamily. Individual isoenzymes are numbered chronologically by another arabic numeral. The whole symbol is preceded by a small latin letter indicating the species. Thus, for example human cytochrome P450 2D6 is designated by hCYP2D6 for the gene and hCYP2D6 for cDNA, mRNA, and protein, respectively. All foreign substance metabolizing P450 cytochromes are anchored in the endoplasmatic reticulum as well as in the nuclear membrane by means of a hydrophobic N-terminal sequence and are oriented towards the cytoplasm (Monier et al., 1988). P450 cytochromes belong to the group of heme thiolate enzymes catalyzing the NADPH-dependent monooxygenation of their substrates with formation of the corresponding alcohols or epoxides as well as O- and N-dealkylations. They form a multi-enzyme complex together with NADPH-dependent cytochrome P450 oxido-reductase (CYPOR) and cytochrome b₅ catalyzing the transfer of electrons from NADPH to cytochrome P450. Differences in the ability of complex formation have been observed (Schenkman and Greim, 1993). The activities of some isoforms such as hCYP3A4 is specifically dependent on CYPOR and cytochrome b₅ (Buters et. al, 1994). In the case of hCYP3A4 cytochrome b₅ additionally increases the affinity to certain substrates (Schenkman et al., 1989).

The substrate specificity of individual P450 cytochromes is generally low and often overlapping thereby ensuring sufficient flexibility to deal with the enormous diversity of foreign substances to be metabolized. On the other hand, besides organ-specific expression, polymorphism, and inducibility of many isoenzymes the overlapping substrate specificities substantially contribute to the complexity of the cytochrome P450-catalyzed metabolism of foreign substances and drugs, repectively. The complexity may be further enhanced if the drug metabolism is affected by induction or inhibition of particular cytochrome P450 isoforms during simultaneous administration of several drugs. Therefore, it is required to gain detailed knowledge about drug metabolizing P450 cytochromes on a genetic, regulatory and enzymatic level to avoid undesirable effects caused by metabolism.

Cytochrome P450 2D6 (EC 1.14.14.1; debrisoquine 4-hydroxylase) is one of the molecular species of cytochrome P450 characterized by a marked polymorphism. In humans, CYP2D6 is the only functional isoenzyme of the 2D subfamily, and it was the first cytochrome P450 enzyme for which a genetic polymorphism has been described. By the end of the seventies, the polymorphism of hCYP2D6 has been discovered independently for the antihypertensive drug debrisoquine (Evans et al., 1980; Mahgoub et al., 1977) and the antiarrhythmic drug sparteine (Eichelbaum et al., 1979a; Eichelbaum et al., 1979b). In the next years, the polymorphic metabolic phenotype common to both substrates was demonstrated (Eichelbaum et al., 1982), and its genetic cause was discovered (Daly, 1995; Gonzalez et al., 1988a; Meyer and Zanger, 1997; Price-Evans, 1993; Steiner et al., 1985; Zanger et al., 1988). Today, a plurality of important substrates (cf. Table 1) and 17 alleles are known. Thus, at present the “debrisoquine/sparteine” polymorphism is the most extensive pharmaco-genetic polymorphism which has the highest impact in practise (Bertilsson, 1995; Brosen and Gram, 1989b; Eichelbaum and Gross, 1990; Kroemer and Eichelbaum, 1995; Nebert, 1997; Tucker, 1998).

The expression of hCYP2D6 primarily occurs in the liver in a constitutive manner, and the enzyme is not inducible in contrast to all other drug metabolizing P450 cytochromes 1A1/2, 2B6, 2C8, 2C9, 2C18, 2C19, 2E1 and 3A4/5. During pregnancy, however, a slightly elevated metabolism of hCYP2D6 substrates was observed (Hogstedt et al., 1983; Wadelius et al., 1997). The fraction of the total hepatic cytochrome P450 content is only about 2% and thus relatively low as compared to the two other important drug metabolizing isoforms hCYP3A4 with ≧30% and hCYP2C9 with ≧20% (Shimada et al., 1994). There are dramatic inter-individual differences in the level of expression up to complete deficiency (Shimada et al., 1994).

In addition, an about 100 times lower expression of hCYP2D6 compared to the liver was detected in various extrahepatic tissues, and an association with different diseases has been discussed, in part controversially: in lung/lung cancer (Guidice et al., 1997; Kivisto et al., 1997), in brain/Parkinson (Fonne-Fister et al., 1987; Nebert and McKinnon, 1994; Sabbagh et al., 1999), in the gastrointestinal tract (Prueksaritanont et al., 1995), in breast and in mammary tumors (Huang et al., 1996; Huang et al., 1997), in the bladder mucosa and in tumor tissue (Romkes-Sparks et al., 1994), as well as in peripheral mononuclear blood cells (Carcillo et al., 1996). Of particular interest is the expression in brain since hCYP2D6 metabolizes several pharmaceutics having a central-nervous activity and hydroxylates endogenous tryptamine to give the neurotransmitter dopamine (Hiroi et al., 1998).

hCYP2D6 is involved in phase I metabolism of about 30% of all clinically relevant drugs of different drug groups (cf. Table 1; Alvan, 1991; Brosen and Gram, 1989a; Dahl and Bertilsson, 1993; Eichelbaum and Gross, 1992). Thus, besides hCYP3A4 (55%) and hCYP2C9 (15%) it belongs to the most important drug metabolizing P450 cytochromes despite of its lower level of expression (Smith et al., 1998). For example, the antihypertensive drugs debrisoquine and propafenone, the B blocker propanolol, the tricyclic antidepressant imipramine, etc. have been described as specific substrates of CYP2D6 (Eichelbaum and Gross, 1990). hCYP2D6 is selectively inhibited by quinidine or by specific inhibitory antibodies.

TABLE 1
Examples of drugs and other substrates at least partially metabolized by hCYP2D6.

| Drug Group | Example | Reaction | Reference |
|---|---|---|---|
| monoamineoxidase inhibitors | amiflamine | NDem | (Alvan et al., 1984) |
| β blockers | bufuralol | alH, arH | (Boobis et al., 1985) |
| analgetics | codeine | ODem | (Mortimer et al., 1990) |
| antihypertensives | debrisoquine | arH | (Mahgoub et al., 1977) |
| anorectics | dexfenfluramine | NDea | (Gross et al., 1996) |
| neuroleptics | haloperidol | NDea | (Tyndale et al., 1991b) |
| tricyclic antidepressants | imipramine | arH | (Brosen et al., 1986) |
| α₁ adrenoceptor antagonists | indoramine | arH | (Pierce et al., 1987) |
| β₂ adrenergic stimulants | methoxyphenamine | arH, NDem | (Roy et al., 1985) |
| SSRI | paroxetine | Dem | (Bloomer et al., 1992) |
| antianginal | perhexiline | alH | (Cooper et al., 1987) |
| antidiabetics | phenformine | arH | (Oates et al., 1982) |
| antiarrhythmics | sparteine | H | (Eichelbaum et al., 1979b) |
| anti-estrogenic compounds | tamoxifen | arH | (Dehal und Kupfer, 1997) |
| amphetamine (life-style drug) | MDMA (ecstasy) | alH | (Tucker et al., 1994) |
| endogenous neurotransmitter | tryptamine | arH | (Hiroi et al., 1998) |

Abbreviations:
alH: aliphatic hydroxylation; arH: aromatic hydroxylation; Dem: demethylation; MDMA: methylenedioxymethamphetamine; NDea: N-dealkylation; NDem: N-demethylation; ODem: O-demethylation; SSRI: selective serotonine reuptake inhibitor

According to estimations of the WHO, 250,000 women worldwide die each year because of breast cancer (Logan, 1975). In the USA and Western Europe, breast cancer is the cause of death in 4% of all cases in women (American Cancer Society). In Germany alone about 42,000 women each year fall ill with breast cancer (Becker and Warendorf, 1981-1990). Approx. 30% of the tumors are hormone-sensitive.

The non-steroidal selective estrogen receptor modulator tamoxifen (Novaldex®) is used for the treatment of estrogen-sensitive tumors (Furr and Jordan, 1984; Jordan, 1998; Osborne, 1998). Tamoxifen binds to the estrogen receptor and thus blocks the stimulation of proliferation by estrogen binding. In other organs, however, it shows a desired paradox partial estrogen effect in that it for example counteracts osteoporosis (McGregor and Jordan, 1998). Studies to examine the preventive use of tamoxifen in risk groups are presently carried out (Jordan, 1997; Nayfield, 1995).

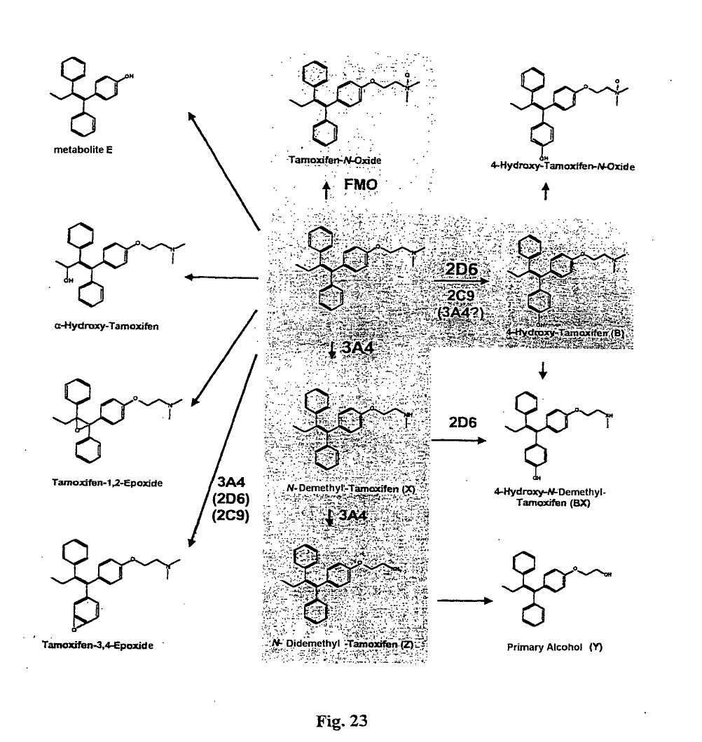

With respect to its pharmacology, metabolite profile, and DNS adduct formation, tamoxifen shows pronounced variations between different species (De Matteis et al., 1998; Glatt et al., 1998; Jordan and Chem, 1982; Jordan and Robinson, 1987; Lim et al., 1994). The main metabolites are N-demethyl-tamoxifen, tamoxifen-N-oxide, and 4-hydroxy-tamoxifen; see FIG. 23. 4-Hydroxy-tamoxifen is about 100 times more potent as an anti-estrogenic substance than the mother substance itself, and thus despite its lower plasma level is believed to contribute substantially to the pharmacological effect (Borgna and Rochefort, 1981; Furr and Jordan, 1984). An involvement in the 4-hydroxylation of tamoxifen is discussed for the cytochrome P450 isoforms hCYP2C9, hCYP2D6, hCYP2E1 and hCYP3A4 (Crewe et al., 1997; Dehal and Kupfer, 1997; Styles et al., 1994). It may be possible that it will be necessary to consider inter-individual differences with respect to the formation of 4-hydroxy-tamoxifen in establishing the therapeutical dose of tamoxifen.

Together with the pseudogenes CYP2D7P and CYP2D8P, CYP2D6 forms a gene cluster in the CYP2D locus at position q13.1 on the long arm of chromosome 22 (Eichelbaum et al, 1987; Gonzalez et al., 1988b; Gough et al., 1993; Kimura et al., 1989). Similar to the two pseudogenes it consists of 9 exons and 8 introns.

Of the 17 known hCYP2D6 alleles only five, namely hCYP2D6*1, hCYP2D6*2, hCYP2D6*9, hCYP2D6*10 and hCYP2D6*17, encode an enzyme with (limited) functionality (Daly et al., 1996a). At least another functional allele is postulated for the population of Ghana (Droll et al., 1998; Masimirembwa et al., 1996a). The phenotypic classification is carried out using the ratio of test substrate/metabolite in urine per time unit. The smaller this “metabolic ratio (MR)”, the faster will be the metabolism of the test substrate, e.g. debrisoquine, dextromethorphane, metoprolol or sparteine. Homozygous carriers of non-functional alleles are always deficient with respect to the 2D6 activity and show a so-called “poor metabolizer (PM)” phenotype characterized by a high MR.

Individuals who are homozygous for the wildtype allele hCYP2D6*1 phenotypically are “extensive metabolizers (EM)” having a low MR (Sachse et al., 1997). Gene amplification of a functional allele results in a so-called “ultrarapid metabolizer (UM)” phenotype (Bertilsson et al., 1993; Johansson et al., 1993; Lundqvist et al., 1999). Homozygous carriers of the allele hCYP2D6*10 showing a limited functionality have an increased MR compared to the EM phenotype and therefore are sometimes referred to as “intermediate metabolizers (IM)” (Armstrong et al., 1994; Yokota et al., 1993).

Furthermore, variations in the allele frequency between different populations result in inter-ethnical differences (Bertilsson, 1995): In Caucasians the frequencies of the functional alleles hCYP2D6*1 and hCYP2D6*2 are about 35% while hCYP2D6*2xN, hCYP2D6*9 and hCYP2D6*10 are rare with frequencies of 1-2% and hCYP2D6*1 7 has not been detected yet. The non-functional alleles hCYP2D6*4 and hCYP2D6*5 have frequencies of about 20% and 5%, respectively, while the frequencies of the other allels is 0-2%. Thus, the proportion of poor metabolizers in the Caucasian population is approx. 5-10% (Alvan et al., 1990; Evans et al., 1993; Griese et al., 1998; Sachse et al., 1997).

In contrast, in the Asian population the frequency for the non-functional allele hCYP2D6*4 is only 0.8% while the allele hCYP2D6*10 with limited functionality occurs in a frequency of 23-70%. Accordingly, only about 1% of Asians are poor metabolizers although their mean MR is increased in comparison to Caucasians (Bertilsson et al., 1992; Dahl et al., 1995b; Horai et al., 1989; Roh et al., 1996).

The same applies to some Black African populations showing allele frequencies of about 4% for hCYP2D6*4, about 5% for hCYP2D6*10 , and 15-35% for hCYP2D6*17 (Evans et al., 1993; Masimirembwa et al., 1996b).

A heterologous expression of several hCYP2D6 alleles has been carried out in various systems: in E. coli (Gillam et al., 1995; Kempf et al., 1995; Pritchard et al., 1998), in yeast (Bichara et al., 1996; Ellis et al., 1992; Krynetski et al., 1993; Krynetski et al., 1995; Oscarson et al., 1997), in insect cells (Evert et al., 1997; Paine et al., 1996; Patten et al., 1996), in CHO cells (Patten et al., 1996), in COS-1 cells (Gonzalez et al., 1990; Johansson et al., 1994; Kagimoto et al., 1990; Oscarson et al., 1997), in Hep G2 cells (Aoyama et al., 1990; Tyndale et al., 1991a), in NIH3T3 cells (de Groene et al., 1996), and in human B lymphoblastoid cells AHH-1 TK± (Crespi et al., 1991; Crespi et al., 1995; Penman et al., 1993). In addition, the wildtype allele hCYP2D6*1 has been already expressed in V79 Chinese hamster cells (Fischer et al., 1992; Rauschenbach et al., 1997).

Nevertheless, up to now no test system exists which is suitable for an examination of the pharmacological, toxicological, and other aspects of the hCYP2D6 polymorphism.

The technical object underlying the present invention is to provide a test system enabling a comparative examination of the metabolic activity of active forms of human cytochrome P450 2D6 with regard to a wide variety of substances. Preferably, this system is intended to be suitable as an analytical tool in preclinical drug development and for in vitro analysis of the human metabolism of foreign substances in comparison to that of other species. The system shall contribute to a replacement and completion of animal experiments in pharmacology and toxicology. Particularly, the system is intended to be suitable for phase I studies of drug metabolism as well as to enable a reliable, simple and cost-effective identification of the enzymes involved in the metabolism of a candidate drug at a time point as early as possible in preclinical drug development.

Another technical object underlying the present invention is to provide methods for the study of pharmacological and toxicological aspects of hCYP2D6 polymorphism. Preferably, these methods are contemplated for a use in the preclinical phase of drug development, they shall be carried out under standardized and reproducible conditions and are intended to have a high predictive value for humans. Preferably, these methods are intended to replace animal experiments in this phase.

A method for the determination of the cellular content of cytochrome P450 by means of CO difference spectra is well-known. According to this method, 10¹⁰ cells are required to record CO difference spectra in a cellular system. This amount of cells can only be achieved with relatively high effort using special cell culture techniques such as cultivation on microcarriers while with an extinction.difference between 450 nm and 490 nm of about 0.001 the resulting spectra are still unsatisfactory (Onderwater et al., 1996). Therefore, the amount of heterologously expressed cytochrome P450 has been often estimated by means of Western analyses (e.g. Wolfel et al., 1991; Schneider et al., 1996). This method, however, can only detect the content of cytochrome apoprotein while it is important to determine the holoenzyme as the functionally active cytochrome P450 including the prosthetic heme group.

Thus, another object underlying the present invention is to provide a method for the simple, sensitive and exact quantification of the cytochrome P450 content, particularly in cellular expression systems.

These objects are solved by the subject-matter of the claims.

In its first aspect, the present invention relates to a test system comprising cell lines each expressing different functional human cytochrome P450 2D6 alleles in a heterologous manner. The test system contains three or more of said cell lines. The cytochrome P450 2D6 alleles expressed may be selected with respect to the frequency of their presence in a population to be tested. For example, if the most frequent alleles in a population are hCYP2D6 alleles *1, *10, and *17, the test system according to the present invention contains three cell lines each expressing one of said alleles. In a preferred embodiment, the five hCYP2D6 alleles *1, *2, *9, *10, and *17 encoding functional enzymes will be heterologously expressed in a cellular system. A preferred cell for expression generally lacks any cytochrome P450 activity. The level of expression and the enzyme kinetic characteristics of the system expressing recombinant hCYP2D6 according to the present invention preferably are similar to both the physiological situation and to comparable expression systems. A preferred expression system are eukaryotic cells, in particular mammalian cells, and most conveniently fibroblast cells. In one embodiment the cells are derived from Chinese hamster and preferably are Chinese hamster lung fibroblasts or cells derived therefrom, particularly V79 cells. In a preferred embodiment, cDNA is expressed. Preferably, the test system according to the present invention is suitable for a comprehensive in vitro analysis of the human cytochrome P450 2D6 polymorphism. In a particularly preferred embodiment, the test system enables testing and comparing the properties of the five known functional forms of human cytochrome P450 2D6 under standardized experimental conditions. According to the present invention it is preferred to employ parental and mock-transfected cell lines, respectively, as the negative controls.

In the fifties, the cell line V79 was estasblished from morphologically and neoplastically transformed lung fibroblasts of an adult male Chinese hamster. The establishment of the V79 cell line has not been published. From the description of the experimental series for establishing cell lines of Chinese hamster lung tissue it is possible to infer the establishment of the V79 cell line by analogy (Ford and Yerganian, 1958). Since then it has been widely used in toxicity and mutagenicity studies (Bradley et al., 1981; Chu and Malling, 1968; Doehmer, 1993; Sawada and Kamatki, 1998; Swierenga et al., 1991), and has been certified according to OECD Guidelines for the Testing of Chemicals.

The V79 cell lines employed in the test system according to the present invention may be already established V79 cell lines.

Some of the known V79 cell lines, however, show marked differences among each other. Subclone V79MZ is particularly preferred. In contrast to other V79 cells, this subclone grows adherent to a substrate and and is not released. Furthermore, in contrast to V79NH cells, for example, this subclone has no acetyltransferase activity which might affect the measurements conducted with the test system according to the present invention. Therefore, the test system according to the present invention in V79MZ cells bears significant advantages.

Preferred cytpchrome P450 2D6 expressing cells according to present invention therefore are cell lines V79MZh2D6*1, V79MZh2D6*2, V79MZh2D6*9, V79MZh2D6*10 and V79MZh2D6*17 deposited on Feb. 15, 2000, at the DSMZ-Deutsche Sammlung von Mikroorganismen und Zellkulturen GmbH under the accession numbers DSM ACC2446, DSM ACC2447, DSM ACC2448, DSM ACC2449 and DSM ACC2450.

By providing the hCYP2C expressing test system of the present invention, there has been practically provided the completely human phase I drug metabolism in the form of an in vitro test system. The cell lines according to the present invention are suitable for the identification of metabolically competent cytochrome P450 isoforms for a given substrate, for the detection of metabolite profiles and substrate binding mechanisms, for an examination of enzyme kinetics and drug interactions as well as for cytotoxicity and genotoxicity studies.

The test system according to the present invention in eukaryotic cells is suitable for metabolic studies of functional human cytochrome P450 2D6 isoforms. In addition, it avoids the ethical problems associated with the use of organ material of human or animal origin. Moreover, the test system has the advantage that the experimental conditions are exactly defined and may be standardized which cannot be achieved in the case of in vivo systems or native tissue samples due to the extraordinary complexity and inter-individual variability of these materials. Thus, in complex systems the identification of metabolically competent P450cytochromes can only be performed in an indirect manner, for example by means of inhibition by inhibitory antibodies or by using chemicals with inhibitory effects following an induction of the isoform to be studied by chemicals such as dexamethasone, phenobarbital or dioxin. An indirect identification has only an indicative value since inhibitory antibodies rarely achieve the complete inhibition or have an insufficient specificity. Chemicals with inhibitory effects are not specific for cytochrome P450 at all. Inducing chemicals may be misleading since a slight stimulation of other P450 cytochromes may mask the involvement of the cytochrome P450 which is induced in the first place. In contrast, the heterologous expression according to the present invention enables a direct and unambiguous assignation of cytochrome P450 isoforms and metabolites.

The test system according to the present invention has the advantage in contrast to studies using purified enzyme that the cells may be used without laborious purification steps. In addition, the system according to the present invention avoids an alteration of the substrate specificity of the cytochrome P450 2D6 forms tested or a contamination of the enzymes to be tested during a purification. Moreover, the system in mammalian cells has the advantage as compared to e.g. yeast cells that it provides relevant and important biological and metabolic endpoints as well as a metabolic competence which are comparable to those of an animal or of man. Particularly, the system according to the present invention in V79 cells has the advantage that these cells provide a plurality of toxicological endpoints with a low and stable background and therefore are especially suitable for mutagenicity and toxicity studies (Bradley et al., 1981). In contrast to all other mammalian cells including human cell lines V79 cells are particularly characterized by an extraordinarily stable pseudodiploid karyotype having a constant chromosome number which can be maintained also after transfection with foreign DNA (Doepker et al., 1998; Simi et al., 1999). This is important regarding cytogenetic target points. Furthermore, the stability of cells is important for a reliable examination according to the methods of the present invention. With less than 12 h the doubling time of V79 cells is the shortest compared to all other cell lines studied up to now. Most importantly, V79 cells do not express any endogenous cytochrome P450 (Kiefer and Wiebel, 1989; Onderwater et al., 1996). Particularly for cell line V79MZ it was shown that no endogenous cytochrome P450 is expressed, and thus the cells are exactly defined for the cytochrome P450 isoenzyme transfected. Moreover, it has been shown for cell line V79MZ that the cells exhibit adherent growth and show a stable phenotype in culture.

Furthermore, endogenous heme as well as cytochrome b₅ are synthesized in sufficient amounts in V79 cells (Onderwater et al., 1996). In contrast, in high-expressing systems such as baculovirus infected insect cells the endogenous heme synthesis is insufficient for a saturation of the expressed cytochrome with prosthetic heme (Asseffa et al., 1989; Barnes et al., 1994; Buters et al., 1994; Paine et al., 1996). In the system according to the present invention using V79 cells a supplementation with or coexpression of cytochrome P450 NADPH oxido-reductase is necessary only in special cases (Schneider et al., 1996). According to the present invention, however, there are also comprised test systems containing cell lines which coexpress functional forms of human cytochrome P450 2D6 and a cytochrome P450 NADPH oxido-reductase and/or cytochrome b₅, preferably of human origin. The system according to the present invention has the further advantage that it does not require adaption of the nucleic acid sequences, particularly cDNA sequences, as in the case of expression in E. coli (Sengstag et al., 1994). Moreover, suitable intracellular membrane systems for an incorporation of cytochrome P450 are present so that no subsequent reconstitution is required (Gillam et al., 1995). In contrast to yeast cells, for example, V79 cells are also permeable for many substrates. Therefore, it is also possible to perform metabolic studies by utilizing the cell's own metabolism, e.g. for the regeneration of NADPH, directly in cell culture.

According to the present invention there is provided a kit containing the test system of the present invention of cell lines expressing human cytochrome P450 2D6. The kit according to the invention may contain the cell lines expressing human cytochrome P450 2D6 according to the present invention and/or a lysate and/or a microsomal fraction thereof and/or a fraction enriched in human cytochrome P450 2D6 and/or purified human cytochrome P450 2D6. As further components, the kit according to the invention may comprise e.g. growth media, control substances and cells, liver extract, liver homogenate and/or liver microsomes and/or metabolically active cells such as primary hepatocyte cultures and/or metabolically active enzymes such as cytochrome P450, etc.

In the following there will be described the different possibilities of use of the test system containing human cytochrome P450 2D6 expressing cells according to the present invention. The methods of the present invention are either carried out with whole cells, a lysate, or a microsomal fraction thereof, a fraction enriched in cytochrome P450 2D6, or with cytochrome P450 2D6 purified from cytochrome P450 2D6 expressing cells according to the present invention. In one embodiment, the cells of the present invention are contemplated for a use in combination with liver extract, homogenate and/or microsomes and/or metabolically active cells, such as primary hepatocyte cultures and/or metabolically active enzymes such as other P450 cytochromes, carboxylesterases, exoxide hydrolases, flavin containing monooxigenases, N-acetyltransferases, sulfotransferases, glutathione-S-transferase, methyltransferases, monoamino and diamino oxidases, etc.

In addition, the cells may be used in a combination with electrodes for cultivation on silicon or similar materials for a direct coupling of enzyme and data media and may be employed as metabolically competent Bio-Chips in pharmacology and toxicology.

The test system according to the present invention of human cytochrome P450 2D6 expressing cell lines may be employed in the study of gene-dependent toxicity of certain metabolites such as drugs. Furthermore, the system of the present invention enables the examination and determination of the metabolic activation of cancerogenous substances and thus enables the determination of the cancerogenous effect of specific compounds, particularly depending on the form of human cytochrome P450 2D6 expressed. Thus, the present invention relates to a method for the identification of mutagenic, cancerogenous, or toxic effects of substances wherein the cell lines expressing human cytochrome P450 2D6 according to the present invention are contacted with the substance to be tested. For example, a mutagenic effect may be detected by a measurement of cells resistant to certain cell toxins, such as antibiotics, or cells with altered metabolism such as the appearance of a hypoxanthine guanine phosphoribosyl transferase (HGPRT) negative phenotype. A toxic effect of the substance to be tested may be for example detected by a decrease in viability of the cell lines of the invention. A cancerogenous effect may be examined by the appearance of a malign proliferation phenotype of the cell lines of the present invention, such as unlimited division, or due to the capability of the cell lines of the present invention to generate tumors in test animals. Thereby, the test system according to the present invention provides a high experimental sensitivity and enables the examination of the metabolic functions of different polymorphic forms of functional human cytochrome P450 2D6.

The suitability of the system of cell lines according to the present invention as an analytical tool in preclinical drug development has been demonstrated according to the invention using the pharmacologically and toxicologically important 4-hydroxylation of the breast cancer therapeutic tamoxifen as an example. The correspondence of the catalytic properties of the novel polymorphic cell lines with the results of earlier in vitro and in vivo studies demonstrates the suitability of the novel cell battery for in vitro analysis of hCYP2D6 polymorphism. Using the cell lines according to the present invention it will be possible for the first time to perform comparative studies of enzyme kinetics for the different polymorphic human hCYP2D6 forms. The cell lines of the invention are particularly suitable for the identification of cytochrome P450 isoforms involved in complex metabolic situations and for the determination of their metabolic profiles because they are exactly defined for individual isoforms and allow for working under standardized, reproducible conditions. In addition, in accordance with the results obtained from the hCYP2D6 specific hydroxylation of bufuralol it could be demonstrated according to the present invention that carriers of alleles hCYP2D6*17 and particularly of hCYP2D6*10 developed markedly lower plasma levels of 4-hydroxy-tamoxifen than carriers of the other functional alleles. Because 4-hydroxy tamoxifen is at least 100 times more potent as an anti-estrogenic substance than tamoxifen itself, individual differences in the formation of this active metabolite may have a substantial clinical impact on the therapeutic effect.

Thus, another aspect of the present invention relates to a method of preclinical drug development using the test system according to the present invention. For this purpose, in vitro studies shall indicate hCYP2D6 polymorphism-dependent differences in the in vivo metabolism of different compounds including drugs such as tamoxifen. Presumably, marked inter-individual differences in metabolic processing which lead to the formation of pharmacologically less active or more potent metabolites such as the formation of 4-hydroxy tamoxifen from tamoxifen must be considered in establishing the therapeutical dose.

Furthermore, the test system according to the present invention enables the identification of individuals who belong to a risk group due to the metabolic activity or deficiency of the specifically expressed forms of cytochrome P450 2D6. Such individuals are for example characterized by a metabolic conversion of xenobiotics into toxic, mutagenic, or cancerogenous forms or by a lack of detoxification of drugs or other xenobiotics.

To measure the 4-hydroxylation of tamoxifen, the cells of the test system according to the present invention or e.g. a homogenate thereof are reacted with tamoxifen. The reaction product, 4-hydroxy-tamoxifen, may then be determined using well-known methods. Differences in the amount of reaction product between the different cell lines of the test system of the present invention demonstrate whether tamoxifen is metabolized, poorly metabolized or not metabolized at all by the different forms of human cytochrome P450 2D6.

According to the present invention there is provided a method for drug screening using the test system according to the present invention of cytochrome P450 2D6 expressing cell lines. By using the method it will be possible to find substances which are metabolized or not metabolized by the various forms or by specific forms of human cytochrome P450 2D6. For this purpose, a substance to be tested is contacted successively with the different cell lines of the test system of the present invention, and a metabolic product is measured. The presence of a metabolic product indicates that the respective form of human cytochrome P450 2D6 is capable of metabolizing the substance. Thus, with this method according to the present invention it is possible to find new drugs which are derivatives of already known compounds and keep a pharmacological effect while being less metabolized or not metabolized at all. Moreover, this method enables the discovery of substances which show better metabolization and thereby ensure a faster detoxification of the body.

For example, different modified forms of tamoxifen which preferably have a pharmacological activity may be contacted with the cells of the test system according to the present invention or e.g. a homogenate thereof and may be reacted therewith. The amount of specific metabolites being the reaction products, such as a 4-hydroxylated form, indicates whether a modified form of tamoxifen is metabolized well or poorly by the different or by specific forms of human cytochrome P450 2D6.

Another aspect of the present invention relates to a method for the detection of novel alleles of hCYP2D6. According to this aspect of the present invention the heterologous expression of an allele in question is carried out. A preferred cell for expression generally lacks any cytochrome P450 activity. The level of expression and the enzyme kinetic properties of the expression system preferably correspond to both the physiological situation and to that of similar expression systems. A preferred expression system are eukaryotic cells, particularly mammalian cells, and most conveniently fibroblast cells. In one embodiment the cells are derived form Chinese hamster, and preferably are lung fibroblast cells of the Chinese hamster oder derived therefrom, particularly V79 cells and more preferably the subclone V79MZ. In a preferred embodiment cDNA is expressed. Subsequently, the cell line expressing the allele in question is tested with respect to the metabolism of one or more compounds including drugs such as tamoxifen and compared to the metabolism of the test system according to the present invention which preferably expresses three to five of the hCYP2D6 alleles *1, *2, *9, *10 and *17. Marked differences in the formation of specific metabolites such as the formation of 4-hydroxy-tamoxifen indicates that the allele in question is a novel hCYP2D6 allele. Then, the allele in question or the novel allele and the expressed gene product may be further analyzed according to known methods including a determination of the nucleic acid sequence and the encoded amino acid sequence. By comparison to the already known hCYP2D6 alleles the novel allele may be further characterized and differences in nucleic acid sequence such as mutations, insertions, or deletions, or in the amino acid sequence such as substitutions, insertions, or deletions may be determined.

Another aspect of the present invention relates to a method for the simple and exact quantification of the cytochrome P450 content, particularly of the hCYP2D6 content by means of CO difference spectra. By solubilization of cytochrome P450 with the non-ionic detergent emulgen 913 and subsequent centrifugation to minimize the turbidity of the solubilisate the sensitivity of the measuring procedure is increased by a factor of 3000 as compared to known measuring procedures on the basis of CO difference spectra.

In a preferred embodiment the quantification of the cytochrome P450 content is carried out in a cellular expression system such as the expression system according to the present invention. The method of quantification of the present invention preferably enables a direct comparison of polymorphic forms of hCYP2D6. Using the method according to the present invention it will be possible to take CO difference spectra in a cellular system while 100 times less cells than before may be used. The exact quantification of cytochrome P450 using the method of the present invention enables a comparison of orthologous or polymorphic isoforms under defined conditions.

Preferably, the method according to the present invention comprises the following steps:
(a) preparation of cell homogenate;
(b) addition of emulgen 913 to the cell homogenate;
(c) removing insoluble material;
(d) determination of the reduced spectrum;
(e) saturation with carbon monoxide;
(f) measurement of the CO/reduced spectrum;
(g) evaluation of the cytochrome P450 content by means of the spectra.

From the two spectra obtained the CO/reduced versus the reduced spectrum (CO difference spectrum) is evaluated as in step (g), and the concentration of cytochrome P450 and cytochrome P420 is derived therefrom.

Preparation of the cell homogenate is preferably performed by shock freezing and disrupting the cells in liquid nitrogen. Preferably, protease inhibitors such as PMSF are added to the cell homogenate. It is preferred to carry out the addition of emulgen 913 in step (b) together with a buffer such as 100 mM sodium hydrogenphosphate, pH 7.4, 10% (v/v) glycerol. In a preferred embodiment emulgen 913 is added in a final concentration of 0.25% (w/v). After addition of emulgen 213 the membrane-bound cytochrome P450 is solubilized preferably by stirring on ice, and the insoluble material in step (c) is removed by centrifugation. Prior to measurement, the suspension without insoluble material may be reduced by sodium dithionite. Preferably, the spectra are recorded between 400 and 500 nm, and extinction coefficients of 91 mM⁻¹ cm⁻¹ and 110 mM⁻¹ cm⁻¹ are used for the calculation of the concentrations of cytochrome P420 and cytochrome P450, respectively.

The abbreviations used herein have the following meanings:

Besides abbreviations used according to Duden, the conventional codes for amino acids and nucleotides as well as the common abbreviations for restriction enzymes, polymerases etc. were used.
A absorption
APS ammoniumpersulfate
bp base pair(s)
BSA bovine serum albumine
cDNA complementary deoxyribonucleic acid
CMV cytomegalovirus
Cyt b₅ cytochrome b₅
Cyt_{c} cytochrome c
CYP cytochrome P450 protein, mRNA, cDNA
CYP cytochrome P450 gene
CYPOR NADPH-dependent cytochrome P450 oxido-reductase
Da Dalton
DEPC diethylpyrocarbonate
DMEM Dulbecco's modified Eagle's medium
DMSO dimethylsulfoxide
DNA deoxyribonucleic acid
dNTP deoxynucleoside triphosphate
ECL enhanced chemiluminescence
E. coli Escherichia coli
EDTA ethylenediaminetetraacetate
EM extensive metabolizer
FCS fetal calf serum
FITC fluoresceine isothiocyanate
G418 geneticin 418 sulfate
H homogenate
HEPES N-(2-hydroxyethyl)-piperazine-N′-2-ethanesulfonic acid
IM intermediate metabolizer
kbp kilo base pairs
kDa kilo Daltons
K_{M} Michaelis Menten constant
LB Luria broth
LMW low molecular weight
LTR long terminal repeat
MPSV myeloproliferative sarcoma virus
MR metabolic ratio
Mᵣ relative molecular weight
NADP⁺ nicotinamide adenine dinucleotide phosphate, oxidized
NADPH+H⁺ nicotinamide adenine dinucleotide phosphate, reduced OD optical density
P pellet
PAGE polyacrylamide gel electrophoresis
PBS phosphate buffered saline without Mg²⁺ and Ca²⁺
PCR polymerase chain reaction
PEG polyethylene glycol
pK_{B} base exponent
PMSF phenylmethylsulfonylfluoride
PM poor metabolizer
RSV rous sarcoma virus
SDS sodiumdodecylsulfate
SV40 simian virus 40
TAM tamoxifen
TEMED N, N, N′, N′-tetramethylenediamine
Tris tris-(hydroxymethyl)-aminomethane
TRITC tetramethyl rhodamine isothiocyanate
TSS transformation storage solution
U unit(s) (enzyme unit(s))
UM ultrarapid metabolizer
rpm rotations per minute
V79 cells V79 Chinesische hamster fibroblasts
V79MZ cells V79 Chinesische hamster fibroblasts, Mainz subclone
V79MZCYP cells genetically engineered V79MZ cells heterologously expressing cytochrome P450
V79MZh2D6 cells genetically engineered V79MZ cells heterologously expressing human cytochrome P450 2D6
Vₘₐₓ maximal reaction rate
% (m/v) weight percent
% (v/v) percent by volume

Abbreviations of species:
b bovine
h human
m murine
r rat
f fish (Stenotonus chrysops)

The invention is further explained with respect to the following drawings:

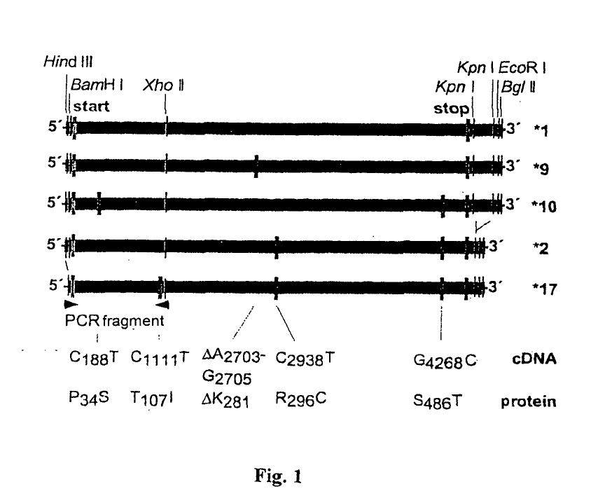

FIG. 1 shows the structure of hCYP2D6 cDNAs expressed according to the present invention. The wildtype cDNA hCYP2D6*1 encodes native hCYP2D6 1 with Val₃₇₄ (GenBank #g181349; SwissProt #P10635; Crespi et al, 1995; Gonzalez et al., 1988). The mutations in cDNAs hCYP2D6*2, *9, *10 and *17 with respect to the wildtype cDNA hCYP2D6*1 are indicated. The positions are numbered according to Kimura et al. (1989).

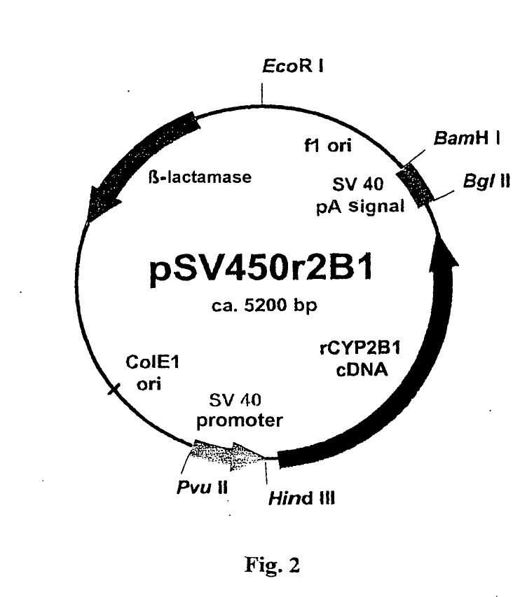

FIG. 2 shows the structure of the original vector pSV450r2B1 (Doehmer et al., 1988). The vector contains the following elements: fragment EcoR I-BamH I: from monkey virus SV40 (GenBank #J02400; Fiers et al. 1978); fragment BamH I-Pvu II: expression cassette which must be stably integrated into the genome of V79 cells; fragment BamH I-Bgl II: contains the early SV40 polyadenylation signal derived from SV40; fragment Bgl II-Hind III: inserted cDNA; fragment Hind lII-Pvu II: contains the early SV40 promoter derived from SV40; fragment Pvu II-EcoR I: from pBR322 (GenBank #J01749).

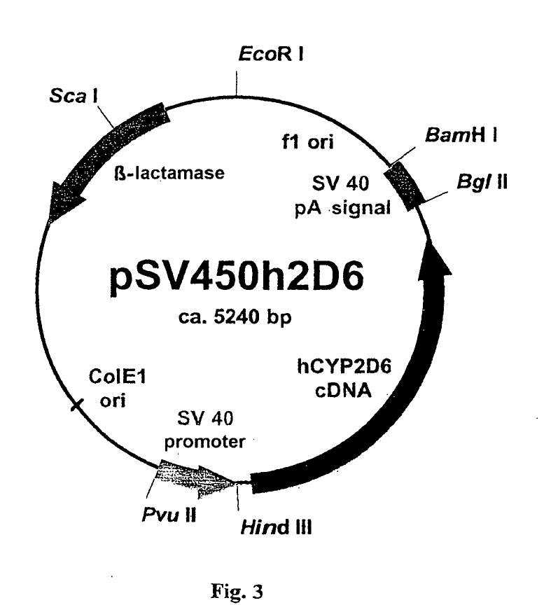

FIG. 3 shows the expression vector pSV450h2D6 for the expression of hCYP2D6 cDNAs: cf. FIG. 1. Expression vector pSV450h2D6 was linearized with Sca I prior to transfection.

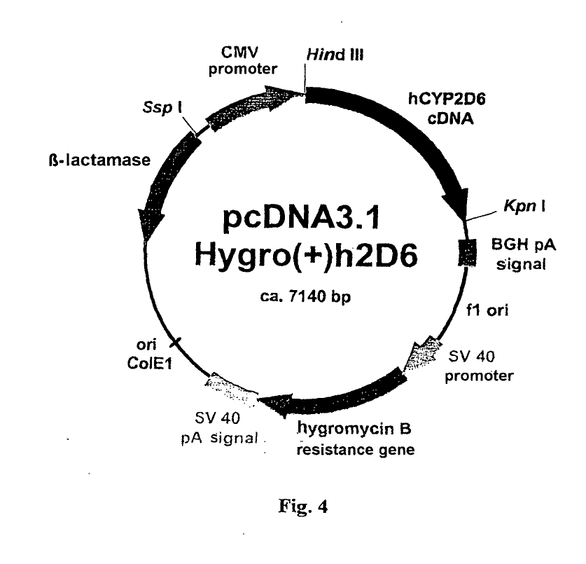

FIG. 4 shows expression vector pcDNA3.1Hygro(+)h2D6. The cDNA is under the control of the cytomegalovirus (CMV) promoter. The hygromycin B resistance gene is regulated via the early SV40 promoter. Both expression cassettes must be stably integrated into the genome of V79 cells. The vector was linearized with Ssp I prior to transfection.

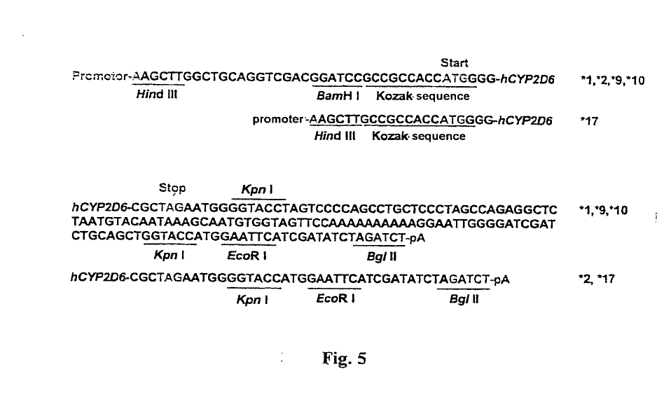

FIG. 5 shows the linker regions between the early SV40 promoter and the hCYP2D6 cDNA as well as between the hCYP2D6 cDNA and the early SV40 polyadenylation sequence in expression vector pSV450h2D6. The Kozak sequence is important for a high rate of translation of the mRNA (Kozak, 1987; Kozak, 1990).

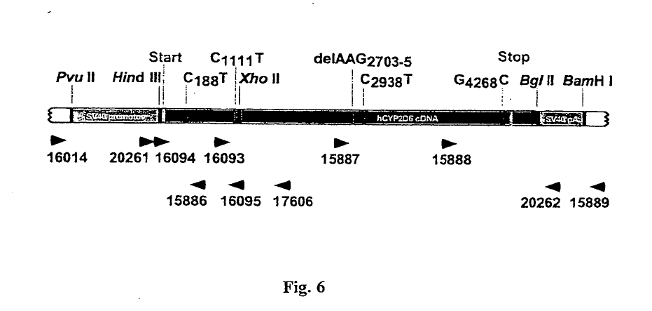

FIG. 6 shows the relative positions and orientations of the primers used in the present invention with respect to plasmid pSV450h2D6. Primers 16298 and 16299 are complementary to the ends of template 16300, primers 19176 and 19177 to the ends of template 19183. Primers 18383 and 18384 are complementary to regions approx. 50 base pairs upstream and 30 base pairs downstream, respectively, of the polylinker of vector pcDNA3.1Hygro(+).

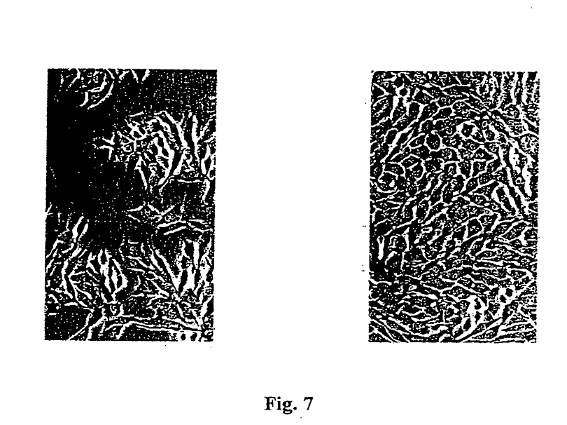

FIG. 7 shows a light micrograph of parental V79MZ cells using phase contrast at an enlargement of ×200 and a confluence of almost 50% (left) and 100% (right). The cells grow as a flat layer adherent to the bottom and have numerous nucleoli. Mitotic cells, in the ideal case about 3% of all cells, have temporary rounded shapes.

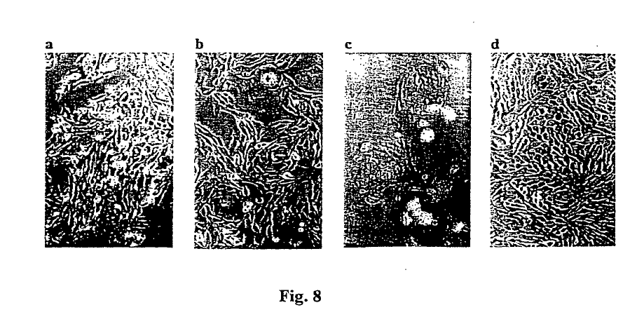

FIG. 8 illustrates the morphological alterations of V79MZ cells due to the transfection with respect to clones V79MZh2D6*9#C6 (a), V79MZf1A1#2 (b) and V79MZh2E1#13 (c) as examples in comparison to parental V79MZ cells (d) in phase contrast at an enlargement of ×200.

Some of the cells were strongly enlarged, rounded and remarkably often polyploid (b and c). A fraction of 2-3% of dead cells (light spots in a, b, and c) was present during the whole cultivation. The doubling time was increased. In some cases the confluence did not exceed approx 70% even during continuous cultivation (b and c). Clones showing morphological alterations were discarded.

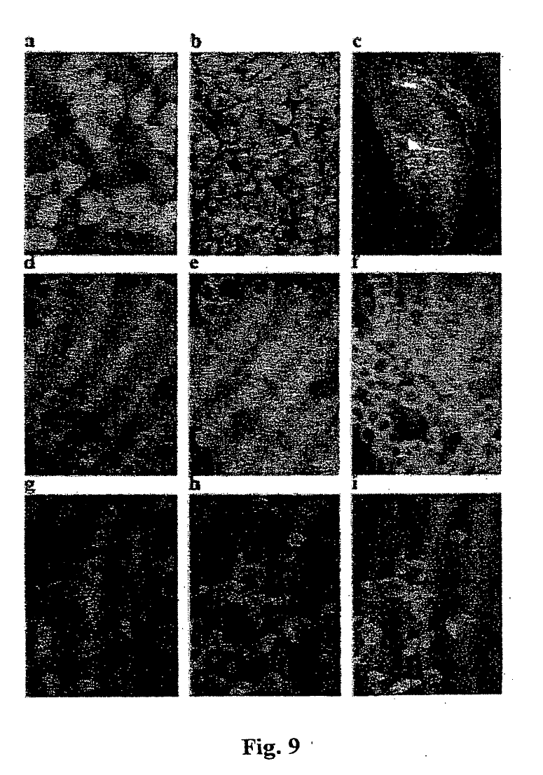

FIG. 9 shows an in situ immunofluorescence micrograph.

a) control mixture: V79MZh2D6*1-S cells show an intense color in contrast to parental V79MZ cells;

b) homogenous clone V79MZh2D6*2;

c) Confocal Laser Scanning Microscopy for detecting the localization of hCYP2D6 in the endoplasmic reticulum;

d-f) double staining of the homogenous clone V79MZh2D6*1-hOR: hCYPOR shows a red color (d), hCYP2D6 1 is stained in green (e). In double exposures of the film the two superimposed colors appear as yellow (f);

g-h) control mixture for double staining: in double exposures V79MZh2D6*1-hOR cells appear yellow (i), V79MZh2D6*1 cells are red, and V79MzhOR cells are green.

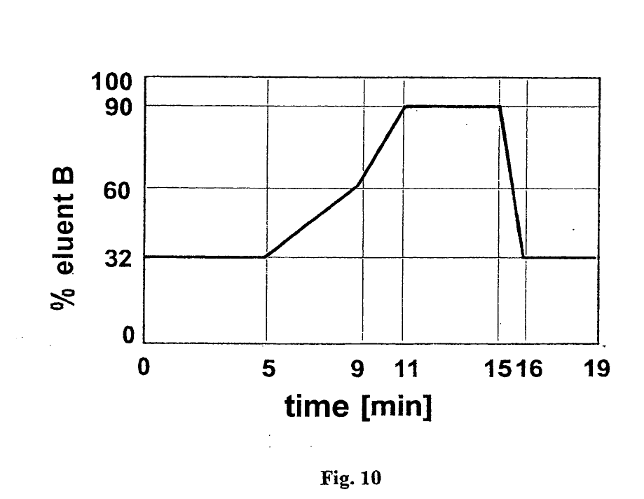

FIG. 10 shows an acetonitrile gradient for the separation of tamoxifen and its metabolites. The eluent A used was water, 1% acetic acid, and the eluent B was acetonitrile, 1% acetic acid.

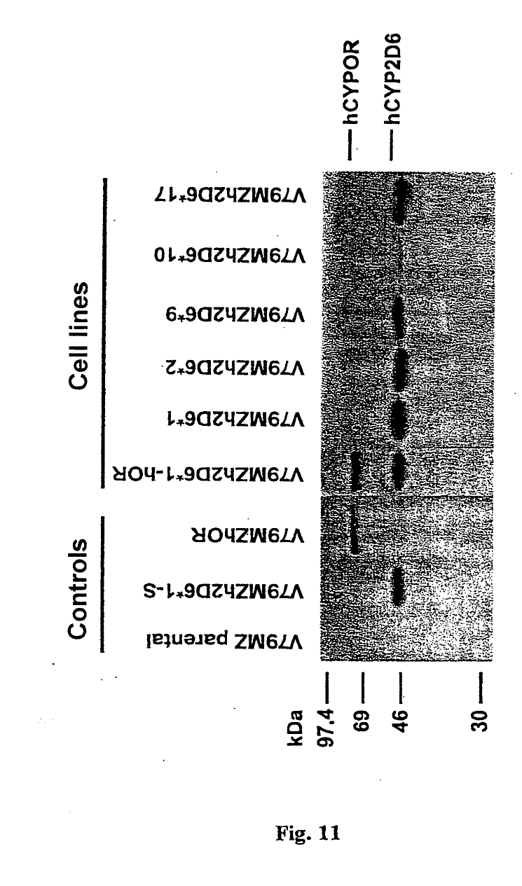

FIG. 11 shows a Western blot of the V79MZh2D6 cell lines according to the invention. 5 μl of each cell homogenate corresponding to 5-15 μg of cellular protein were applied.

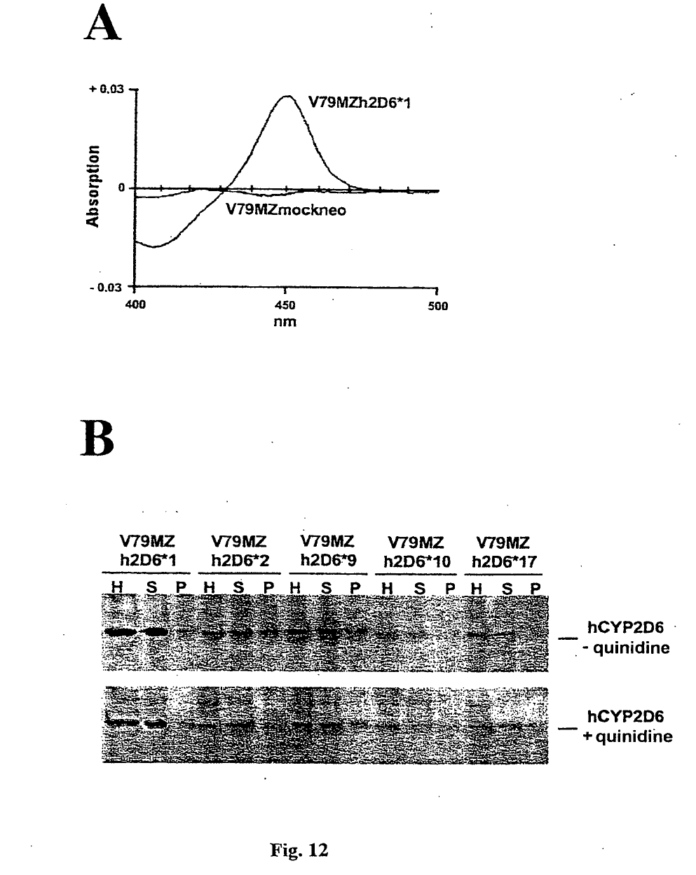

FIG. 12: Solubilization with emulgen 913. A. CO difference spectrum of cell line V79MZh2D6*1 after solubilization in emulgen 913 compared to the negative control V79MZmockneo. The characteristic peak at 450 nm was shifted to blue for about 1.8 nm by the solubilization. B. Western blot to confirm the quantitative solubilization of cytochrome P450. Applied were about 5 μg each of cellular protein of the solubilized cell homogenate prior to centrifugation (H), of the solubilisate after centrifugation (S), and of the pellet resuspended in the same volume (P).

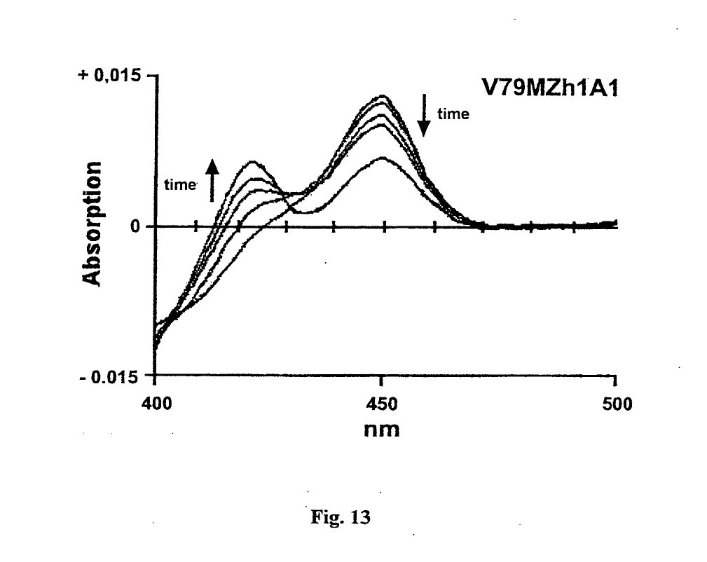

FIG. 13 shows CO difference spectra of cell line V79MZh1A1 taken at 0, 5, 15, 30 and 60 min after saturation of the sample with carbon monoxide. With increasing time cytochrome P450 is degraded to cytochrome P420 by reactive oxygen species.

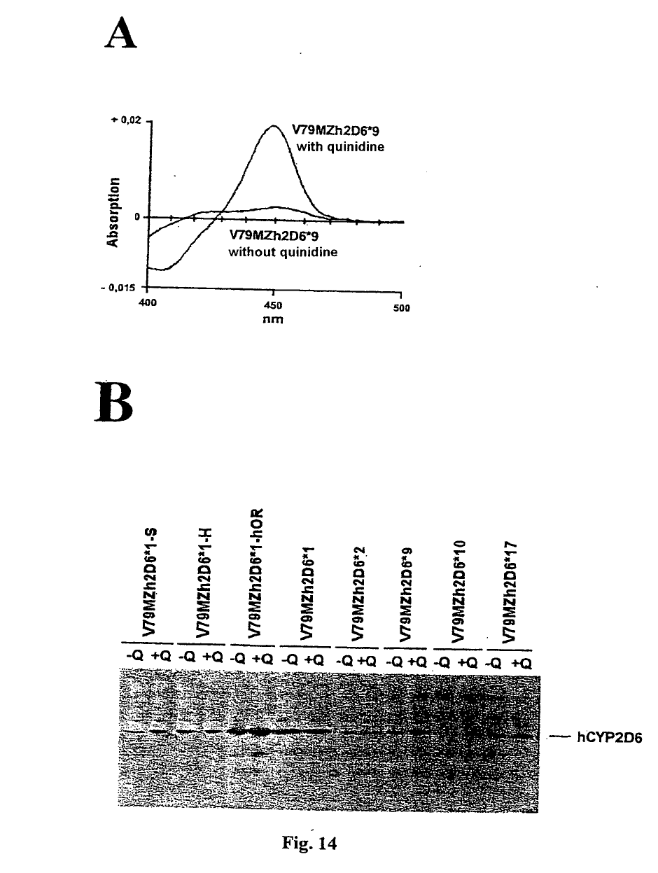

FIG. 14: Effect of quinidine on the stability of hCYP2D6. A. CO difference spectra of cell line V79MZh2D6*9 after cultivation with and without quinidine in culture medium. B. Western blot of solubilized cell homogenate prior to centrifugation. For comparison purposes, 5 μg of cellular protein each of cells grown in culture medium without (−Q) and with quinidine (+Q) were applied.

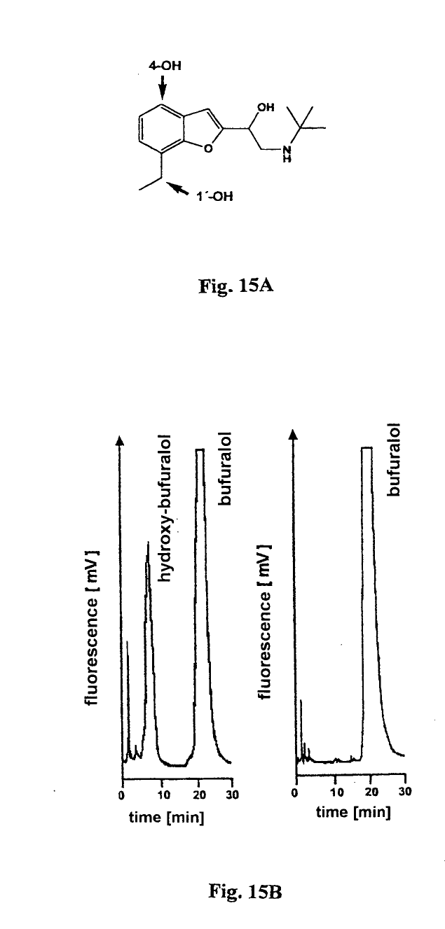

FIG. 15 shows the hydroxylation of bufuralol. A: structural formula of bufuralol. (+)-bufuralol is hydroxylated at C_{1′}, (−)-bufuralol is hydroxylated at C₄. B: HPLC profile after incubation with a homogenate of cell line V79MZh2D6*1 (left) in comparison to the negative control V79MZmockneo (right). The retention time was approx. 8 min for hydroxy-bufuralol and approx. 22 min for bufuralol.

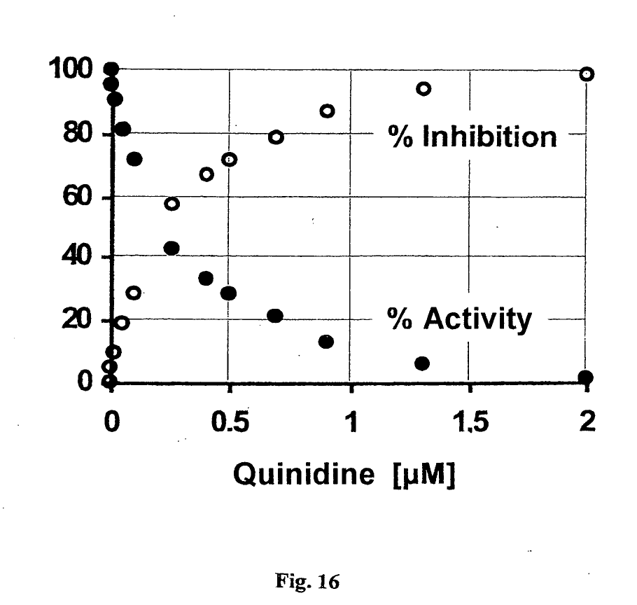

FIG. 16 shows the inhibition of bufuralol hydroxylation by the hCYP2D6-specific inhibitor quinidine using hCYP2D6*1 as an example.

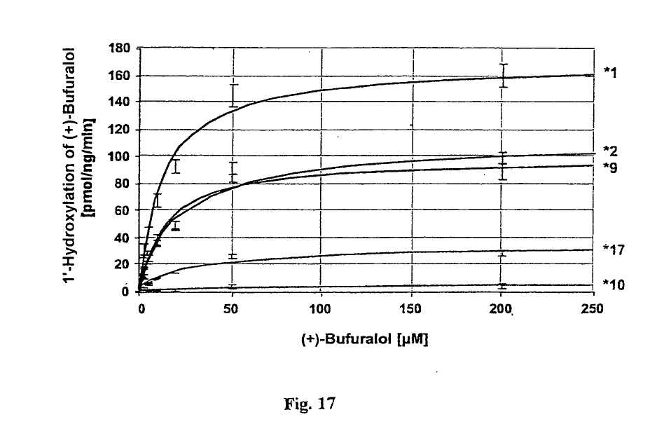

FIG. 17 shows the kinetics of the 1′-hydroxylation of (+)-bufuralol measured with homogenate of the cell lines V79MZh2D6*1, *2, *9, *10 and *17.

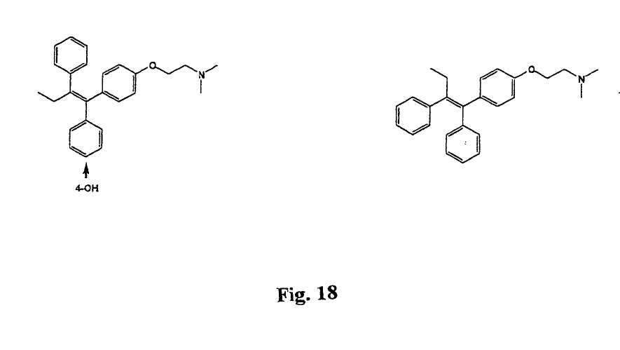

FIG. 18 shows the structural formulas of the clinically administered Z-tamoxifen (left) and its configuration isomer E-tamoxifen (right). The position of the 4-hydroxylation on Z-tamoxifen is indicated.

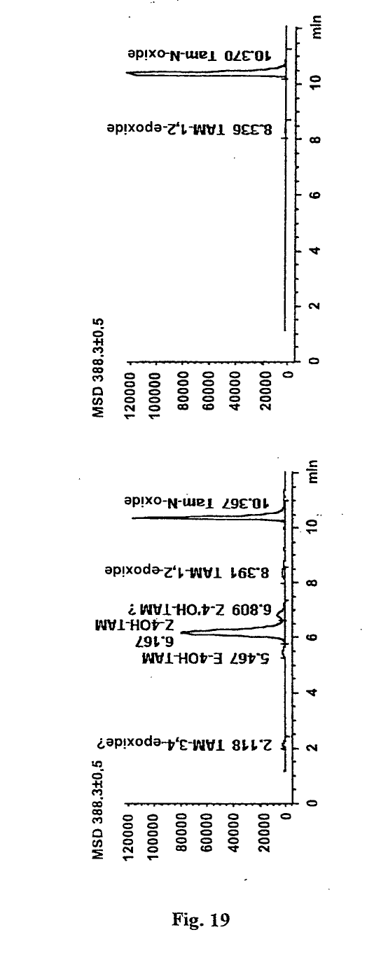

FIG. 19 shows an HPLC/MSD profile after incubation with homogenate of cell line V79MZh2D6*1 (left) in comparison to the negative control V79MZmockneo (right). The retention times were about 5.5 min and 6.2 min for E-tamoxifen and Z-4-hydroxy-tamoxifen, respectively, about 8.4 min for tamoxifen-1,2-epoxide, and about 10.4 min for tamoxifen-N-oxide. The peaks at 2.1 min and 6.8 min could not be assigned unambiguously since no appropriate standards were available.

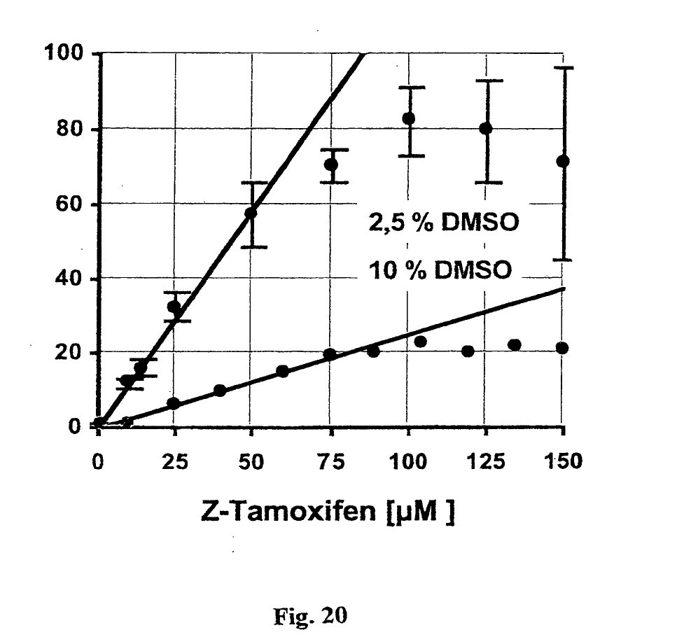

FIG. 20 shows the relationship between the hCYP2D6-catalyzed 4-hydroxylation of tamoxifen and the concentrations of DMSO and tamoxifen. The mean values and standard deviations from three independent measurements (2.5% DMSO) and the mean values of a single measurement series (10% DMSO), respectively, are given.

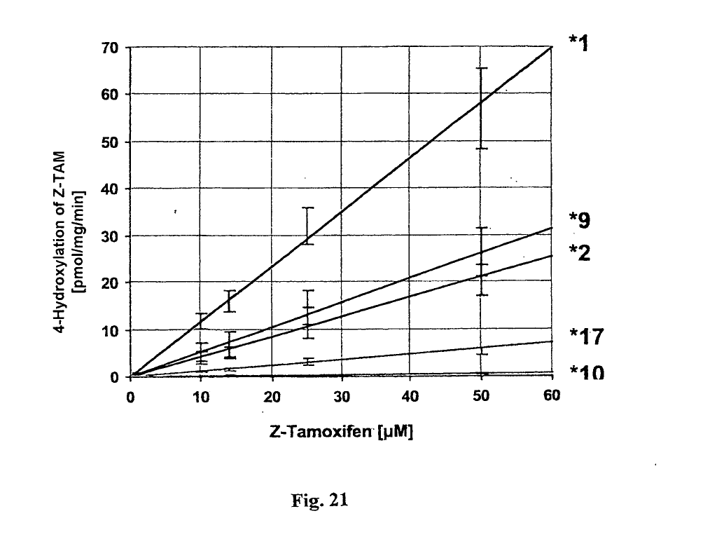

FIG. 21 shows the kinetics of the hCYP2D6-catalyzed 4-hydroxylation of tamoxifen measured with homogenates of cell lines V79MZh2D6*1, *2, *9, *10 and *17 using 2.5% DMSO as solubilizing agent. The onset slope is linear up to the solubility limit of tamoxifen at 50 μM. The slope of the regression line corresponds to Clᵢₙₜ. The mean values and standard deviations from three independent measurements are given.

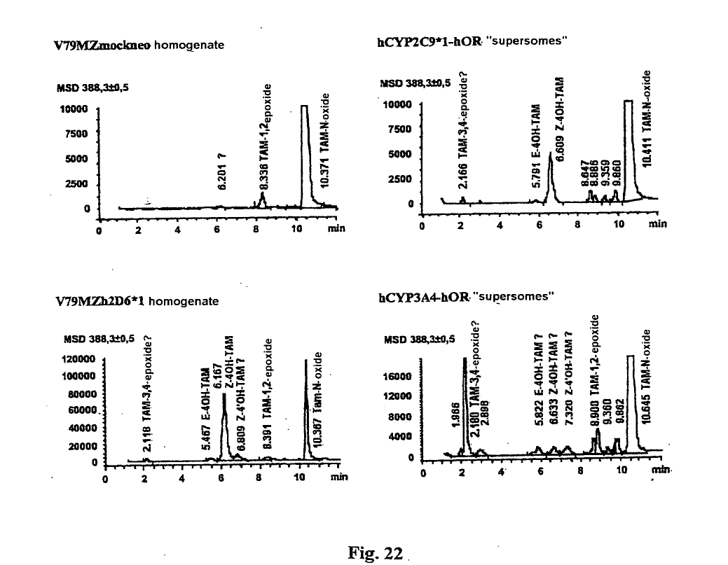

FIG. 22 shows HPLC/MSD profiles after incubation with hCYP2C9*1-hOR and hCYP3A4-hOR “supersomes” as well as with homogenate of cell line V79MZh2D6*1 in comparison to the negative control V79MZmockneo.

FIG. 23 schematically shows a detail of the metabolism of Z-tamoxifen in human liver. The 4-hydroxylation reaction of tamoxifen is highlighted horizontally while the different metabolites tested having modifications at the nitrogen are highlighted vertically. Adapted according to (IARC, 1996).

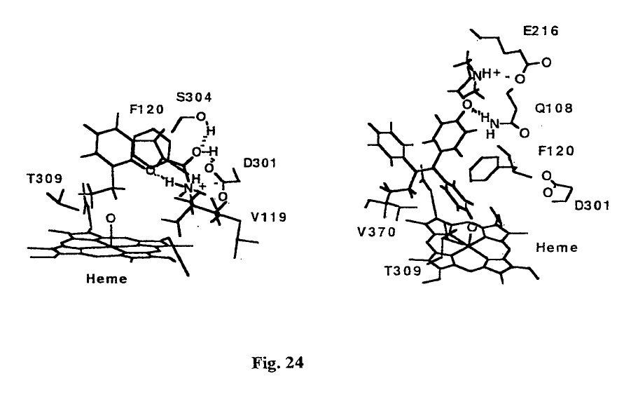

FIG. 24 shows a 3D homology model of the binding of bufuralol and tamoxifen to the active site of hCYP2D6 according to-Lewis (1998). Shown are the two substrates, the oxygen atom transferred to the substrate. the water bridges stabilizing the substrate in the active site, and the characteristic ionic interaction between the protonated nitrogen of the substrate and Asp₃₀₁ or Glu₂₁₆, respectively, of the enzyme.

### EXAMPLES

The following Examples illustrate the present invention but should not be construed as limiting.

### Example 1

### Vector Construction for the Stable Expression of hCYP2D6 cDNAs in V79 Chinese Hamster Cells

According to the present invention, three strategies were used for the stable expression of hCYP2D6 cDNA in V79 Chinese hamster cells:
Cotransfection of the pSV450h2D6 expression vector and the neomycin resistance Plasmid pSV2neo (Clontech Laboratories, Inc., Palo Alto, Calif.) and selection with geneticin 418 (geneticin 418-sulfate; Calbiochem-Novabiochem Corp., La Jolla, Calif.) according to Doehmer et al. (1988).
Transfection with plasmid pcDNA3.1Hygro(+)h2D6 and selection with hygromycin B (Boehringer Mannheim GmbH, Mannheim). The cDNA and the hygromycin resistance were combined on one vector to reduce the amount of DNA necessary for transfection and to examine the effect on chromosomal integrity following transfection. Furthermore, the direct coupling of cDNA and resistance gene was performed to increase the fraction of positive clones obtained after transfection.
Cotransfektion of expression vector pSV450h2D6 and plasmid pRc/RSVhCYPOR (Schneider et al., 1996) and selection with geneticin 418 according to Schneider et al. (1996). Vector pRc/RSVhCYPOR carries a neomycin resistance gene and a hCYPOR cDNA. The coexpression of hCYP2D6 with hCYPOR was performed to demonstrate whether the endogenous amount of CYPOR in V79MZ cells is sufficient for maximal hCYP2D6 activity.

The cDNAs for the alleles hCYP2D6*1 in pBluescript SK(+) (Stratagene, Heidelberg), hCYP2D6*2 in pVL1393 (Invitrogen Corp., Carlsbad, Calif.), hCYP2D6*9 in M13mp19 (Stratagene, Heidelberg) and hCYP2D6*10A in M13mp19 were provided courtesy of Dr. U. M. Zanger (Dr. Margarete Fischer-Bosch-Institut, Stuttgart). The alleles are shown in FIG. 1 and may be obtained also by means of standard methods.

A. Construction of pSV450 Polylinker Vectors

For a stable transfection of V79 Chinese hamster cells the expression vector pSV450 (Doehmer et al., 1988) was used. To simplify the cloning of cDNAs into this vector, the pSV450 vectors were constructed with different polylinkers. In a first step, the polylinkers for vectors pSV450HB and pSV450HK were generated by means of PCR (Saiki et al., 1988) according to PCR#1 and for vector pSV450HS by means of touch down PCR according to PCR#2.

PCR#1

Sample: primer: 1.4 μl 16298 (25 mM), 1.4 μl 16299 (25 mM); template: 1 μl 16300 (40 μM); 1 μl dNTPs (20 mM); 0.4 μl Taq DNA polymerase (5 U/μl, QIAGEN, Hilden); 2.5 μl PCR buffer (10×); ad 25 μl with water

PCR device: Gene Amp PCR System 2400 (Perkin Elmer, Norwalk Conn., USA) temperature program: 2 min 48° C., 1 min 72° C. (1×); 1 min 94° C., 1 min 55° C., 1 min 72° C. (30×); 10 min 72° C. (1×)

amplificate: 84 bp

PCR#2

Sample: primer: 1.4 μl 19176 (25 mM), 1.4 μl 19177 (25 mM); template: 1 μl 19183 (40 μM); 1 μl dNTPs (20 mM); 0.4 μl Taq DNA polymerase (5 U/μl, QIAGEN, Hilden); 2.5 μl PCR buffer (10×); 5 μl solution Q (5×); ad 25 μl with water

PCR device: Gene Amp PCR System 2400 (Perkin Elmer, Norwalk Conn., USA) temperature program: 1 min 94° C. (1×); 1 min 94° C., 1 min 62° C.→52° C., 2 min 72° C. (10×, annealing temperature decreases in steps of 1° C.); 1 min 94° C., 1 min 52° C., 2 min 72° C. (35×); 10 min 72° C. (1×)

amplificate: 114 bp

Sequences of Primers and Templates:

| | |
|---|---|
| 16298: | 5′-TAGACAAGCTTGGATCCATG-3′ |
| 16299: | 5′-GCTATAAGCTTAGATCTCGG-3′ |
| 16300: | 5′-TAGACAAGCTTGGATCCATGGTACCGAGCTCGAGTCGACTGCAGTTAACTC |
| | TAGATCGATGCGGCCGAGATCTAAGCTTATAGC-3′ |
| 19176: | 5′-GCATTAAGCTTAAGTCGACC-3′ |
| 19177: | 5′-CCGTATGATCACTAGTAGATC-3′ |
| 19183: | 5′-GCATTAAGCTTAAGTCGACCGGTACCGTACGCTAGCGAATTCCGGATATCG |
| | ATGGCGCGCCGCGGCCGCTCGAGCTCTAGACGCGTGGATCCAGATCTACTAGTG |
| | ATCATACGG-3′ |

From the original vector pSV450r2B1 (FIG. 2; Doehmer et al., 1988) the rCYP2B1 cDNA was cut out by Hind III and Bgl II and the respective polylinker (PCR#1 digested with Hind III and Bgl II; PCR#l digested with Hind III and BamH I; PCR#2 digested with Hind III and Bcl I) was inserted by ligation. The novel vectors were named pSV450HB, pSV450HK and pSV450HS. The polylinkers of vectors pSV450HB, pSV450HK and pSV450HS have the structures shown below. All restriction sites mentioned are unique sites in the vectors.

pSV450HB: Hind III-Kpn I/Asp 718-Sac I/Ecl 136-Xho I/Ava I-Sal I-Hpa I-Xba I-Cla I-Xma III-Bgl II

pSV450HK: Hind III-Bgl II-Xma III-Cla I-Xba I-Hpa I-Sal I-Xho I/Ava I-Sac I/Ecl 136-Kpn I/Asp 718

pSV450HS: Hind III-Afl II-Sal I-Age I-Kpn I/Asp 718-BspM II-EcoR V-Cla I-BssH I-Asc I-Sac I/-Xma III-Not I-Xho I/Ava I-Sac I/Ecl 136-Xba I-Mlu I-BsaB I-Bgl II-Spe I

Thus, in combination with PCR which enables the addition of compatible ends to each cDNA the construction of novel pSV450 expression vectors can be performed in the future without time consuming intermediate cloning steps. To ensure an optimal translation of the heterologous mRNA after transfection into V79MZ cells, the integrity of the Kozak sequence should be taken into account during cloning and this sequence should be optimized, respectively (Kozak, 1987; Kozak, 1990).

B. Construction of the Vectors pSV450h2D6*1, *2, *9, *10 and *17

pSV450h2D6*1, *9 and *10

The cDNAs hCYP2D6*1 in pBluescript SK(+), hCYP2D6*9 in M13mp19 and hCYP2D6*10A in M13mp19 were excised from their original vectors by means of BamH I and EcoR I and subcloned into vector pIC19H (ATCC, Manassas, Va.) which previously was digested also with BamH I and EcoR I. The novel vectors were named pICh2D6*1, *9 and *10. Restriction of these vectors with Hind III and Bgl II generated cDNAs with compatible ends for ligation into expression vector pSV450. The novel expression vectors were called pSV450h2D6*1, *9 and *10.

According to the present invention, to confirm successful ligation of the cDNA into vector pSV450 the clones obtained after transformation of E. coli with pSV450 cDNA vectors were picked with a toothpick, resuspended in 10 μl of sterile water and subjected to the following PCR:

PCR#4: Control PCR for Successful Ligation of a cDNA into Vectors pSV450, pSV450HB, pSV450HK and pSV450HS

Sample: primer: 1.4 μl 20261 (25 mM), 1.4 μl 20262 (25 mM); template: 2 μl E. coli-suspension; 1 μl dNTPs (20 mM); 0.2 μl Taq DNA polymerase (5 U/μl, QIAGEN, Hilden); 2.5 μl PCR buffer (10×); ad 25 μl with water; 1 drop of silicone oil

PCR device: Gene Amp PCR System 2400 (Perkin Elmer, Norwalk Conn., USA)

temperature program: 3 min 94° C. (1×); 0.5 min 94° C., 1 min 56° C., 2 min 72° C. (30×); 10 min 72° C. (1×)

Amplificate: If vector pSV450 contains an insert between the restriction sites for Hind III and Bgl II, the length of the amplificate will be 72 bp+insert bp. If no insert is present, a fragment of 72 bp will be amplified. For vectors pSV450HB, pSV450HK and pSV450HS which contain the polylinker the “72 bp fragment” is extended correspondingly.

Primer Sequences:

| | |
|---|---|
| 20261: | 5′-TATTCCAGAAGTAGTGAGG-3′ |
| 20262: | 5′-ATCACCGAGCTGAGAAGC-3′ |

pSV450h2D6*2

hCYP2D6*2 cDNA was excised from vector pVL1393 (Invitrogen Corp., Carlsbad, Calif.) with Hind III and Kpn I and subcloned into vector pICh2D6*10 which previously was also digested with Hind III and Kpn I. From the novel vector pICh2D6*2 the cDNA was excised again with Hind III and Bgl II and cloned into expression vector pSV450 (Doehrmer et al., 1988). This vector was named pSV450h2D6*2.

pSV450h2D6*17

Allele hCYP2D6*17 differs from allele hCYP2D6*2 only in the mutation C₁₁₁₁T. This mutation is located immediately upstream of an Xho II restriction site (FIG. 1). Therefore, the cDNA of hCYP2D6*17 could be obtained from the hCYP2D6*2-cDNA by site. directed point mutagenesis: a cDNA fragment with a length of 362 bp carrying the mutation C₁₁₁₁T was synthesized according to PCR#3 and digested with Hind III and Sho II.

PCR#3

Sample: primer: 1.4 μl 16094 (25 mM), 1.4 μl 16095 (25 mM); template: 1 μl pSV450h2D6*1 (15.5 ng/μl); 1 μl dNTPs (20 mM); 0.125 μl Red Hot DNA Polymerase (5 U/μl, Advanced Biotechnologies Ltd., Surrey, England); 2.5 μl reaction buffer IV (10×); 1.5 μl magnesium chloride (25 mM); ad 25 μl with water; 1 drop of silicone oil PCR device: Genius (Techne Ltd., Duxford Cambridge, England)

temperature program: 1 min 94° C. (1×); 1 min 94° C., 2 min 55° C., 3 min 72° C. (30×); 10 min 72° C. (1×)

amplificate: 362 bp

Primer Sequences:

| | |
|---|---|
| 16094: | 5′-AGACGTGAAGCTTGCCGCCACCATGGGGCTA-3′ |
| 16095: | 5′-CAGGACGTAGAATGGATCTGGATGATGGGCAC-3′ |

The second cDNA fragment was obtained from plasmid pICh2D6*2 using Xho II and Bgl II. In a three-component ligation, both cDNA fragments were cloned together with Hind III and Bgl II restricted expression vector pSV450. The novel vector was called pSV450h2D6*17.

C. Construction of Vectors pcDNA3.1Hygro(+)h2D6*1, *2, *9,*10 and *17

The cDNAs hCYP2D6*1, *2, *9, *10 and * 17 were excised from the respective pSV450h2D6 plasmid using Hind III and Bgl II and cloned into the expression vector pcDNA3.1Hygro(+) (Invitrogen Corp., Carlsbad, Calif.) digested with Hind III and BamH I. The novel vectors were called pcDNA3.1Hygro(+)h2D6*1, *2, *9, *10 and *17.

To confirm the successful ligation of the cDNA into vector pcDNA3.1Hygro(+) the clones obtained following transformation of E. coli with pcDNA3.1Hygro(+) cDNA vectors were picked with a toothpick, resuspended in 10 μl of sterile water and subjected to the following PCR:

PCR#5: Control PCR for Successful Ligation of a cDNA into Vector pcDNA 3.1Hygro(+)

Sample: primer: 1.4 μl 18383 (25 mM), 1.4 μl 18384 (25 mM); template: 2 μl E. coli suspension; 1 μl dNTPs (20 mM); 0.2 μl Taq DNA polymerase (5 U/μl, QIAGEN, Hilden); 2.5 μl PCR buffer (10×); ad 25 μl with water

PCR device: Gene Amp PCR System 2400 (Perkin Elmer, Norwalk Conn., USA)

temperature program: 3 min 94° C. (1×); 0.5 min 94° C., 1 min 56° C., 2 min 72° C. (30×); 10 min 72° C. (1×)

amplificate: If the vector pcDNA3.1Hygro(+) contains an insert the length of the amplificate will be 200 bp+insert bp. If no insert is present a fragment of 203 bp will be amplified.

Primer Sequences:

| | |
|---|---|
| 18383: | 5′-CACTGCTTACTGGCTTATCG-3′ |
| 18384: | 5′-ACTAGAAGGCACAGTCGAGG-3′ |

### Example 2

### Transfection

According to the present invention, parental V79MZ cells were transfected with recombinant expression vectors by potassium phosphate coprecipitation (Graham and Van der Eb, 1973; Parker and Stark, 1979).

According to the invention, V79MZ cells were cultured at 37° C., 7% CO₂ and a humidity of 90% in tissue culture flasks (94/16 mm or 145/20 mm tissue culture dishes and 50 ml or 250 ml tissue culture flasks obtained from Greiner GmbH, (Frickenhausen); 24 well and 96 well tissue culture microtiter plates obtained from Nunc Inc. (Naperville, Ill.)) having a special coating in DMEM culture medium with increased glucose (4.5 g/l). In addition, the DMEM culture medium was supplemented with 1 mM sodium pyruvate, 4 mM L-glutamine, 10% FCS, 100 U/ml penicillin, and 100 μg/ml streptomycin (“complete medium”). Following transfection, geneticin 418- or hygromycin B-resistant V79MZ cell clones were cultured in complete medium with 0.5 mg geneticin 418/ml, or complete medium with 0.4 mg hygromycin B/ml, respectively.

For transfections, the amounts of DNA used were added with 500 μl HEPES (Gibco BRL, Eggenstein) buffered saline (137 mM sodium chloride, 6 mM dextrose, 5 mM potassium chloride, 0.7 mM sodium hydrogen phosphate, 20 mM HEPES, pH 7.0). By addition of 26 μl of 2.5 M calcium chloride solution and incubation for 30 min at room temperature the DNA was coprecipitated on calcium phosphate. The precipitate was added dropwise to a culture of 1.5-2×10⁶ parental V79MZ cells in a 145/20 mm tissue culture dish. After careful mixing and incubation for 4 hours at 37° C., the cells were incubated for 2 min with 15% (v/v) glycerol in complete medium to increase the effectiveness of DNA uptake. Subsequently, the glycerol was removed by aspiration of the medium and washing twice with 7 ml each of complete medium. Since the cells have to pass through the cell cycle for stable integration of foreign DNA into their genome they were first incubated for about 36 h in complete medium without G418 or hygromycin B. Afterwards, the cells were carefully trypsinized, suspended in 1 mg of 1 mg/ml G418 complete medium or 0.4 mg/ml hygromycin B complete medium, respectively, and distributed on three 96 well tissue culture microtiter plates. Only cells which had taken up a resistance gene against G418 or hygromycin, respectively, during transfection with the vectors were able to survive (Mulligan and Berg, 1981). After 10-14 days the resistant cell clones could be observed. Individual clones were trypsinized directly in the well and half of the cells were removed for in situ immunofluorescence.

The clonality and stability was confirmed by repeated subcloning and passaging (the majority of the trypsinized cells was discarded or transferred to new culture flasks) of the novel cell lines as well as by repeated in situ immunofluorescence and determination of the enzymatic activity.

The different transfection samples T1-T14 performed according to the present invention as well as the cell lines are summarized in Table 2.

Construction of Cell Lines V79MZh2D6*1, *2, *9, *10 and *17:

30 μg of Sca I linearized pSV450h2D6*1, *2, *9, *10 or *17 DNA and 1 μg of EcoR I linearized pSV2neo DNA were transfected. In transfections of resistance vector pSV2neo, the expression vector pSV450h2D6 was used in a 30fold excess to increase the probability of obtaining a geneticin 418-resistant and at the same time hCYP2D6-expressing clone (T1-T5). 30-50 resistant clones were obtained per sample. On average, one of 50 clones showed a homogenous expression of hCYP2D6 while about half of the clones were heterogenous in the in situ immunofluorescence and the rest did not express hCYP2D6 at all. Cell lines V79MZh2D6*1, V79MZh2D6*2, V79MZh2D6*9, V79MZh2D6*10 and V79MZh2D6*17 were deposited on Feb. 15, 2000, at the DSMZ-Deutsche Sammlung von Mikroorganismen und Zellkulturen GmbH under the accession numbers DSM ACC2446, DSM ACC2447, DSM ACC2448, DSM ACC2449 and DSM ACC2450.

Construction of Cell Lines V79MZh2D6*1-H, *2-H, *9-H, *10-H and *17-H:

3 μg of Ssp I linearized pcDNA3.1Hygro(+)h2D6*1, *2, *9, *10 or *17 DNA was transfected. The combination of the cDNA and the resistance gene on a single vector enabled a transfection with only 3 μg of DNA (T6-T10). 10-30 resistant clones were obtained per sample. On average, one of 10 clones showed a homogenous expression of hCYP2D6 while about two thirds of the clones were heterogenous in the in situ immunofluorescence, the rest did not express hCYP2D6 at all.

Thus, the amount of V79MZ clones showing a homogenous expression of hCYP2D6 could be enhanced by 5fold as compared to the cotransfection of pSV450h2D6 and pSV2neo. This is important for the present method because the identification of clones showing a homogenous expression of the cDNA is the time-limiting step in the construction of novel cell lines.

Construction of Cell Line V79MZh2D6*1-hOR:

30 μg of Sca I linearized pSV450h2D6*1 DNA and 1 μg of Sca I linearized pRc/RSV-hCYPOR DNA were transfected. In comparison to cell line V79MZh2D6*1 (T1) the coexpression of hCYP2D6*1 and hCYPOR (T11) should show whether the CYPOR content of V79MZ cells is sufficient for maximal hCYP2D6 activity. As in the case of transfection samples T1-T5, the expression vector pSV450h2D6*1 was employed in a 30fold excess over resistance vector pRc/RSV-hCYPOR. With this approach, 34 resistant clones were obtained one of which showed a homogenous expression of both hCYP2D6*1 and hCYPOR.

Construction of the Mock-transfected Cell Lines V79MZmockneo:

The following transfections were carried out:
a) 30 μg of EcoR I linearized pSV2neo DNA
b) 1 μg of EcoR I linearized pSV2neo DNA
c) 1 μg of EcoR I linearized pSV2neo DNA and 30 μg of Sca I linearized pSV450HB DNA

Depending on the amount of DNA transfected, the chromosomal integrity of the recipient cell may be disturbed and chromosomal aberrations may be caused by recombination (Bradwell, 1989). Therefore, in the context of the construction of mock-transfected V79MZ cells the transfection was performed with various amounts of DNA. The karyotype of the resulting cell clones was characterized.

If the transfection was carried out with only 1 μg pSV2neo (T13) as well as with 1 μg pSV2neo and 30 μg pSV450HB (T14) the number of clones counted was the same as in samples (T1-T5). Thus, the addition of pSV450HB as a “carrier DNA” (Graham and Van der Eb, 1973; Strain and Wylie, 1984) had no effect on the number of geneticin 418-resistant clones. About 120 clones were obtained with 30 μg pSV2neo DNA, i.e. a 30times higher amount of DNA yielded only 3-4times more clones.

Among 3×6 randomly selected clones, one clone with altered morphology was identified in the microscope. A similar ratio was also observed for the other transfections. Morphologically altered clones (FIG. 8) were excluded from further characterizations and were discarded.

The cell line V79MZmockneo130 was used as a negative control according to the present invention in addition to the parental V79 cells. In the following it will be referred to as “V79MZmockneo”.

TABLE 2
Summary of transfection samples and novel cell lines.

| | | Heterologous | | Expression | Resistance |
|---|---|---|---|---|---|
| | Cell line | expression | Res. | vector (μg) | vector (μg) |
| T1 | V79MZh2D6*1 | hCYP2D6*1 | G418 | pSV450h2D6*1 (30) | pSV2neo (1) |
| T2 | V79MZh2D6*2 | hCYP2D6*2 | G418 | pSV450h2D6*2 (30) | pSV2neo (1) |
| T3 | V79MZh2D6*9 | hCYP2D6*9 | G418 | pSV450h2D6*9 (30) | pSV2neo (1) |
| T4 | V79MZh2D6*10 | hCYP2D6*10 | G418 | pSV450h2D6*10 (30) | pSV2neo (1) |
| T5 | V79MZh2D6*17 | hCYP2D6*17 | G418 | pSV450h2D6*17 (30) | pSV2neo (1) |
| T6 | V79MZh2D6*1-H | hCYP2D6*1 | HyB | pcDNA3.1Hygro(+)h2D6*1 (3) | |
| T7 | V79MZh2D6*2-H | hCYP2D6*2 | HyB | pcDNA3.1Hygro(+)h2D6*2 (3) | |
| T8 | V79MZh2D6*9-H | hCYP2D6*9 | HyB | pcDNA3.1Hygro(+)h2D6*9 (3) | |
| T9 | V79MZh2D6*10-H | hCYP2D6*10 | HyB | pcDNA3.1Hygro(+)h2D6*10 (3) | |
| T10 | V79MZh2D6*17-H | hCYP2D6*17 | HyB | pcDNA3.1Hygro(+)h2D6*17 (3) | |
| T11 | V79MZh2D6*1-hOR | hCYP2D6*1 | G418 | pSV450h2D6*1 (30) | pRc/RSV |
| | | hCYPOR | | | -hCYPOR (1) |
| T12 | V79MZmockneo30 | | G418 | | pSV2neo (30) |
| T13 | V79MZmockneo1 | | G418 | | pSV2neo (1) |
| T14 | V79MZmockneo130 | | G418 | pSV450HB (30) | pSV2neo (1) |

Abbreviations:
Res, resistance; G418, geneticin 418; HyB, hygromycin B

### Example 3

### Characterization of the Novel Cell Lines

In Situ Immunofluorescence

For the detection of the heterologous expression of hCYP2D6 and/or hCYPOR the clones obtained after transfection were characterized by in situ immunofluorescence (FIG. 9). Only homogenous clones in which all cells were stained homogenously and intensely were subjected to further cultivation. Other criteria for the selection were a structured stain showing the subcellular localization of hCYP2D6 and hCYPOR in the endoplasmic reticulum (FIG. 9c) as well as a characteristic darker nuclear region.

For the in situ immunofluorescence, 10⁴ cells of the clones to be tested were seeded on microchamber slides (Nunc Inc., Naperville, Ill.) and cultured for 24 h. Afterwards, the chambers were removed and the cells adhered on the slides were washed with PBS (Bio Whittaker, Verviers, Belgium) and incubated with icecold methanol/acetone (1:1) for 7 min for fixation, and then dried in air. The cells fixed in this manner were covered with 150 μl of primary antibody solution (polyclonal anti-hCYP2D6 antiserum 637.2 from rabbits, diluted 1:200 in complete medium, provided courteously by Dr. U. M. Zanger, Dr. Margarete Fischer-Bosch-Institut, Stuttgart), covered with polyethylene foil and incubated for 90 min at room temperature. Subsequently, they were washed 3times each for 10 min with PBS, 150 μl of secondary antibody solution (FITC-coupled anti-rabbit IgG antibody from goat, 1.5 mg/ml, diluted 1:125 in complete medium, Dianova, Hamburg) was applied, covered with polyethylene foil and incubated for 1 h at room temperature in the dark. After washing three times with PBS for 10 min each 100 μl of “antifading” reagent (100 mg p-phenylene diamrmoniumdichloride in 10 ml PBS and 80 ml glycerol) was applied and a cover slip was placed on top without capture of air bubbles. The samples were evaluated using a fluorescence microscope (Axioplan, Carl Zeiss, Oberkochen) with a set of standard filters at an excitation range of 450-490 nm. To demonstrate the subcellular localization of cytochrome P450, confocal sections were observed using a Laser Scanning Microscope LSM 4.10 (Carl Zeiss, Oberkochen) with water immersion. The fluorescence was excited at 488 nm and the emission detected at 515-565 nm. Accordingly, the cytochrome expressed accordingly showed a green stain.

For detecting the coexpression of hCYPP2D6 and hCYPOR by double staining additionally anti-hCYPOR antibody from goat was added to the primary antibody solution (final dilution 1:500 in complete medium). To avoid cross reactions a mixture of TRITC-coupled anti-rabbit IgG antibody from mouse (1.5 mg/ml, 1:125 dilution in complete medium, Pierce, Rockford, Ill.) and FITC coupled anti-goat IgG antibody from mouse (1.5 mg/ml, 1:125 dilution in complete medium, Sigma, Deisenhofen) was used as secondary antibody solution. Subsequently in a double stain the oxidoreductase showed a green and the cytochrome P450 showed a red stain.

Selection of Representative Clones

The in situ immunofluorescence identified 1-6 clones of each cell line which were homogenous with respect to their cDNA expression. For a detailed characterization and later use, one representative clone for each transfection sample was selected from these clones. Positive criteria for the selection were an unchanged morphology and a doubling time similar compared to that of the parental cell line V79MZ. Eventually, clones were preferred which showed an intermediate hCYP2D6 activity. For this purpose, the specific hydroxylation of (±)-bufuralol was measured for all clones.

Clones with an intermediate hCYP2D6 activity were selected to minimize distortions in the allele specific activities due to the site of cDNA integration or a (rare) multiple integration since in contrast to a homologous integration the site of cDNA integration in the V79MZ genome is to a certain extend random (Schulz et al., 1987). So-called “chromatin effects” and adjacent sequence regions may affect the transcription of the integrated cDNA (Butner and Lo, 1986; Jaenisch and Jahner, 1984; Wahl et al., 1984). Therefore, the amount of hCYP2D6 expressed in a heterologous manner and thus the enzyme activity per mg of cellular protein is dependent on the site of integration of the cDNA.

Accordingly, the bufuralol hydroxylase activities of different clones of one cell line differed up to threefold. In the case of transfection samples T2 (cell line V79MZh2D6*2) and T11 (cell line V79MZh2D6*1-hOR) a selection with respect to the mean activity was impossible since only one clone in each sample fulfilled all other criteria. The clones selected are identical to the novel cell lines, and only these clones were subjected to a detailed characterization.

Detection of the cDNAs Integrated into the Genome

To confirm the genomic integration of the cDNAs transfected, the genomic DNA was isolated from.cell lines V79MZh2D6*1, *2, *9, *10 and *17 (deposited on Feb. 15, 2000, at the DSMZ-Deutsche Sammlung von Mikroorganismen und Zellkulturen GmbH under the accession numbers DSM ACC2446, DSM ACC2447, DSM ACC2448, DSM ACC2449 and DSM ACC2450), the hCYP2D6 expression cassette was amplified according to PCR#6 by means of touch down PCR in the form of an amplificate of 2198 and 2299 bp, respectively, and detected by means of gel electrophoresis.

For the isolation of genomic DNA from V79MZ cells the V79MZh2D6 cells were grown in 94/16 mm tissue culture dishes up to a confluence of almost 100%, the medium was removed and washed twice with 5 ml PBS. Afterwards, the genomic DNA was isolated using the QIAamp Blood Midi kit (QIAGEN, Hilden) according to the protocol of the manufacturer.

PCR#6

Sample: primers: 1.4 μl 16014 (25 mM), 1.4 μl 15889 (25 mM); template: 1 μl of genomic DNA (approx. 0.2 μg/μl); 1 μl dNTPs (20 mM); 0.5 μl Taq DNA polymerase (5 U/μl, QIAGEN, Hilden); 2.5 μl PCR buffer (10×); 5 μl solution Q (5×); ad 25 μl with water

PCR device: Gene Amp PCR System 2400 (Perkin Elmer, Norwalk Conn., USA)

temperature program: 1 min 94° C. (1×); 1 min 94° C., 1 min 62° C.→52° C., 3 min 72° C. (10×, annealing temperature decreases in steps of 1° C.); 1 min 94° C., 1 min 52° C., 3 min 72° C. (35×); 10 min 72° C. (1×)

Amplificate: Depending on the hCYP2D6 cDNA inserted an amplificate having a length of 2198 or 2299 bp, respectively, is obtained. Without insert (mock transfection) the amplificate has a length of about 700 bp depending on the pSV450 vector integrated.

Primer Sequences:

| | |
|---|---|
| 16014: | 5′-CAGAGGTTTTCACCGTCATC-3′ |
| 15889: | 5′-GGAATGTCCTCTCAAGTAGA-3′ |

To confirm the identity of the five alleles, the amplificates were sequenced using the primers 16094 (5′-AGACGTGAAGCTTGCCGCCACCATGGGGCTA-3′), 15887 (5′-AGCTGGATGAGCTGCTAA-3′) and 15888 (5′-ATCACCAACCTGTCATCGG-3′). This also served to simultaneously confirm the integrity of the transfected DNA which is extremely susceptible to mutations, particularly deletions, until it is integrated into the genome (Bradwell, 1989; Calos et al., 1983).

Detection of the hCYP2D6 mRNA

The transcription of the integrated hCYP2D6 cDNAs was verified on the level of mRNA by means of RT-PCR#1.

For the isolation of total RNA from V79MZ cells, the V79MZh2D6 cells were grown in 94/16 mm tissue culture dishes until a confluence of about 90% was observed followed by removal of the medium and washing twice with 5 ml PBS. Subsequently, the total RNA was isolated using 1.5 ml of peqGOLD TriFast™ solution (peqLab Biotechnologie GmbH, Erlangen) according to the protocol of the manufacturer.

RT-PCR#1

The PCR was performed using the Access RT-PCR system (Promega Corp., Madison, Wis.):

Sample: primers: 2 μl 16093 (25 mM), 2 μl 17606 (25 mM); template: 0.5 μl of isolated total RNA (1-2 μg/μl); 1 μl dNTPs (10 mM); AMV reverse transcriptase (5 U/μl); 1 μl Tfl DNA polymerase (5 U/μl); 10 μl AMV/Tfl reaction buffer (5×); 2 μl magnesium sulfate (25 mM); ad 50 μl with DEPC (Sigma, Deisenhofen) treated water

PCR device: Uno Thermoblock (Biometra biomedizinische Analytik GmbH, Göttingen)

temperature program: 50 min 48° C. (1×); 2 min 94° C. (1×); 1 min 94° C., 1 min 60° C., 2 min 70° C. (40×); 10 min 70° C. (1×)

Amplificate: A 410 bp fragment of reverse transcribed hCYP2D6 mRNA is amplified.

Primer Sequences:

| | |
|---|---|
| 16093: | 5′-CATACTGCTTCGACCAGTTGCG-3′ |
| 17606: | 5′-GCAGGTGAGGGAGGCGATCAC-3′ |

Western Blot

After the homogenous expression von hCYP2D6 in the V79MZh2D6 cell lines according to the invention was confirmed by means of in situ immunofluorescence, the molecular weight of heterologously expressed hCYP2D6 was confirmed by Western blotting and a qualitative indication as to the relative amounts of hCYP2D6 was obtained (FIG. 11).

For this purpose, the proteins of a cell homogenate (see Example 4) separated on SDS PAGE (Laemmli, 1970) were blotted from the polyacrylamide gel onto an Immobilon P membrane (Millipore, Dreieich) (Burnette, 1981) for 30 min using a “semi dry” method at 225 mA in a “Semi Dry Elektroblotter” device (Sartorius, Göttingen). For the transfer, a pile was prepared starting from the graphite anode which consisted of two pieces of Whatman 3 MM filter papers soaked in buffer C (0.03 M Tris, 20% methanol, 0.04 M 6-aminohexanoic acid, ad pH 10 with aqueous sodium hydroxide), the gel which had been previously immersed for 15 min in buffer C, the membrane which was wetted previously with methanol and then soaked for 10 min in buffer B (0.03 M Tris, 20% methanol, ad pH 10 with aqueous sodium hydroxide), two pieces of Whatman 3 MM paper soaked in buffer B and two pieces of Whatman 3 MM paper soaked in buffer A (0.3 M Tris, 20% methanol, ad pH 10 with aqueous sodium hydroxide) without inclusion of air bubbles and then was fixed by the graphite cathode.

For the immunodetection of hCYP2D6, the blotted membrane was blocked over night in PBS, 7% skim milk powder (“Glücksklee” trademark, Nestlé Germany AG, Frankfurt) at 4° C., washed briefly in PBS and then shaken carefully for 1 h in a solution of the primary antibody (polyclonal anti-hCYP2D6 antiserum 637.2 from rabbits, diluted 1:100 in PBS), and afterwards washed three times for 15 min in PBS, 0.5% Tween 20, incubated for 30 min in the secondary antibody solution (POD-coupled anti-rabbit IgG from goat, 0.2 U/ml, diluted 1:10000 in PBS, Boehringer Mannheim, Mannheim) and washed again three times for 15 min in PBS, 0.5% Tween 20. Afterwards, hCYP2D6 was detected via the bound secondary antibody using the ECL kit (Enhanced Chemiluminescence, Amersham, Little Chalfont, England). This assay detects the light emission during the peroxidase catalyzed oxidation of luminol in the presence of hydrogen peroxide using Hyperfilm ECL (Amersham, Little Chalfont, England).

With 56.0±0.6 kDa and 80.3±2.8 kDa, the experimental molecular weights of hCYP2D6 and hCYPOR closely corresponded to the calculated values of 55.8 kDa and 76.7 kDa, respectively. Remarkable is the relatively low amount of hCYP2D6 10; see FIG. 11.

### Example 4

### CO Difference Spektra

For a comparison of the different hCYP2D6 1 expressing cell lines V79MZh2D6*1, -hOR, -H and -S as well as the various allelic variants, the amounts of cytochrome P450 was determined using CO difference spectra.

For the preparation of a cell homogenate, the V79MZ cells were cultured in at least three 250 ml tissue culture flasks up to a confluence of 80-100%. The trypsin-treated cells were combined, distributed uniformly on three 145/20 mm tissue culture dishes each per 250 ml tissue culture flask and incubated in 20 ml of complete medium without G418 or hygromycin B up to a confluence of 90%. The medium was discarded, followed by rinsing twice with 5 ml icecold buffer (100 mM potassium phosphate, pH 7.4). The cells were loosened from the dish by means of a rubber scraper into 4 ml of icecold buffer, combined and pelleted by centrifugation for 10 min at 1500×g and 4° C. The supernatant was completely removed, the pellet was carefully resuspended in 1 ml buffer per nine 145/20-mm tissue culture dishes, and aliquots were taken as follows: 1 ml of the cell suspension was removed for CO difference spectra (1 ml aliquot), the remainder was diluted 1:1 with buffer, resuspended, and divided into aliquots of 100-300 μl for the determination of the protein content, enzymatic activity, measurements of enzyme kinetics, and for Western blotting (diluted aliquots). All aliquots were shock frozen in liquid nitrogen to disrupt the cells and afterwards stored at −80° C. until use.

A 1 ml aliquot of cell homogenate was thawed on ice and after addition of 20 μl 100 mM PMSF (Boehringer Mannheim GmbH, Mannheim) in isopropanol was carefully resuspended in 1 ml of solubilization buffer (100 mM sodium hydrogenphosphate, pH 7.4, 10% (v/v) glycerol, 0.5% (w/v) emulgen 913 (Kao-Atlas, Tokyo, Japan) using a pipette. For the measurement of the hCYP2D6 spectra there were also added 10 μl of 2 mM quinidine (hydrochloride, Sigma, Deisenhofen). Membrane-bound cytochrome was solubilized by careful stirring for 15 min on ice, and insoluble material was pelleted by centrifugation for 10 min at 17,000×g at 4° C. to reduce the turbidity (Evert et al., 1997; Tyndale et al., 1991a).

The supernatant was introduced into a 2 ml glas-on-glas homogenizator, reduced by adding several crystals of sodium dithionite (Sigma, Deisenhofen) followed by 15 strokes, and split up between two quartz cuvettes. After recording of the reduced spectrum between 400 and 500 nm by means of an Aminco DW-2000 UV/VIS spectrophotometer (SLM Instruments Inc., Urbana, Ill.) the solution in the test cuvette was saturated with about 60 bubbles of carbon monoxide and the CO/reduced spectrum was recorded immediately (Eastabrook et al., 1972). From the two spectra the CO/reduced versus the reduced spectrum (CO difference spectrum) was evaluated, and using the extinction coefficients of 91 mM⁻¹ cm⁻¹ and 110 mM⁻¹ cm⁻¹ (Omura and Sato, 1964b) the concentrations of cytochrome P450 and cytochrome P420 were calculated.

In the absence of solubilization and centrifugation, the turbidity of the cell homogenate was to high for the measurement of CO spectra. After centrifugation without previous solubilization, all of the cytochrome P450 was found in the pellet. If the method according to the present invention using the non-ionic detergent emulgen 913 was employed, however, cytochrome P450 was almost completely solubilized (FIG. 12B). After a centrifugation to remove the turbidity was carried out cytochrome P450 could be well measured in the solubilizate. A final concentration of emulgen 913 of 0.25% was found to be optimal. In addition, also a repeated treatment of the resuspended pellet with emulgen 913 did not solubilize any more cytochrome P450. The solubilization was independent of the incorporation of heme which becomes clear particularly from the solubilization of hCYP2D6 9 after culturing in the presence and absence of quinidine (FIGS. 12B and 14A).

If the expression was performed in baculovirus-infected insect cells, however, the solubilization with emulgen 913 yielded a maximum of about 50% of hCYP2D6 9 (Evert et al., 1997; Paine et al., 1996). The variant hCYP2D6 7 which is unable to incorporate heme because of a His₃₂₄Pro mutation and therefore is not functional remained practically unsolubilized (Evert et al., 1997). Also in these two cases, no dependence on exogenous heme was observed. For the solubilization of baculovirus-expressed bCYPc17, however, a marked dependence on heme incorporation has been reported (Barnes et al., 1994). This indicates that also other factors besides the association with heme must affect the solubilization, for example the intracellular localization of the cytochrome P450 expressed in a heterologous manner, its tendency to form aggregates, the composition of the intracellular membrane systems of the expression system or the type of binding of cytochrome P450 within the membrane.

Aerobic Measurement

A reduction of the solubilizate with sodium dithionite under aerobic conditions may weaken and destroy the prosthetic heme (Omura and Sato, 1964b). Accordingly, recording a CO spectrum at different times after gassing of the sample with carbon monoxide showed a clear decrease of the absorption at 450 nm while the peak of cytochrome P420 increased. At the same time it could be demonstrated, however, that under aerobic conditions the error was neglegible if the spectra were run only a few minutes after reduction of the sample (FIG. 13).

Effect of Quinidine on the Stability of hCYP2D6

Sometimes enzymes may be stabilized by their substrates or competitive inhibitors. For hCYP2D6, a stabilization by the competitive inhibitor quinidine has been discussed (Gillam et al., 1995). A more or less noticeable peak of cytochrome P420 was observed in recordings of the spectra of hCYP2D6 2, 9, 10 and 17 which was absent in the spectrum of hCYP2D6 1. Therefore, quinidine was added to the solubilization buffer to avoid a possible weakening caused by the solubilization. The spectra, however, were more or less unchanged and particularly a cytochrome P420 peak still appeared. This means that cytochrome P450 must have been degraded already prior to solubilization and reduction.

For the detection, the quinidine for recording the spectra was added directly to the culture medium during the cultivation of the cells. A stabilizing effect was indeed observed which varied strongly with different hCYP2D6 alleles (Table 3). The spectrum of hCYP2D6 1 (wildtype) remained unchanged. The amount of cytochrome P450 was the same. This has been expected because the enzyme has a high stability by itself. In variant hCYP2D6 2 the cytochrome P420 peak was absent. The calculated amount of cytochrome P450 exactly corresponded to the sum of the amounts of cytochrome P450 and cytochrome P420 determined previously (Table 3). The observations in the case of hCYP2D6 17 were similar while the cytochrome P420 peak, however, did not completely disappear. As expected from the results of the Western analysis, it was difficult to quantify the CO difference spectrum of hCYP2D6 10 because of the low amount of protein. No cytochrome P450 peak could be observed. If the incubation was performed in the presence of quinidine the cytochrome P420 content appeared to be slightly elevated. A dramatic change was observed with variant hCYP2D6 9. The cytochrome P420 peak completely disappeared, and the amount of cytochrome P450 was increased by 4-6fold (FIG. 14A).

In contrast, as with all other variants a comparative Western blot indicated only a slight if at all increase in the amount of hCYP2D6 9 apoprotein (FIG. 14B). Thus, quinidine is not important for the stability of the apoprotein but stabilizes the prosthetic heme in the cytochrome. The solubilization of the hCYP2D6 variants examined was essentially unaffected by quinidine or heme incorporation, respectively (FIG. 12B).

Cytochrome P450 Content

The amounts of cytochrome P450 determined are summarized in Table 3.

TABLE 3
Cytochrome P450 content of different V79MZ cell lines. The values given are either
obtained from a single measurement or are the mean values and standard deviations of at
least three independent measurements.

| | without quinidine | | | with quinidine | | |
|---|---|---|---|---|---|---|
| | (pmol/mg cellular protein) | | | (pmol/mg cellular protein) | | |
| Cell line | P450 | P420 | P450 + P420 | P450 | P420 | P450 + P420 |
| V79MZmockneo | 0 | 0 | 0 | — | — | — |
| V79MZhOR | 0 | 0 | 0 | — | — | — |
| V79MZh1A1 | 11.4 ± 1.8 | 0 | 11.4 ± 1.8 | — | — | — |
| V79MZr1A1 | 6.8 ± 1.9 | 5.6 ± 2.7 | 12.4 ± 4.7 | — | — | — |
| V79MZm1A1 | 7.6 ± 1.9 | 2.6 ± 0.8 | 10.2 ± 2.0 | — | — | — |
| V79MZf1A1 (scup) | 2.5 ± 0.8 | 3.5 ± 1.6 | 6.1 ± 2.4 | — | — | — |
| V79MZh2E1 | 5.2 ± 0.6 | 0.9 ± 1.0 | 6.1 ± 0.5 | — | — | — |
| V79MZh3A4-hOR | 8.0 ± 1.0 | 3.0 ± 0.9 | 11.0 ± 1.7 | — | — | — |
| V79MZh2D6*1-hOR | 41.9 | 0 | 41.9 | 44.0 | 0 | 44.0 |
| V79MZh2D6*1-S | 17.4 ± 1.8 | 0 | 17.4 ± 1.8 | 14.41 | 0 | 14.41 |
| V79MZh2D6*1-H | 20.3 | 0 | 20.3 | 10.83 | 0 | 10.83 |
| V79MZh2D6*1 | 24.9 ± 3.2 | 0 | 24.9 ± 3.2 | 30.9 | 0 | 30.9 |
| V79MZh2D6*2 | 10.9 ± 4.3 | 2.5 ± 2.2 | 13.4 ± 4.7 | 12.2 | 0 | 12.2 |
| V79MZh2D6*9 | 5.4 ± 2.4 | 1.0 ± 0.9 | 6.4 ± 1.9 | 35.7 ± 13.3 | 0 | 35.7 ± 13.3 |
| V79MZh2D6*10 | 0 | 2.2 ± 0.2 | 2.2 ± 0.2 | 0 | 4.44 | 4.44 |
| V79MZh2D6*17 | 4.3 ± 0.7 | 3.8 ± 0.7 | 8.2 ± 1.0 | 9.0 | 1.9 | 10.9 |

Abbreviations:
—: not examined

No cytochrome P450 could be detected in parental and mock transfected V79MZ cells. The relative amounts of total cytochrome P450+cytochrome P420 corresponded well to the relative band intensities in the Western blots (FIGS. 12B and 14B). This indicates that the amounts of cytochrome P450+cytochrome P420 determined spectrophotometrically correspond to the amounts of apoprotein and that, therefore, the endogenous heme sysnthesis in V79MZ cells is sufficient.

For a comparison of different cell lines and allelic variants either the amount of functional holoenzyme of cytochrome P450, the total amount of cytochrome P450+P420, the total amount of apoprotein or the level of expression, i.e. the mRNA content, may be used as a basis. Depending on the basis, the comparison may yield very different results, for example due to differences in heme incorporation or protein stability. In the following, the total amounts of cytochrome P450+P420 will be used as a basis. The corresponding values in Table 3 are printed in bold letters. For this purpose it was assumed that the amount of P420 depended substantially on the preparation since although the total amount of P420+P450 remained nearly constant the fraction of P420 varied between different preparations.

Comparison to Previous Measurements

By using a novel method of solubilization it was possible to record a CO difference spectrum with 100times less V79 cells than had to be used before. The amounts of cytochrome P450 determined in this manner were somewhat higher than those published previously for V79MZCYP cell lines (Table 4).

TABLE 4
Comparison of the amounts of cytochrome P450 determined by means
of CO difference spectra in V79MZCYP cell lines to previous results.

| | CO spectra with | | |
|---|---|---|---|
| | solubilizate | | |
| | pmol CYP/mg | | |
| | cellular protein | | |
| Cell line | P450 | P420 | Reference |
| V79MZ parental | 0 | 0 | 0 pmol/mg |
| | | | (Onderwater et al., 1996) |
| | | | microsomal CO difference |
| | | | spectrum |
| V79MZh1A1 | 11.4 ± 1.8 | 0 | 14 pmol/mg |
| | | | (Onderwater et al., 1996) |
| | | | microsomal CO difference |
| | | | spectrum |
| V79MZh3A4-hOR | 8.0 ± 1.0 | 3.0 ± 0.9 | 5 pmol/mg |
| | | | (Schneider et al., 1996) |
| | | | Western analysis of |
| | | | cell homogenate |

### Example 5

### Hydroxylation of Bufuralol

To confirm the functionality of cytochrome P450 2D6 expressed in a heterologous manner the hCYP2D6-specific hydroxylation of bufuralol (1′-hydroxylation of (+)-bufuralol or 4-hydroxylation of (−)-bufuralol) was determined (FIG. 15).

For this purpose, a diluted aliquot of cell homogenate was thawed on ice and resuspended carefully using a pipette. The reaction was started by addition of homogenate to the reaction sample (in the final sample: 100 μl total reaction volume, 150 μg total protein (V79MZh2D6*10: 300 μg), 200 μM bufuralol, 2 mM NADPH (Boehringer Mannheim GmbH, Mannheim) in 0.1 M potassium phosphate buffer, pH 7.4) and after an incubation in a water bath at 37° C. for 30 min (V79MZh2D6*10: 90 min) was stopped by addition of 12 μl 60% (v/v) perchloric acid (Boehringer Mannheim GmbH, Mannheim; approx. 0.33 M). The samples were incubated for several minutes on ice and the precipitate was collected by centrifugation (10 min, 17,000×g, 4° C.). The substrate bufuralol and the product hydroxy-bufuralol contained in the supernatant were separated by means of HPLC and detected by fluorometry (Kronbach et al., 1987; Kronbach, 1991). The chromatographic separation was performed in an isocratic manner using an aqueous-organic mobile phase (30% (v/v) acetonitrile (HPLC pure, Riedel-de Haen, Seelze), 40% (v/v) methanol, 30% (v/v) water, 2 mM perchloric acid) at a flow rate of 1 ml/min and 50° C. on a Hypersil ODS C18 “reversed phase” column (24 cm×4,6 mm, particle size 5 μm; Supelco, Bellefonte, Pa.). A C8 column was connected ahead of the system. The fluorescence signal (excitation at 252 nm, emission at 352 mn) was detected with a Fluorescence HPLC Monitor RF-530 (Shimadzu (Europe) GmbH, Düsseldorf), recorded by Chromatopac C-R3A 530 (Shimadzu (Europe) GmbH, Düsseldorf), and the peak area was integrated automatically.

The retention times were about 8 min for hydroxy-bufuralol and about 22 min for bufuralol. The quantification was carried out using a standard curve prepared from 1′-hydroxy-bufuralol standards in the range of 0.5-20 μM final concentrations after incubation with homogenate of the mock transfected cell line V79MZmockneo.

The concentrations of the stock solutions prepared gravimetrically were checked spectrophotometrically. Extinction coefficients of 16.3 mM⁻¹ cm⁻¹ for 1′-hydroxy-bufuralol at 245.5 nm (Gentest Corp., Woburn, Mass.) and 15.1 mM⁻¹ cm⁻¹ for bufuralol at 248 nm (Ultrafine Chemicals, London, England) were used in the calculations.

At 37° C. the reaction was in the linear range for 30 min up to 150 μg total protein/100 μl. To obtain a measurable signal in the case of variant hCYP2D6 10, 300 μg total protein/100 μl were incubated for 90 min. Therefore, the activities given for hCYP2D6 10 underestimate the real values; see Table 6.

Inhibition of the Bufuralol Hydroxylation

For inhibition studies, to the bufuralol hydroxylation reaction sample was added quinidine in a final concentration of 0.01-2 μM (2 mM stock solution in methanol, afterwards diluted in 0.1 M potassium phosphate buffer, pH 7.4; the assay contained <0.1% methanol; control without quinidine with 0.1% methanol) or inhibitory anti-hCYP2D6 antiserum (LKM serum 2) and human control serum (provided courteously by Dr. U. M. Zanger, Dr. Margarete Fischer-Bosch-Institut, Stuttgart) in final dilutions of 1:100-1:1000.

In all hCYP2D6 variants the hydroxylation of bufuralol was nearly completely inhibited with hCYP2D6-specific antiserum in a final dilution of 1:100. At a final concentration of 1 μM of the hCYP2D6-specific inhibitor quinidine the bufuralol hydroxylation was inhibited to about 90% independently of the allele (FIG. 16).

### Example 6

### Conditions for Culturing and Homogenization

To exclude that the endogenous heme synthesis is limiting for the amount of functional cytochrome P450, the culture medium was supplemented with hemin chloride (Sigma, Deisenhofen) or with the limiting synthesis precursor δ-aminolevulinic acid (hydrochloride, Sigma, Deisenhofen) and ferric (III) citrate (Sigma, Deisenhofen), respectively. Up to the cytotoxic limit at 10 μM for hemin chloride or 10 mM for 5-aminolevulinic acid/ferric (III) citrate, no increased bufuralol hydroxylase activity per mg total protein could be detected in the cell homogenate.

The bufuralol hydroxylase activity in the cell homogenate, however, was dependent on the cell density at the time of harvesting the cells, from the type of homogenization, and the number of freeze/thaw cycles. Therefore, the method for preparing the cell homogenate was optimized:

At cell densities of more than 90%, the bufuralol hydroxylase activity per mg total protein in the cell homogenate decreased by about 10-20%. In overgrown cultures with cell densities of clearly more than 100% only about half of the maximal activity was measured. To obtain cell homogenate for enzmyatic reactions the cells had to be disrupted. The highest activities were determined after freezing in liquid nitrogen. The use of a glas-on-glas homogenizer resulted in 10-20% lower activities. The loss in activity could be avoided by the addition of 1 mM PMSF as a protease inhibitor. Sonication either in the absence or presence of PMSF resulted in a loss of up to 70% of the activity. Repeated freezing and thawing of the cell homogenate led to non-reproducible variations in the bufuralol hydroxylase activity.

Optimal conditions for the preparation of cell homogenate are cell densities of 90%, followed by harvesting of the cells by centrifugation, resuspending in buffer, aliquoting, shock freezing in liquid nitrogen and storage at −80° C. until use. The aliquots were thawed only once and unused residues were discarded.

### Example 7

### Coexpression of hCYP2D6 and hCYPOR and Comparison of the Promoters

For some of the heterologously expressed cytochrome P450 isoforms the endogenous CYPOR synthesis in V79 cells is insufficient for maximal enzyme activity. For this reason, hCYP3A4 and hCYPOR, for example, were coexpressed (Schneider et al., 1996). A similar approach was followed with hCYP2D6 1 (transfection sample T11); see Table 5.

To detect the hCYPOR activity, the activity of the NADPH-dependent cytochrome c reductase was measured (Kubota et al., 1977). For this purpose, a diluted aliquot of cell homogenate was thawed on ice and resuspended carefully by means of a pipette. After preincubation of the reaction sample for 2 min at 37° C. (final sample: 800 μl total reaction volume, 40-100 μg total protein, 225 μM potassium cyanide (Sigma, Deisenhofen) to inhibit the reoxidation of reduced cytochrome c by cytochrome oxidase, 125 μM NADPH, 45 μM ferricytochrome c (Sigma, Deisenhofen) from bovine heart in 50 mM potassium phosphate buffer, pH 7.8) the reaction was started by addition of cytochrome c and the increase in the extinction at 550 nm and 37° C. was recorded using an Uvikam 941 Plus UV/VIS-spektrophotometer (Kontron Instruments Ltd., Watford, England). For the calculation of the cytochrome c reductase activity from the initial slope an extinction coefficient of 19.1 mM⁻¹ cm⁻¹ was used (Chance, 1957).

In accordance with the results of Schneider et al. (1996) the endogenous cytochrome c reductase activity of V79MZ cells was 9-14 nmol/min/mg cellular protein. Clearly higher cytochrome c reductase activities of 31-182 nmol/mg/min were measured if hCYPOR was coexpressed.

TABLE 5
Cytochrome c reductase activities of CYPOR (cyt_{c} red.) and bufuralol hydroxylase activities
of hCYP2D6. The selectivity is calculated by the ratio (−)-bufuralol activity/(+)-bufuralol
activity. The mean values and standard deviations of at least three independent
measurements are given.

| | | | | | | bufuralol hydroxylation | | | |
|---|---|---|---|---|---|---|---|---|---|
| | | | bufuralol hydroxylation | | | pmol/pmol | | | |
| | | cyt_{c} red. | pmol/mg/min | | | hCYP2D6/min | | | selectivity |
| Cell line | Promoter | nmol/mg/min | (+) | (+/−) | (−) | (+) | (+/−) | (−) | (−)/(+) |
| V79MZmockneo | — | 8.8 ± 1.3 | 0 | 0 | 0 | — | — | — | — |
| V79MZhOR | — | 124.6 ± 24.9 | — | — | — | — | — | — | — |
| V79MZh3A4- | — | 31.5 ± 8.5 | — | — | — | — | — | — | — |
| hOR | | | | | | | | | |
| V79MZh2D6*1- | SV40 | 182.2 ± 8.0 | 292 ± 34 | 143 ± 6 | 64 ± 6 | 7.0 ± 0.8 | 3.4 ± 0.2 | 1.5 ± 0.2 | 0.22 ± 0.05 |
| hOR | | | | | | | | | |
| V79MZh2D6*1 | SV40 | 13.5 ± 4.2 | 163 ± 32 | 108 ± 15 | 65 ± 14 | 6.5 ± 2.1 | 4.3 ± 1.2 | 2.6 ± 0.9 | 0.40 ± 0.16 |
| V79MZh2D6*1-H | CMVp | 14.1 ± 1.6 | 139 ± 27 | 76 ± 5 | 60 ± 13 | 6.9 ± 1.3 | 3.8 ± 0.3 | 2.9 ± 0.7 | 0.43 ± 0.18 |
| V79MZh2D6*1-S | MPSV- | 9.9 ± 1.1 | 110 ± 19 | 57 ± 5 | 45 ± 1 | 6.3 ± 1.7 | 3.3 ± 0.6 | 2.6 ± 0.3 | 0.41 ± 0.08 |
| | LTR + CMVe | | | | | | | | |

Abbreviations:
(+): (+)-bufuralol; (−): (−)-bufuralol; (+/−): racemic bufuralol; SV40: SV40 early promoter; CMVₚ: cytomegalovirus promoter; MPSV-LTR + CMVe: myeloproliferative sarcoma virus long terminal repeat and cytomegalovirus enhancer

If a coexpression of hCYP2D6 1 and hCYPOR was performed, markedly higher bufuralol hydroxylase activities were measured as in the case of a heterologous expression of hCYP2D6 1 alone (Table 5). After normalization of the amounts of hCYP2D6 (see Table 4) nearly identical reaction rates were obtained for all hCYP2D6 1-expressing cell lines independently of the coexpression of hCYPOR. Thus, in contrast to hCYP3A4 the activity of hCYP2D6 is not limited by the endogenous CYPOR activity. The differences in activity between the cell lines V79MZh2D6*1-hOR and V79MZh2D6*1 therefore are due to different integration of the cDNA expression cassette into the V79MZ genome. Furthermore, a comparison to the activities of cell lines V79MZh2D6*1-H and V79MZh2D6*1-S showed that the “integration effect” clearly has a greater influence on the differences in activity between different cell lines or homogenous clones of a transfection sample, respectively, than the promoter selected. Particularly in the V79 system comparable expression rates are obtained with the SV40 promoter and the CMV promoter while in COS-1 cells 10fold higher expression rates are obtained with the CMV promoter (Clark and Waterman, 1991). Similar results were obtained by Schneider et al. (1996).

The good agreement of the reaction rates further demonstrates the reliability of both the bufuralol hydroxylase activity test and the quantification of the amount of cytochrome P450 by means of CO difference spectra of solubilized cell homogenate.

### Example 8

### Comparison of Cell Lines V79MZh2D6*1, *2, *9, *10 and *17

Bufuralol Hydroxylase Activity

The polymorphic cell lines V79MZh2D6*1, *2, *9, *10 and *17 were compared with respect to the hCYP2D6-specific bufuralol hydroxylation. While the selectivity towards (+)-bufuralol was practically identical for all cell homogenates, the activities were decreased compared to the wildtype cell homogenate V79MZh2D6*1 (Table 6). Particularly the V79MZh2D6*10 cell homogenate exibited only about 2% of the activity of wildtype cell homogenate. After normalization of the amounts of hCYP2D6 (see Table 4) comparable reaction rates were obtained for hCYP2D6 1 and hCYP2D6 2 while the reaction rate of hCYP2D6 9 was about twice as high and the reaction rate of hCYP2D6 17 was about half as high.

TABLE 6
Bufuralol hydroxylase activities of the polymorphic cell lines at 200 μM bufuralol. The
selectivity is the ratio of (−)-bufuralol activity/(+)-bufuralol activity. The mean values and
standard deviations of at least three independent meansurements are given.

| | Bufuralol hydroxylase activity | | | bufuralol hydroxylase reaction rate | | | |
|---|---|---|---|---|---|---|---|
| | pmol/mg/min | | | pmol/pmol hCYP2D6/min | | | selectivity |
| Cell line | (+) | (+/−) | (−) | (+) | (+/−) | (−) | (−)/(+) |
| V79MZmockneo | 0 | 0 | 0 | — | — | — | — |
| V79MZh2D6*1 | 162.7 ± 31.6 | 108.0 ± 15.3 | 65.0 ± 13.5 | 6.53 ± 2.11 | 4.34 ± 1.17 | 2.61 ± 0.88 | 0.40 ± 0.16 |
| V79MZh2D6*2 | 95.2 ± 28.6 | 59.7 ± 13.9 | 43.8 ± 14.7 | 7.11 ± 4.63 | 4.46 ± 2.60 | 3.27 ± 2.24 | 0.46 ± 0.29 |
| V79MZh2D6*9 | 93.9 ± 11.5 | 61.8 ± 4.7 | 45.9 ± 3.1 | 14.67 ± 6.2 | 9.66 ± 3.60 | 7.17 ± 2.61 | 0.49 ± 0.16 |
| V79MZh2D6*10 | 3.3 ± 0.4 | 2.3 ± 0.2 | 1.4 ± 0.3 | 1.48 ± 0.29 | 1.03 ± 0.17 | 0.63 ± 0.18 | 0.42 ± 0.14 |
| V79MZh2D6*17 | 29.3 ± 1.9 | 21.6 ± 2.4 | 14.4 ± 3.3 | 3.57 ± 0.66 | 2.65 ± 0.61 | 1.77 ± 0.62 | 0.49 ± 0.14 |

Abbreviations:
(+): (+)-bufuralol; (−): (−)-bufuralol; (+/−): racemic bufuralol

Kinetic of the 1 -hydroxylation of (+)-bufuralol

The allele specific kinetic parameters of the 1′-hydroxylation of (+)-bufuralol were determined assuming a monophasic Michaelis-Menten kinetic without inhibitor (V=Vₘₐₓ* [S]/(K_{M}+[S]) (Table 7). The non-linear fitting of the curve to measuring points V was done by minimizing the sum of the error squares and weighting with a factor of 1/V (FIG. 17). The mean values and standard deviations of three independent measurements were used.

TABLE 7
Kinetic parameters of the 1′-hydroxylation of (+)-bufuralol for hCYP2D6 1, 2, 9, 10 and 17.
The mean values and standard deviations of three independent meansurements are given.

| | Based on mg of total protein | | | Based on the amount of cytochrome P450 | |
|---|---|---|---|---|---|
| | Vₘₐₓ | K_{M} | Clᵢₙₜ | reaction rate | Clᵢₙₜ |
| Cell line | pmol/mg/min | μM | ml/mg/min | pmol/pmol P450/min | ml/pmol P450/min |
| V79MZh2D6*1 | 170.8 ± 15.5 | 13.8 ± 1.6 | 12400 ± 2580 | 6.9 ± 1.5 | 500 ± 170 |
| V79MZh2D6*2 | 111.5 ± 2.1 | 22.6 ± 0.6 | 4940 ± 220 | 8.3 ± 3.1 | 370 ± 150 |
| V79MZh2D6*9 | 100.0 ± 15.0 | 15.0 ± 2.9 | 6670 ± 1970 | 15.6 ± 7.0 | 1040 ± 670 |
| V79MZh2D6*10 | 4.3 ± 0.1 | 53.3 ± 10.3 | 80 ± 17 | 1.9 ± 0.2 | 40 ± 10 |
| V79MZh2D6*17 | 34.3 ± 2.8 | 28.2 ± 1.3 | 1220 ± 160 | 4.2 ± 0.9 | 150 ± 40 |

Abbreviations:
Vₘₐₓ: maximal reaction rate at substrate saturation of the enzyme; K_{M}: Michaelis-Menten constant; Clᵢₙₜ: intrinsic clearance (Clᵢₙₜ = Vₘₐₓ/K_{M})

### Example 9

### Comparison to Other Expression Systems

hCYP2D6 2 (Substitutions Arg₂₉₆Cys and Ser₄₈₆Thr)

A slightly elevated K_{M} value and a comparable reaction rate as compared to the wildtype enzyme hCYP2D6 1 were determined for V79MZh2D6*2 homogenate. In contrast to wildtype enzyme, the CO difference spectrum shows low amounts of cytochrome P420.

If the cDNA expression was carried out in COS-1 cells the absolute bufuralol hydroxylase activity of hCYP2D6 2 compared to the wildtype was only about 60% at a similar reaction rate which is in complete agreement with the V79 expression system (Oscarson et al., 1997). It remained unclear whether this was just coincidence or whether an allele-dependent reason exisits. If hCYP2D6 1 and hCYP2D6 2 are expressed in yeast (Oscarson et al., 1997) the (+)-bufuralol reaction rate at substrate saturation was similar and the amount of holoprotein of hCYP2D6 2 was slightly decreased. It is discussed that this may be due to a decreased protein stability or also translation rate. In addition, also the peak of cytochrome P420 in the CO difference spectrum of V79MZh2D6*2 indicates a decreased protein stability compared to the wild type enzyme.

hCYP2D6 9 (Deletion of Lys₂₈₁)

An identical K_{M} value and a twice as high reaction rate compared to the wildtype enzyme hCYP2D6 1 were determined with V79MZh2D6*9 homogenate.

If hCYP2D6 9 was expressed in HepG2 cells using recombinant vaccinia virus and the (+)-bufuralol hydroxylation was examined, a 2.4fold higher K_{M} and an about 4fold higher reaction rate were measured compared to the wildtype enzyme hCYP2D6 1 (Tyndale et al., 1991a). I.e. the intrinsic clearance of hCYP2D6 9 was twice as high compared to the wildtype in both in vitro expression systems.

hCYP2D6 10 (Substitutions Pro₃₄Ser and Ser₄₈₆Thr)

A 4fold higher K_{M} and a 3.5fold lower reaction rate were determined with V79MZh2D6*10 homogenate as compared to the wildtype enzyme hCYP2D6 1. Thus, the intrinsic clearance was about one twelfth of that of hCYP2D6 1. The normation, however, was carried out using the cytochrome P420 content since no peak could be quantified at 450 nm. Moreover, the Western blots demonstrated that the amount of apoprotein was drastically decreased compared to all other variants. This means that the reaction rate based on functional holoenzyme may correspond to that of the wildtype while the activity in the homogenate is only 2.5% of the activity of V79MZh2D6*1 homogenate. Due to the low activity of the V79MZh2D6*10 homogenate the determination of the K_{M} constant is also uncertain.

In vitro expression experiments in COS-1 cells showed that substitution Ser₄₈₆Thr alone sligthly increases the amount of hCYP2D6 10 expressed as compared to the wildtype while the substitution Pro₃₄Ser results in drastically reduced amounts of protein and in a 40fold reduced activity. In the combination of both substitutions in hCYP2D6 10 the activity-lowering effect of the substitution Pro₃₄Ser far predominates (Johansson et al., 1994; Kagimoto et al., 1990). This is in close agreement to the findings of the Western analysis and the difference in activity beween the homogenates of V79MZh2D6*10 and V79MZh2D6*1. Thus, while 40fold differences in activity are found between hCYP2D6 1 and hCYP2D6 10 in vitro the difference in vivo is only one tenth (Droll et al., 1998).

hCYP2D6 17 (Substitutions Thr₁₀₇Ile, Arg₂₉₆Cys and Ser₄₈₆Thr)

A 2fold elevated K_{M} and a 2fold lower reaction rate as compared to the wildtype enzyme hCYP2D6 1 were determined for V79MZh2D6*17 homogenate. In contrast to the wildtype enzyme, the CO difference spectrum shows a fraction of cytochrome P420 of about 50%. If the cDNA expression was carried out in COS-1 cells the absolute bufuralol hydroxylase activity of hCYP2D6 17 was only about 20% compared to the wildtype which is in agreement with the V79 expression system (Oscarsson et al., 1997): As observed with the substitutions Arg₂₉₆Cys and Ser₄₈₆Thr, also the substitution Thr₁₀₇Ile had no substantial effect on the reaction rate of bufuralol hydroxylation. The substitution Thr₁₀₇Ile, however, resulted in elevated amounts of protein similar to the substitution Ser₄₈₆Thr, while the combination of all three substitutions in hCYP2D6 17 led to decreased amounts of protein if the cDNA was expressed in COS-1 cells. Decreased enzyme stability or translation rate is discussed as the reason. Also the high proportion of cytochrome P420 in the CO difference spectrum of V79MZh2D6*17 indicates a decreased protein stability in comparison to the wildtype enzyme.

If the cDNA was expressed in yeast, the reaction rate for bufuralol hydroxylation at substrate saturation was similar for all mutations and combinations (Oscarsson et al., 1997). The substitution of the hydrophilic Thr₁₀₇ by a hydrophic Ile alone in the conserved region of the β′ helix which also is a part of the first substrate recognition region, however, resulted in an elevated K_{M} for codeine O-demethylation (Oscarsson et al., 1997). In contrast, the K_{M} value for the hydroxylation of bufuralol remained unchanged. For this reaction, only a combination of the substitutions Thr₁₀₇Ile and Arg₂₉₆Cys resulted in a 5fold increase in K_{M}. Thus, hCYP2D6 17 is the only known variant of hCYP2D6 in which a combination of different substitutions is responsible for an altered affinity of the enzyme to the substrate.

### Example 10

### 4-hydroxylation of Tamoxifen by hCYP2D6

The cell lines according to the present invention were used to examine a possible effect of the hCYP2D6 polymorphism on the pharmacologically important 4-hydroxylation of tamoxifen (FIG. 18). Substrate and metabolites were separated and detected by means of HPLC/MSD (FIG. 19).

A diluted aliquot of cell homogenate was thawed on ice and carefully resuspended using a pipette. The reaction was started by addition of homogenate to the reaction sample (final sample: 100 μl total reaction volume, 150 μg total protein, 1-150 μM tamoxifen (2.5 μl stock solution in DMSO), 2 mM NADPH in 0.1 M potassium phosphate buffer, pH 7.4) and after an incubation for 30min (V79MZh2D6*10: 60 min) in a water bath at 37° C. was stopped by addition of 50 μl acetonitrile, 2% (v/v) acetic acid. The samples were incubated for several minutes on ice and the precipitate was collected by centrifugation (10 min, 17,000×g, 4° C.). The substrate tamoxifen and the reaction products including 4-hydroxy-tamoxifen contained in the supernatant were separated and detected by means of HPLC/ESI-MSD. The chromatographic separation was performed in a gradient of acetonitrile (FIG. 10) at a flow rate of 0.5 ml/min (12th to 19th minute 0.8 ml/min) and 30° C. using a C8 (2) “reversed phase” column (Luna, 150 mm×2 mm, particle size 5 um; Phenomenex, Hosbach). A C8 column (XDB-C8, narrow-bore column, 2.1 mm×12.5 mm; Zorbax HPLC Columns, Hewlett Packard, Waldbronn) was connected ahead of the system.

The detection of tamoxifen and its metabolites was performed using a HP Series 1100 MSD (Hewlett Packard, Waldbronn) in the single ion monitoring modus at m/z 344.2 (N-didemethyl-tamoxifen), m/z 358.3 (N-demethyl-tamoxifen), m/z 360.2 (monooxygenated metabolites of N-didemethyl-tamoxifen), mn/z 372.3 (tamoxifen), m/z 374.3 (monooxygenated metabolites of N-demethyl-tamoxifen), m/z 388.3 (monooxygenated metabolites of tamoxifen) and m/z 404.3 (monooxygenated metabolites of tamoxifen-N-oxide).

The retention times were 2.1 min for Z-tamoxifen-3,4-epoxide, approx. 5.5 and 6.2 min for E- and Z-4-hydroxy-tamoxifen, about 7.2 and 7.9 min for E- and Z-4-hydroxy-tamoxifen-N-oxide, about 8.4 min for Z-tamoxifen-1,2-epoxide, 9.9 min for Z-N-didemethyl-tamoxifen, 10.0 min for Z-N-demethyl-tamoxifen, 10.4 min for Z-tamoxifen-N-oxide and approx. 9.7 and 10.1 min for E- and Z-tamoxifen. The peaks of E- and Z-4-hydroxy-tamoxifen, Z-tamoxifen-N-oxide, Z-tamoxifen-1,2-epoxide, Z-N-demethyl-tamoxifen and Z-N-didemethyl-tamoxifen were verified and externally standardized using the corresponding pure substances. The calibration was performed for a final concentration range of 0.039-20 μM after incubation with homogenate of the mock transfected cell line of V79MZmockneo.

To identify the isoforms of cytochrome P450 involved in the 4-hydroxylation of tamoxifen incubations were performed with homogenates of cell lines V79MZh2EI and V79MZh3A4-hOR as well as with microsomes of insect cells coexpressing hCYP3A4 and hCYPOR and hCYP2C9*1 and hCYPOR, respectively, so-called “supersomes” (Gentest, Woburn, Mass., Produkt-Nr. P207 und P218). With respect to the incubations with V79MZh2D6 homogenate the following parameters were varied: 150 μg V79MZh2E1 homogenate were incubated up to 45 min, up to 500 μg V79MZh3A4-hOR homogenate and up to 25 μl hCYP3A4-hOR “supersomes” corresponding to 50 pmol hCYP3A4 were incubated up to 60 min with 100 μM magnesium chloride and 10 μM EDTA, and 12.5 μl hCYP2C9*1-hOR “supersomes” corresponding to 25 pmol hCYP2C9*1 were incubated for 30 min with 100 μM magnesium chloride and 10 μM EDTA.

The reaction at 37° C. was in a linear range for 30 min up to 150 μg of total protein/100 μl. To be able to measure a signal which could be quantified in the case of hCYP2D6 10, the reaction sample had to be incubated for 60 min. Therefore, the activities indicated sligthly underestimate the real values.

Effect of DMSO as a Solubilizing Agent for Tamoxifen

Since tamoxifen has a poor water-solubility, DMSO was added as solubilizing agent. Acetonitrile and methanol did not improve the solubility. In addition, they were not as suitable as DMSO due to other undesired properties.

Up to a DMSO concentration of 2.5% the tamoxifen-4-hydroxylase activity was only sligthly affected. At a concentration of 10% DMSO the tamoxifen-4-hydroxylase activity was only about 20% of that obtained at 2.5% (FIG. 20). On the other hand, the onset slope of the kinetic at 10% DMSO was linear up to about 75 μM tamoxifen while at 2.5% DMSO it was linear only up to 50 μM. This behaviour exactly correponded to the maximum solubility of tamoxifen at 10% and 2.5% DMSO, respectively. Accordingly, the lower slope of the kinetik was not caused by the enzyme kinetic but was only dependent on the solubility limit of tamoxifen. To be able to measure a signal of all hCYP2D6 variants which could be quantified, all other measurements were performed with 2.5% DMSO.

An slight decrease in hCYP2D6 activity up to 2.5% DMSO has also been published for the O-demethylation of dextromethorphane (Chauret et al., 1998; Hickman et al., 1998). In contrast, a drastic decrease already at low concentrations of DMSO was found for the (±)-bufuralol hydroxylase activity (Busby et al., 1999). Possibly, the inhibitory effect of the solvents is dependent on the substrate.

Kinetics of the 4-hydroxylation of Tamoxifen

The linearity in the kinetic of the hCYP2D6-catalyzed 4-hydroxylation of tamoxifen was not limited by enzyme kinetics but was dependent only on the solubility limit of tamoxifen. Therefore, it was impossible to determine the Vₘₐₓ and K_{M} indepedently of each other. Thus, assuming a monophasic Michaelis-Menten kinetic without inhibitor (V=Vₘₐₓ*[S]/(K_{M}+[S])) the intrinsic clearance (Clᵢₙₜ=Vₘₐₓ/K_{M}) was calculated from the linear onset slope ([S]→0) (Table 8). Fitting of the line to measuring points V was performed by linear regression (FIG. 21). An analogous kinetic was recorded for the 4-hydroxylation of tamoxifen by hCYP2C9 1 (not shown).

TABLE 8
Kinetic parameters of the 4-hydroxylation of tamoxifen for hCYP3A4,
hCYP2C9 1 and hCYP2D6 1, 2, 9, 10 and 17. The values for hCYP3A4
and hCYP2C9 are based on a single measurement series. All other
values are mean values and standard deviations of three independent
measurement series.

| | Based on mg of total protein | | Based on the amount of CYP450 | |
|---|---|---|---|---|
| | V_{50 μM} | | reaction rate_{50 μM} | |
| Cell line | pmol/mg/min | Clᵢₙₜ ml/mg/min | pmol/pmol/min | Clᵢₙₜ ml/pmol/min |
| V79MZmockneo | 0 | 0 | 0 | 0 |
| h3A4 (supers.) | approx. 1.95 | — | approx. 0.10 | — |
| h2C9 1 (supers.) | 24.08 | 482 | 0.96 | 19.3 |
| V79MZh2D6*1 | 58.3 ± 8.2 | 1166 ± 165 | 2.34 ± 0.63 | 46.8 ± 12.6 |
| V79MZh2D6*2 | 21.2 ± 3.3 | 424 ± 66 | 1.58 ± 0.80 | 31.6 ± 16.0 |
| V79MZh2D6*9 | 26.2 ± 6.5 | 524 ± 131 | 4.09 ± 2.23 | 81.9 ± 44.8 |
| V79MZh2D6*10 | 0.41 ± 0.06 | 8.2 ± 1.2 | 0.18 ± 0.04 | 3.7 ± 0.8 |
| V79MZh2D6*17 | 6.0 ± 1.3 | 119 ± 27 | 0.73 ± 0.25 | 14.6 ± 5.0 |

Abbreviations:
V_{50 μM}: reaction rate at 50 μM tamoxifen; Clᵢₙₜ: intrinsic clearance; supers.: “supersomes”

To date, no comparative data for the kinetics of the hCYP2D6-catalyzed tamoxifen-4-hydroxylation have been available. At a substrate concentration of 1 μM, tamoxifen-4-hydroxylation activities of 0.7-0.8 pmol/min/mg protein and of 1.1-3 pmol/min/mg protein were detected with human liver microsomes of poor metabolizers and extensive metabolizers, respectively (Crewe et al., 1997). A tamoxifen concentration of 1 μM is in the range of the plasma concentration of the therapeutical dosage (Buckley and Goa, 1989). At a substrate concentration of 18 μM, the activities were between 6 and 8 pmol/min/mg protein for poor metabolizers and between 12 and 25 pmol/min/mg protein for extensive metabolizers (Crewe et al., 1997). With 1.2 and 21 pmol/min/mg cellular protein, respectively, the activities of V79MZh2D6*1 homogenate were in the same ranges. As in the case of the bufuralol hydroxylase activity (see Table 11) an agreement in the activities between V79MZh2D6*1 cell homogenate and human liver microsomes was observed. The substrate dependence, however, of the tamoxifen-4-hydroxylase activity of recombinant hCYP2D6 was more pronounced as in the case of human liver microsomes. Furthermore, activities in the ranges of those of V79MZh2D6*2 and *9 homogenate and higher than those of V79MZh2D6*10 and *17 homogenate were measured in liver microsomes of poor metabolizers. Therefore, besides hCYP2D6 other isoforms with higher affinity to tamoxifen, possibly hCYP2C9, must be involved in tamoxifen-4-hydroxylation in vivo so that the effect of the hCYP2D6 polymorphism in vivo may be masked at the low therapeutic concentrations of tamoxifen. Nevertheless, the comparison between poor and extensive metabolizers shows that there is indeed a relationship between the rate of tamoxifen-4-hydroxylation and the hCYP2D6 phenotype.

The relative intrinsic clearances of the hCYP2D6 catalyzed hydroxylation of tamoxifen and bufuralol are identical (Table 9). The absolute values for the 1′-hydroxylation of (+)-bufuralol are about 10fold higher than those of the 4-hydroxylation of tamoxifen. This difference can be explained by the very different structures and binding of the two substrates in the active site of hCYP2D6 (see FIG. 24).

TABLE 9
Comparison of the intrinsic clearances of the hydroxylation of tamoxifen and bufuralol using
the novel polymorphic cell lines V79MZh2D6*1, *2, *9, *10 and *17.

| | | cell line V79MZh2D6 | | | | |
|---|---|---|---|---|---|---|
| | intrinsic clearance | *1 | *2 | *9 | *10 | *17 |
| 4-hydroxylation | [ml/min/mg protein] | 1166 ± 165 | 424 ± 66 | 524 ± 131 | 8.2 ± 1.2 | 119 ± 27 |
| of tamoxifen | [ml/min/pmol P450] | 47 ± 13 | 32 ± 16 | 82 ± 45 | 4 ± 1 | 15 ± 5 |
| 1′-hydroxylation | [ml/min/mg protein] | 12400 ± 2580 | 4940 ± 220 | 6670 ± 1970 | 80 ± 17 | 1220 ± 160 |
| of (+)-bufuralol | [ml/min/pmol P450] | 500 ± 170 | 370 ± 150 | 1040 ± 670 | 40 ± 10 | 150 ± 40 |

Isoforms Involved in the 4-hydroxylation of Tamoxifen

An involvement of the isoforms hCYP3A4, hCYP2C9 and hCYP2E1 besides hCYP2D6 in the 4-hydroxylation of tamoxifen is discussed (Crewe et al., 1997; Dehal and Kupfer, 1997; Styles et al., 1994). To check the ambiguous results and to get an indication on the hCYP2D6-catalyzed fraction of the total tamoxifen 4-hydroxylation, incubations were carried out with homogenates of the cell lines V79MZh2E1 and V79MZh3A4-hOR as well as hCYP3A4-hOR and hCYP2C9*1-hOR “supersomes” (FIG. 22). In this case, the conditions for the incubations were not optimized. Therefore, the values given should only be construed as guide values (Table 8).

The 4-hydroxylation of tamoxifen was catalyzed by isoforms hCYP2D6, hCYP2C9 and hCYP3A4. Metabolites having masses of 388.3 (monooxygenated metabolites, FIG. 22) and 358.3 (demethylated metabolites, not shown) were detected. As expected, the predominating metabolites were 4-hydroxy-tamoxifen in the incubation with hCYP2D6 and hCYP2C9 and N-demethyl-tamoxifen in the incubation with hCYP3A4. The 4-hydroxylation of tamoxifen by hCYP3A4, however, was negligible. In contrast to the other isoforms, the peak areas of E- and Z-4-hydroxy-tamoxifen were about identical and the peak at 2.2 min (tamoxifen-3,4-epoxide?) was strikingly high.

If cell line V79MZh2E1 was incubated with homogenate, no metabolites were detected which had not also been observed previously after incubation with homogenate of the mock transfected cell line V79Mzmockneo, i.e. tamoxifen-N-oxide and N-demethyl-tamoxifen. Both metabolites were contained as contaminations in tamoxifen though in a smaller amount than after the incubation.

Tamoxifen-N-oxide was detected in all samples independently of hCYP2D6 or other enzymes and also with heat-inactivated homogenate of mock-transfected cells. The amount was practically independent of the amount of total protein and the concentration of tamoxifen, and varied up to a factor of 20 between the different measurement series. The standard curve for tamoxifen-N-oxide for worked up standard solutions in negative control samples with tamoxifen was shifted in parallel as compared to standard solutions which had not been worked up. The reason for these findings was assumed to be a mere chemical oxidation of the dissolved tamoxifen, presumably by oxygen in air. Thus, the interval between incubation and sample measurement would provide a clue for the differing amounts in different measurement series.

N-Demethyl-tamoxifen was also detected in all samples independently of hCYP2D6 or other enzymes and also with heat-inactivated homogenate of mock-transfected cells. In contrast to tamoxifen-N-oxide, however, the amount of N-demethyl-tamoxifen depended both on the tamoxifen concentration and on the amount of total protein in the sample and varied only by a factor of 2 between different measurement series. It was impossible to carry out an exact quantification.

TABLE 10
Comparison of the contribution of different cytochrome P450 isoforms to the 4-
hydroxylation of tamoxifen in different expression systems.

| | | | Microsomes of recombinant human B | | | |
|---|---|---|---|---|---|---|
| | | “supersomes” | lymphoblastoid cells | | | human liver |
| | V79MZhCYP | of recombinant insect | (Dehal & Kupfer, | (Styles et al., | (Crewe et al., | microsomes |
| Isoform | homogenate | cells | 1997) | 1994) | 1997) | (Crewe et al., 1997) |
| hCYP2E1 | − | n.e. | − | + | − | − |
| hCYP3A4 | − | − | − | − | + | + |
| hCYP2C9*1 | n.e. | + | − | − | + | + |
| hCYP2D6*1 | + | n.e. | + | + | + | + |

+: unambiguous contribution to the 4-hydroxylation of tamoxifen;
−: no or only negligible tamoxifen 4-hydroxylation;
n.e.: not examined

Table 10 compares the results of the present and of previous studies. Considering all results, it seems that hCYP2E1 does not contribute to tamoxifen 4-hydroxylation. Whether tamoxifen-N-oxide and N-demethyl-tamoxifen were formed in the incubation with V79MZh2E1 as reported by Dehal and Kupfer (1997) remained unclear because of the high background of these two metabolites.

Human cytochrome P450 2D6 catalyzed the 4-hydroxylation of tamoxifen in all expression systems.

In contrast, the role of hCYP3A4 remained unclear: Although a substantial contribution of hCYP3A4 to the formation of 4-hydroxy-tamoxifen was found in inhibition studies in human liver microsomes (Crewe et al., 1997), the tamoxifen 4-hydroxylation was practically negligible in all other expression systems. In accordance with Dehal and Kupfer (1997) and Styles et al. (1994) both V79MZh3A4-hOR homogenate and hCYP3A4-hOR “supersomes” catalyzed the N-demethylation of tamoxifen.

Baculovirus-expressed hCYP2C9*1 (“supersomes”) clearly catalyzed the 4-hydroxylation of tamoxifen. The intrinsic clearance was about half that of hCYP2D6 1 expressed in V79MZ cells. With about 20% the fraction of hCYP2C9 on the total amount of cytochrome P450 in the liver, however, is about 10fold higher than that of hCYP2D6. Therefore, although the studies with microsomes of recombinant human B lymphoblastoid cells yielded ambiguous results a substantial contribution of hCYP2C9 to the 4-hydroxylation of tamoxifen in vivo may be assumed. Presumably, these discrepancies may be explained by different (concentrations of) solvent(s) during incubation.

Metabolites of Tamoxifen as Substrates of hCYP2D6

To examine the effect of certain modifications of the substrate, tamoxifen, and particularly of the nitrogen which is important for substrate binding, on hCYP2D6-catalyzed hydroxylation, incubations were carried out with various derivatives of tamoxifen. For all experimental series the relative reaction rates of the allelic variants were about the same. For this purpose, the relative peak areas were evaluated for non-standardized metabolites.

Configuration Isomers

In contrast to Z-tamoxifen, the configuration isomer E-tamoxifen was practically not metabolized by V79MZh2D6 homogenate.

Z-4-Hydroxy-tamoxifen

The predominant metabolite of the hCYP2D6-catalyzed tamoxifen metabolism, Z-4-hydroxy-tamoxifen, was metabolized by V79MZh2D6 homogenate. The metabolites were identified as dihydroxy derivatives with respect to their mass spectra. For example, the hCYP2D6-catalyzed ortho-hydroxylation of 4-hydroxy-tamoxifen forming the catechol has been described (Dehal and Kupfer, 1999). A detailed identification of the metabolites was impossible since no appropriate standards were available.

Although this reaction should be less important quantitatively due to the low in vivo concentration of 4-hydroxy-tamoxifen it is of interest in view of toxicology because of the formation of catechols which may form protein adducts (Dehal and Kupfer, 1999).

Modifications of the Tamoxifen Nitrogen

Z-Tamoxifen-N-oxide

Z-Tamoxifen-N-oxide was reacted to Z-4-hydroxy-tamoxifen-N-oxide, although only to a minor extent. A corresponding standard was synthesized by N-oxidation of 4-hydroxy-tamoxifen with hydrogen peroxide.

Z-N-Demethyl-tamoxifen

The reaction of Z-N-demethyl-tamoxifen proceeded well. The main metabolite had about the same retention time as Z-4-hydroxy-tamoxifen, and the chromatogram was similar to that obtained after incubation with Z-tamoxifen. Presumably, Z-4-hydroxy-N-demethyl-tamoxifen and all other metabolites typical for Z-tamoxifen were generated in the Z-N-demethyl form.

Z-N-Didemethyl-tamoxifen

The reaction of Z-N-didemethyl-tamoxifen was negligible.

### Example 11

### Comparison to Human Liver Microsomes and Purified Native hCYP2D6

The enzyme kinetic characteristics of recombinant hCYP2D6 were in the physiological range as demonstrated by a comparison to liver microsomes and purified native hCYP2D6 (Table 11). With 171±16 pmol/mg/min the (+)-bufuralol hydroxylase activity of V79MZh2D6*1 cell homogenate was in good agreement with published values for human liver microsomes of 167±43 (Kronbach et al., 1987) and 199±80 (Dayer et al., 1987), respectively.

TABLE 11
Comparison of the kinetic parameters of the hCYP2D6 1-catalyzed bufuralol hydroxylation
in different expression systems. If the evaluation was performed assuming a biphasic
Michaelis-Menten kinetic, the values for the isoform with high affinity and stereoselectivity
are given. The selectivity is the ratio of (−)-bufuralol activity/(+)-bufuralol activity.

| | | K_{M} | Vₘₐₓ (+) | | Reaction rate | | |
|---|---|---|---|---|---|---|---|
| Expression system | Work up | μM | pmol/mg/min | | 1/min | | selectivity |
| (Reference) | electron source | (+) | (+) | (+/−) | (+) | (+/−) | (−)/(+) |
| V79MZh2D6*1 | Homogenate | 13.8 ± 1.6 | 171 ± 16 | 108 ± 15 | 6.9 ± 1.5 | 4.3 ± 1.2 | 0.40 ± 0.16 |
| | NADPH | | | | | | |
| V79MZh2D6*1 | Homogenate | | | 153 ± 26 | | | |
| (Bogni, 1999) | NADPH | | | | | | |
| V79MZh2D6*1 | in culture | 7-8 | 170 ± 10 | | | | |
| (Appel, 1999) | | | | | | | |
| Human liver^{*1} | microsomes | 4.7 ± 2.2 | 167 ± 43 | | | | 0.56 ± 0.17 |
| (Kronbach et al., 1987) | NADPH | | | | | | |
| Human liver^{*2} | microsomes | 17.9 ± 6.3 | 199 ± 80 | | | | 0.49 ± 0.09 |
| (Dayer et al., 1987) | NADPH | | | | | | |
| Human liver^{*2} | microsomes | | 715 | | | | 0.46 |
| (Zanger et al., 1988) | NADPH | | | | | | |
| Human liver^{*1} | microsomes | | 50-2400 | | | | |
| (Gonzalez et al., 1988a) | NADPH | | | | | | |
| Human liver^{*2} | microsomes | 47.3 ± 8.1 | | | 0.6 ± 0.2 | | 0.48 ± 0.15 |
| (Gut et al., 1986) | NADPH | | | | | | |
| Human liver^{*1} | purified | 53.6 ± 27.4 | | | 3.4 ± 0.2 | | 0.15 ± 0.02 |
| (Gut et al., 1986) | CYPOR/NADPH | | | | | | |
| Human liver^{*1} | purified | 16.8 | 66333 | | 25.9 | | 0.17 |
| (Zanger et al., 1988) | CYPOR/NADPH | | | | | | |
| Human liver^{*1} | purified | | | | 3.7-9.5 | 4.4-6.4 | 0.14-0.16 |
| (Distlerath et al., 1985) | CYPOR/NADPH | | | | | | |
| E. coli | purified | 39 ± 5 | | | | 1.2 ± 0.1 | |
| (Gillam et al., 1995) | CYPOR/NADPH | (+/−) | | | | | |
| AHH-1 TK+/− | cell lysate | | 68 ± 0 | | | | |
| (2D6Met/Hol) | NADPH | | | | | | |
| (Crespi et al., 1991) | | | | | | | |
| AHH-1 TK+/− | cell lysate | | 126 ± 1 | | | | |
| (h2D6Metv2) | NADPH | | | | | | |
| (Penman et al., 1993) | | | | | | | |
| AHH-1 TK+/− | microsomes | 5.3 | 723 ± 35 | | 4.5 | | 0.42 |
| (h2D6Metv2) | NADPH | | | | | | |
| (Penman et al., 1993) | | | | | | | |
| AHH-1 TK+/− | microsomes | 6.7 ± 0.2 | | | 18.3 ± 0.1 | | |
| (h2D6Val/OR) | CYPOR/NADPH | | | | | | |
| (Crespi et al., 1995) | | | | | | | |
| AHH-1 TK+/− | microsomes | | | 550 | | 10.38 | |
| (h2D6Val/OR) | CYPOR/NADPH | | | | | | |
| (Gentest, produkt # | | | | | | | |
| M117r) | | | | | | | |
| COS-1 cells | homogenate | | 20-50 | | | | |
| (Kagimoto et al., 1990) | NADPH | | | | | | |
| COS-1 cells | homogenate | | 3.4 | | | | |
| (Johansson et al., 1994) | CYPOR/NADPH | | | | | | |
| Hep G2 | cell lysate | 4.0 | 34.1 | | 2.3 | | |
| (Tyndale et al., 1991a) | CuOOH | | | | | | |
| S. cerevisiae W(R) | microsomes | 2 | 116 | | 10 | | |
| (Oscarson et al., 1997) | Yred/NADPH | | | | | | |
| Sf9 insect cells | membranes | 18.5 ± 7.3 | 17500 ± 2700 | | 26.2 ± 0.4 | | 0.16 ± 0.02 |
| (Evert et al., 1997) | CYPOR/NADPH | | | | | | |
| Sf9 insect cells | membranes | 55 | 500-1000 | | 0.96 | | |
| (Patten et al., 1996) | CYPOR/NADPH | | | | | | |
| Sf9 insect cells | cell extract | 4.7 | | 370 | | 12.23 | |
| (Paine et al., 1996) | CYPOR/NADPH | (+/−) | | | | | |

Abbreviations:
(+): (+)-bufuralol; (−): (−)-bufuralol; (+/−): racemic bufuralol; CuOOH: cumene hydroperoxide; Yred: yeast reductase
^{*1}No indication of the hCYP2D6 genotype or phenotype.
^{*2}Phenotyped as EM using sparteine and/or debrisoquine in vivo.

In validation studies, a good reproducibility of the bufuralol hydroxylase activities for the polymorphic cell lines could be demonstrated (Table 11) although the experiments were performed according to different protocols and both in culture and in cell homogenate. Thus, the reproducibility and standardization of the novel cell lines was confirmed which is of critical importance for a future use in preclinical drug development.

With up to about 25 pmol/mg of cellular protein the amounts of cytochrome P450 in the V79MZh2D6 cell lines were in the physiological range of 8-115 pmol/mg in human liver microsomes (Distlerath et al., 1985). Similar values have been achieved in other mammalian cell systems such as COS-1 cells (Clark and Waterman, 1991) or human B lymphoblastoid cells (Crespi, 1991). With different expression systems such as baculovirus-infected insect cells substantially higher amounts of cytochrome P450 of up to 800 pmol/mg cell protein have been achieved (Evert et al., 1997). The level of expression, however, is only one criterion among many others in the selection of the suitable expression system. Substantially more important for most questions are the experimental possibilities provided by the expression system due to its biology.

References

Aklillu, E.; Persson, I.; Bertilsson, L.; Johansson, I.; Rodrigues, F.; Ingelman-Sundberg, M. (1996) Frequent distribution of ultrarapid metabolizers of debrisoquine in an Ethiopian population carrying duplicated and multiduplicated functional CYP2D6 alleles. J. Pharmacol. Exp. Ther. 278: 441-446.
Alvan, G. (1991) Clinical consequences of polymorphic drug oxidation. Fundam. Clin. Pharmacol. 5: 209-228.
Alvan, G.; Bechtel, P.; Iselius, L.; Gundert-Remy, U. (1990) Hydroxylation polymorphisms of debrisoquine and mephenytoin in European populations. Eur. J. Clin. Pharmacol. 39: 533-537.
Alvan, G.; Grind, M.; Graffner, C.; Sjoqvist, F. (1984) Relationship of N-demethylation of amiflamine and its metabolites to debrisoquine hydroxylation polymorphism. Clin. Pharmacol. Ther. 36: 515-519.
Aoyama, T.; Yamano, S.; Guzelian, P. S.; Gelboin, H. V.; Gonzalez, F. J. (1990) Five of 12 forms of vaccinia virus-expressed human hepatic cytochrome P450 metabolically activate aflatoxin B1. Proc. Natl. Acad. Sci. USA 87: 4790-4793.
Appel, K. (1999) PHARMBIODYN GmbH, Denzlingen. persönliche Mitteilung.
Armstrong, M.; Fairbrother, K.; Idle, J. R.; Daly, A. K. (1994) The cytochrome P450 CYP2D6 allelic variant CYP2D6J and related polymorphisms in a European population. Pharmacogenetics 4: 73-81.
Asseffa, A.; Smith, S. J.; Nagata, K.; Gillette, J.; Gelboin, H. V.; Gonzalez, F. J. (1989) Novel exogenous heme-dependent expression of mammalian cytochrome P450 using baculovirus. Arch. Biochem. Biophys. 274: 481-490.
Barnes, H. J.; Jenkins, C. M.; Waterman, M. R. (1994) Baculovirus expression of bovine cytochlrome P450c17 in Sf9 cells and comparison with expression in yeast, mammalian cells, and E. coli. Arch. Biochem. Biophys. 315: 489-494.
Becker, N. und Wahrendorf, J. (1981-1990). Brust. Krebsatlas der Bundesrepublik Deutschland. 3. Ausgabe. Heidelberg: Springer Verlag, 366-371.
Bertilsson, L. (1995) Geographical/interracial differences in polymorphic drug oxidation. Current state of knowledge of cytochromes P450 (CYP) 2D6 and 2C19. Clin. Pharmacokin. 29: 192-209.
Bertilsson, L.; Dahl, M. L.; Sjoqvist, F.; Aberg-Wistedt, A.; Humble, M.; Johansson, I.; Lundqvist, E.; Ingelman-Sundberg, M. (1993) Molecular basis for rational megaprescribing in ultrarapid hydroxylators of debrisoquine. The Lancet 341: 363.
Bertilsson, L.; Lou, Y. Q.; Du, Y. L.; Liu, Y.; Kuang, T. Y.; Liao, X. M.; Wang, K. Y.; Reviriego, J.; Iselius, L.; Sjoqvist, F. (1992) Pronounced differences between native Chinese and Swedish populations in the polymorphic hydroxylations of debrisoquin and S-mephenytoin. Clin. Pharmacol. Ther. 51: 388-397.
Bichara, N.; Ching, M. S.; Blake, C. L.; Ghabrial, H.; Smallwood, R. A. (1996) Propranolol hydroxylation and N-desisopropylation by cytochrome P4502D6. Drug Metab. Dispos. 24: 112-118.
Blech, J. (1999) Die ganz private Pille. Die Zeit 22: 42.
Bloomer, J. C.; Woods, F. R.; Haddock, R. E.; Lennard, M. S.; Tucker, G. T. (1992) The role of cytochrome P4502D6 in the metabolism of paroxetine by human liver microsomes. Clin. Pharmacol. 33: 521-523.
Bogni, A. (1999) European Centre for the Validation of Alternative Methods (ECVAM), Ispra, Italien. persönliche Mitteilung.
Boobis, A. R.; Murray, S.; Hampden, C. E.; Davies, D. S. (1985) Genetic polymorphism in drug oxidation: in vitro studies of human debrisoquine 4-hydroxylase and bufuralol 1′-hydroxylase activities. Biochem. Pharmacol. 34: 65-71.
Borgna, J.-L. and Rochefort, H. (1981) Hydroxylated metabolites of tamoxifene are formed in vivo and bound to estrogen receptor target tissues. J. Biol. Chem. 256: 859-868.
Bradley, M. O.; Bhuyan, B.; Francis, M. C.; Langenbach, R.; Peterson, A.; Huberman, E. (1981) Mutagenesis by chemical agents in V79 Chinese hamster cells. Mutat. Res. 87: 81-142.
Bradwell, L. (1989) The mutagenic and carcinogenic effects of gene transfer. Mutagenesis 4: 245-253.
Broly, F.; Marez, D.; Lo Guidice, J. M.; Sabbagh, N.; Legrand, M.; Boone, P.; Meyer, U. A. (1995a) A nonsense mutation in the cytochrome P450 CYP2D6 gene identified in a Caucasian with an enzyme deficiency. Hum. Genet. 96: 601-603.
Broly, F.; Marez, D.; Sabbagh, N.; Legrand, M.; Millecamps, S.; Lo Guidice, J.-M.; Boone, P.; Meyer, U. A. (1995b) An efficient strategy for detection of known and new mutations of the CYP2D6 gene using single strand conformation polymorphism analysis. Pharmacogenetics 5: 373-384.
Broly, F. and Meyer, U. A. (1993) Debrisoquine oxidation polymorphism: phenotypic consequences of a 3-base-paar deletion in exon 5 of the CYP2D6 gene. Pharmacogenetics 3: 123-130.
Brosen, K. and Gram, L. F. (1989a) Clinical significance of the sparteine/debrisoquine oxidation polymorphism. Eur. J. Clin. Pharmacol. 36: 537-547.
Brosen, K. and Gram, L. F. (1989b) Clinical significance of the sparteine/debrisoquine oxidation polymorphism. Eur. J. Clin. Pharmacol. 36: 537-547.
Brosen, K.; Otton, V.; Gram, L. F. (1986) Imipramine demethylation and hydroxylation: Impact of the sparteine oxidation phenotype. Clin. Pharmacol. Ther. 40: 543-549.
Buckley, M. T. and Goa, K. L. (1989) Tamoxifen: a reappraisal of its pharmacodynamic and pharmacokinetic properties, and therapeutic use. Clin. Pharmacol. 37: 451-490.
Burnette, W. N. (1981) Western-Blotting. Analyt. Biochem. 112: 195-203.
Busby, W. F. J.; Ackermann, J. M.; Crespi, C. L. (1999) Effect of methanol, ethanol, dimethyl sulfoxide, and acetonitrile on in vitro activities of cDNA-expressed human cytochromes P-450. Drug Metab. Dispos. 27: 246-249.
Buters, J. T. M.; Korzekwa, K. R.; Kunze, K. L.; Omata, Y.; Hardwick, J. P.; Gonzalez, F. J. (1994) cDNA-directed expression of human cytochrome P450 CYP3A4 using baculovirus. Drug Metab. Dispos. 22(5): 688-692.
Butner, K. A. and Lo, C. W. (1986) High frequency rearrangements associated with mouse centromeric satellite DNA. J. Mol. Biol. 187: 547-556.
Calos, M. P.; Lebkowski, J. S.; Botchan, M. R. (1983) High mutation frequency in DNA transfected into mammalian cells. Proc. Natl. Acad. Sci. USA 80: 3015-3019.
Carcillo, J. A.; Parise, R. A.; Adedoyin, A.; Frye, R.; Branch, R. A.; Romkes, M. (1996) CYP2D6 mRNA expression in circulating peripheral blood mononuclear cells correlates with in vivo debrisoquine hydroxylase activity in extensive metabolizers. Res. Commun. Mol. Pathol. Pharmacol. 91: 149-159.
Chance, B. (1957). Techniques for the Assay of the Respiratory Enzymes. In: Methods in Enzymology, Colowick, S. P. and Kaplan, N. O. (eds.). New York: Academic Press, IV: 273-329.
Chauret, N.; Gauthier, A.; Nicoll-Griffith, D. A. (1998) Effect of common solvents on in vitro cytochrome P450-mediated metabolic activities in human liver microsomes. Drug Metab. Dispos. 26: 1-4.
Chu, E. H. Y. and Malling, H. V. (1968) Mammalian cell genetics II.Chemical induction of specific locus mutations in Chinese hamster cells in vitro. Proc. Natl. Acad. Sci. 61: 1306-1312.
Clark, B. J. and Waterman, M. R. (1991). Heterologous expression of mammalian P450 in COS cells. In: Methods in Enzymology, Waterman, M. R. and Johnson, E. F., (eds.). New York: Academic Press, 206: 100-108.
Cooper, R. G.; Evans, D. A. P.; Price, A. H. (1987) Studies on the metabolism of perhexiline in man. Eur. J Clin. Pharmacol. 32: 569-576.
Crespi, C. L. (1991). Expression of cytochrome P450 cDNAs in human B lymphoblastoid cells: applications to toxicology and metabolite analysis. In: Methods in Enzymology, Waterman, M. R. and Johnson, E. F., (eds.). New York: Academic Press, 206: 123-129.
Crespi, C. L.; Penman, B. W.; Gelboin, H. V.; Gonzalez, F. J. (1991) A tobacco smoke-derived nitrosamine, 4-(methylnitrosamino)-1-(3-pyridyl)-1-butanone, is activated by multiple human cytochrome P450s including the polymorphic human cytochrome P4502D6. Carcinogenesis 12: 1197-1201.
Crespi, C. L.; Steimel, D. T.; Penman, B. W.; Korzekwa, K. R.; Femandezsalguero, P.; Buters, J. T. M.; Gelboin, H. V.; Gonzalez, F. J.; Idle, J. R.; Daly, A. K. (1995) Comparison of substrate metabolism by wild type CYP2D6 protein and a variant containing methionine, not valine, at position 374. Pharmacogenetics 5: 234-243.
Crewe, H. K.; Ellis, S. W.; Lennard, M. S.; Tucker, G. T. (1997) Variable contribution of cytochrome P450 2D6, 2C9 and 3A4 to the 4-hydroxylation of tamoxifen by human liver microsomes. Biochem. Pharmacol. 53: 171-178.
Dahl, M.; Johansson, I.; Bertilsson, L.; Ingelman-Sundberg, M.; Sjoqvist, F. (1995a) Ultrarapid hydroxylation of debrisoquine in a Swedish population. Analysis of the molecular genetic basis. J. Pharmacol. Exp.Ther. 274: 516-520.
Dahl, M. L. and Bertilsson, L. (1993) Genetically variable metabolism of antidepressants and neuroleptic drugs in man. Pharmacogenetics 3: 61-70.
Dahl, M. L.; Yue, Q. Y.; Roh, H. K.; Johansson, I.; Sawe, J.; Sjoqvist, F.; Bertilsson, L. (1995b) Genetic analysis of the CYP2D locus in relation to debrisoquine hydroxylation capacity in Korean, Japanese and Chinese subjects. Pharmacogenetics 5: 159-164.
Daly, A. K. (1995) Molecular basis of polymorphic drug metabolism. J. Mol. Med. 173: 539-553.
Daly, A. K.; Brockmoller, J.; Broly, F.; Eichelbaum, M.; Evans, W. E.; Gonzalez, F. J.; Huang, J.-D.; Idle, J. R.; Ingelman-Sundberg, M.; Ishizaki, T.; Jacqz-Aigrain, E.; Meyer, U. A.; Nebert, D. W.; Steen, V. M.; Wolf, C. R.; Zanger, U. M. (1996a) Nomenclature for human CYP2D6 alleles. Pharmacogenetics 6: 193-201.
Daly, A. K.; Fairbrother, K. S.; Andreasson, 0. A.; London, S. J.; Idle, J. R.; Steen, V. M. (1996b) Characterization and PCR-based detection of two different hybrid CYP2D7/CYP2D6 alleles associated with the poor metabolizer phenotype. Pharmacogenetics 6: 231-240.
Daly, A. K.; Leathart, J. B. S.; London, S. J.; Idle, J. R. (1995) An inactive cytochrome P450 CYP2D6 allele containing a deletion and a base substitution. Human Genet. 95: 337-341.
Dayer, P.; Kronbach, T.; Eichelbaum, M.; Meyer, U. A. (1987) Enzymatic basis of the debrisoquine/sparteine-type genetic polymorphism of drug oxidation: characterization of bufuralol 1′-hydroxylation in liver microsomes of in vivo phenotyped carriers of the genetic deficiency. Biochem. Pharmacol. 260: 4145-4152.
De Groene, E. M.; Hassing, I. G. M.; Blom, M. J.; Seinen, W.; Fink-Gremmels, J.; Horbach, G. J. (1996) Development of human cytochrome P450-expressing cell lines: application in mutagenicity of ochratoxin A. Cancer Res. 56: 299-304.
De Groot, M. J. and Vermeulen, N. P. E. (1997) Modeling the active sites of cytochrome P450s and glutathione S-transferases, two of the most important biotransformation enzymes. Drug Metab. Rev. 29: 747-799.
De Matteis, F.; White, I. N. H.; Smith, L. L. (1998) Species differences in the metabolic activation of tamoxifen into genotoxic derivatives: risk assessment in woman. Eur. J. Drug Metab. Pharmacokin. 23: 425-428.
Dehal, S. S. and Kupfer, D. (1996) Evidence that the catechol 3,4-dihydroxy tamoxifen is a proximate intermediate to the reactive species binding covalently to proteins. Cancer Res. 56: 1283-1290.
Dehal, S. S. and Kupfer, D. (1997) CYP2D6 catalyzes tamoxifen-4-hydroxylation in human liver. Cancer Res. 57: 3402-3406.
Dehal, S. S. and Kupfer, D. (1999) Cytochrome P-450 3A and 2D6 catalyze ortho hydroxylation of 4-hydroxytamoxifen and 3-hydroxytamoxifen (droloxifen) yielding tamoxifen catechol: involvement of catechols in covalent binding to hepatic proteins. Drug Metab. Dispos. 27: 681-688.
Distlerath, L. M.; Reilly, P. E. B.; Martin, M. V.; Davis, G. G.; Wilkinson, G. R.; Guengerich, F. P. (1985) Purification and characterization of the human liver cytochromes P-450 involved in debrisoquine 4-hydroxylation and phenacetin O-deethylation, two prototypes for genetic polymorphisms in oxidative drug metabolism. J. Biol. Chem. 260: 9057-9067.
Doehmer, J. (1993) V79 Chinese hamster cells genetically engineered for cytochrome P450 and their use in mutagenicity and metabolism studies. Toxicology 82: 105-118.
Doehmer, J.; Dogra, S.; Friedberg, T.; Monier, S.; Adesnik, M.; Glatt, H.; Oesch, F. (1988) Stable expression of rat cytochrome P-450IIB1 cDNA in Chinese hamster cells (V79) and metabolic activation of aflatoxin B₁. Proc. iVatl. Acad. Sci. USA 85: 5769-5773.
Doepker, C. L.; Livingston, G. K.; Schumann, B. L.; Srivastava, A. K. (1998) Structural and numerical chromosomal aberrations in a metabolically competent human lymphoblast cell line (MCL-5). Mutagenesis 13(3): 275-280.
Dogra, S.; Doehmer, J.; Glatt, H.; Molders, H.; Siegert, P.; Friedberg, T.; Seidel, A.; Oesch, F. (1990) Stable expression of rat cytochrome P-450IA1 cDNA in V79 Chinese hamster cells and their use in mutagenicity testing. Mol. Pharmacol. 37: 608-613.
Droll, K.; Bruce-Mensah, K.; Otton, S. V.; Gaedigk, A.; Sellers, E. M.; Tyndale, R. F. (1998) Comparison of three CYP2D6 probe substrates and genotype in Ghanians, Chinese and Caucasians. Pharmacogenetics 8: 325-333.
Eichelbaum, M.; Baur, M. P.; Dengler, H. J.; Osikowska-Evers, B. O.; Tieves, G.; Zekom, C.; Rittner, C. (1987) Chromosomal assignment of human cytochrome P-450 (debrisoquine/sparteine type) to chromosome 22. Br. J Clin. Pharmacol. 23: 455-458.
Eichelbaum, M.; Bertilsson, L.; Sawe, J.; Zekorn, C. (1982) Polymorphic oxidation of sparteine and debrisoquine: related pharmacogenetic entities. Clin. Pharmacol. Ther. 31: 184-186.
Eichelbaum, M. and Gross, A. S. (1990) The genetic polymorphism of debrisoquine/sparteine metabolism—clinical aspects. Pharmac. Ther. 46: 377-394.
Eichelbaum, M. and Gross, A. S. (1992). In: Pharmacogenetics of drug metabolism, Kalow, W., (ed.). New York: Pergamon Press, Inc..
Eichelbaum, M.; Spannbrucker, N.; Dengler, H. J. (1979a) Influence of the defective metabolism of sparteine on its pharrnacokinetics. Eur. J. Clin. Pharmacol. 16: 189-194.
Eichelbaum, M.; Spannbrucker, N.; Steincke, B.; Dengler, H. J. (1979b) Defective N-oxidation of sparteine in man: a new pharmacogenetic defect. Eur. J. Clin. Pharmacol. 16: 183-187.
Eichelbaum, M. and Woolhouse, N. M. (1985) Interethnic differences in sparteine oxidation among Ghanaians and Germans. Eur. J. Clin. Pharmacol. 28: 79-83.
Ellis, S. W.; Ching, M. S.; Watson, P. F.; Hendersson, C. J.; Simula, A. P.; Lennard, M. S.; Tucker, G. T.; Woods, H. F. (1992) Catalytic activities of human debrisoquine 4-hydroxylase cytochrome P450 (CYP2D6) expressed in yeast. Biochem. Pharmacol. 44: 617-620.
Ellis, S. W.; Hayhurst, G. P.; Smith, G.; Lightfoot, T.; Wong, M. M. S.; Simula, A. P.; Ackland, M. J.; Sternberg, M. J. E.; Lennard, M. S.; Tucker, G. T.; Wolf, C. R. (1995) Evidence that aspartic acid 301 is a critical substrate-contact residue in the active site of cytochrome P450 2D6. J. Biol. Chem. 270: 29055-29058.
Estabrook, R. W.; Peterson, J.; Baron, J.; Hildebrandt, A. (1972). The spectrophotometric measurement of turbid suspensions of cytochromes associated with drug metabolism. In: Methods in Pharmacology, Chigell, C. F., (ed.). New York (u.a.): Plenum Press, 2: 303-350.
Evans, D. A.; Mahgoub, A.; Sloan, T. P.; Idle, J. R.; Smith, R. L. (1980) A family and population study of the genetic polymorphism of debrisoquine oxidation in a white British population. J. Med Genet. 17: 102-105.
Evans, W. E.; Relling, M. V.; Rahman, A.; McLeod, H. L.; Scott, E. P.; Lin, J. S. (1993) Genetic basis for a lower prevalence of deficient CYP2D6 oxidative drug metabolism phenotypes in black Americans. J. Clin. Invest. 91: 2150-2154.
Evert, B.; Eichelbaum, M.; Haubruck, H.; Zanger, U. M. (1997) Functional properties of CYP2D6 1 (wildtype) and CYP2D6 7 (His324Pro) expressed by recombinant baculovirus in insect cells. Naunyn-Schmiedeberg's Arch. Pharmacol. 355: 309-318.
Evert, B.; Griese, E.-U.; Eichelbaum, M. (1994a) Cloning and sequencing of a new non-functional CYP2D6 allele: deletion of T1795 in exon 3 generates a premature stop codon. Pharmacogenetics 4: 271-274.
Evert, B.; Griese, E.-U.; Eichelbaum, M. (1994b) A missense mutation in exon 6 of the CYP2D6 gene leading to a histidine to proline exchange is associated with the poor metabolizer phaenotype of sparteine. Naunyn-Schmiedeberg's Arch. Pharmacol. 350: 434-439.
Fiers, W.; Contreras, R. R.; Haegeman, G.; Rogiers, R.; De Voorde, A.; van Heuverswyn, H.; Van Herreweghe, J.; G., V.; M., Y. (1978) Complete nucleotide sequence of SV40 DNA. Nature 273: 113-120.
Fischer, V.; Vogels, B.; Maurer, G.; Tynes, R. E. (1992) The antipsychotic clozapine is metabolized by the polymorphic human microsomal and recombinant cytochrome P450 2D6. J. Pharmacol. Exp. Ther. 260: 1355-1360.
Fonne-Fister, R.; Bargetzi, M. J.; Meyer, U. A. (1987) MPTP, the neurotoxin inducing Parkinson's disease, is a potent competitive inhibitor of human and rat cytochrome P450 isozymes (P450bufI, P450dbl) catalyzing debrisoquine 4-hydroxylation. Biochem. Biophys. Res. 148: 1144-1150.
Ford, D. K. and Yerganian, G. (1958) Observations on the chromosomes of Chinese hamster cells in tissue culture. J. Natl. Cancer Inst. 21: 393-425.
Furr, B. J. A. and Jordan, V. C. (1984) The pharmacology and clinical uses of tamoxifen. Pharmac. Ther. 25: 127-205.
Gaedigk, A.; Blum, M.; Gaedigk, R.; Eichelbaum, M.; Meyer, U. A. (1991) Deletion of the entire cytochrome P450 gene as a cause of impaired drug metabolism on poor metabolizers of the debrisoquine/sparteine polymorphism. Am. J. Hum. Genet. 48: 943-950.
Garfinkel, D. (1958) Isolation and properties of cytochrome b5 from pig liver. Arch. Biochem. Biophys. 77: 111-120.
Gillam, E. M. J.; Guo, Z.; Martin, M. V. M.; Jenkins, C. M.; Guengerich, F. P. (1995) Expression of cytochrome P450 2D6 in Escherichia coli, purification, and spectral and catalytic characterization. Arch. Biochem. Biophys. 319: 540-550.
Glatt, H.; Davis, W.; Meinl, W.; Hermersdorfer, H.; Venitt, S.; Phillips, D. H. (1998) Rat, but not human, sulfotransferase activates a tamoxifen metabolite to produce DNA adducts and gene mutations in bacteria and mammalian cells in culture. Carcinogenesis 19: 1709-1713.
Glatt, H.; Gemperlein, I.; Turchi, G.; Heinritz, H.; Doehmer, J.; Oesch, F. (1987) Search for cell culture systems with diverse xenobiotic-metabolizing activities and their use in toxicological studies. Mol. Toxicol. 1: 313-334.
Glatt, H. R.; Gemperlein, I.; Setiabudi, F.; Platt, K. L.; Oesch, F. (1990) Expression of xenobiotic metabolizing enzymes in propagable cell cultures and induction of micronuclei by 13 compounds. Mutagenesis 5: 241-249.
Gonzalez, F. J. and Korzekwa, K. R. (1995) Cytochromes P450 expression systems. Annu. Rev. Pharmocol. Toxicol. 35: 369-390.
Gonzalez, F. J.; Matsunaga, T.; Nagata, K.; Meyer, U.; Nebert, D. W.; Pastewka, J.; Kozak, C. A.; Gillette, J.; Gelboin, H. V.; Hardwick, J. P. (1987) Debrisoquine 4-hydroxylase: characterization of a new P450 gene subfamily, regulation, chromosomal mapping, and molecular analysis in the DA rat polymorphism. DNA 6: 149-161.
Gonzalez, F. J.; Skoda, R. C.; Kimura, S.; Umeno, M.; Zanger, U. M.; Nebert, D. W.; Gelboin, H. V.; Hardwick, J. P.; Meyer, U. A. (1988a) Characterization of the common genetic defect in humans deficient in debrisoquine metabolism. Nature 331: 442-446.
Gonzalez, F. J.; Skoda, R. C.; Kimura, S.; Umeno, M.; Zanger, U. M.; Nebert, D. W.; Gelboin, H. V.; Hardwick, J. P.; Meyer, U. A. (1990) Molecular characterization of the common human deficiency in metabolism of debrisoquine and other drugs. Nature 331: 442-446.
Gonzalez, F. J.; Vilbois, F.; Hardwick, J. P.; McBride, O. W.; Nebert, D. W.; Gelboin, H. V.; Meyer, U. A. (1988b) Human debrisoquine 4-hydroxylase (P4501ID6): cDNA and deduced amino acid sequence and assignment of the CYP2D6 locus to chromosome 22. Genomics 2: 174-179.
Gotoh, O. (1991) Substrate recognition sites in cytochrome P450 family 2 (CYP2). Proteins inferred from comparative analysis of amino acid and encoding nucleotide sequences. J. Biol. Chem. 267: 83-90.
Gough, A. C.; Miles, J. S.; Spurr, N. K.; Moss, J. E.; Gaedigk, A.; Eichelbaum, M.; Wolf, C. R. (1990) Identification of the primary gene defect at the cytochrome P450 CYP2D6 locus. Nature 347: 773-776.
Gough, A. C.; Smith, C. A.; Howell, S. M.; Wolf, C. R.; Bryant, S. P.; Spurr, N. K. (1993) Localization of the CYP2D gene locus to human chromosome 22q13.1 by polymerase chain reaction, in situ hybridization, and linkage analysis. Genomics 15: 430-432.
Graham, F. L. and Van der Eb, A. J. (1973) A new technique for the assay of infectivity of human adenovirus 5 DNA. Virology 52: 456-467.
Greim, H. und Deml, E. H. (Hrsg.) (1996). Toxikologie. Eine Einführung für Naturwissenschaftler und Mediziner. Weinheim: VCH.
Griese, E.-U.; Zanger, U. M.; Brudermanns, U.; Gaedigk, A.; Mikus, G.; Morike, K.; Stuven, T.; Eichelbaum, M. (1998) Assessment of the predictive power of genotypes for the in-vivo catalytic function of CYP2D6 in a German population. Pharmacogenetics 8: 15-26.
Gross, A. S.; Phillips, A. C.; Rieutord, A.; Shenfield, G. M. (1996) The influence of the sparteine/debrisoquine genetic polymorphism on the disposition of dexfenfluramine. Br. J. Clin. Pharmacol. 41: 311-317.
Guidice, J. M.; Marez, D.; Sabbagh, N.; Legrand-Andreoletti, M.; Spire, C.; Alcaide, E.; Lifitte, J. J.; Broly, F. (1997) Evidence for CYP2D6 expression in human lung. Biochem. Biophys. Res. 241: 79-85.
Gut, J.; Catin, T.; Dayer, P.; Kronbach, T.; Zanger, U.; Meyer, U. A. (1986) Debrisoquine/sparteine-type polymorphism of drug oxidation: purification and characterization of two functionally different human liver cytochrome P-450 isoenzymes involved in impaired hydroxylation of the prototype substrate bufuralol. J. Biol. Chem. 261: 11734-11743.
Halliday, R. C.; Jones, B. C.; Park, B. K.; Smith, D. A. (1997) Synthetic strategies to lower affinity for CYP2D6. Eur. J Drug Metab. Pharmacokin. 22: 291-294.
Hickman, D.; Wang, J.-P.; Wang, Y.; Unadkat, J. D. (1998) Evaluation of the selectivity of in vitro probes and suitability of organic solvents for the measurement of human cytochrome P450 monooxygenase activities. Drug Metab. Dispos. 26: 207-215.
Higashi, Y.; Hiromasa, T.; Tanae, A.; Miki, T.; Nakura, J.; Kondo, T.; Ohura, T.; Ogawa, E.; Nakayama, K.; Fujii-Kuriyama, Y. (1991) Effects of individual mutations in the P-450(C21) pseudogene on the P-450(C21) activity and their distribution in the patient genomes of congenital steroid 21-hydroxylase deficiency. J. Biochem. (Tokyo) 109: 638-644.
Hiroi, T.; Imaoka, S.; Funae, Y. (1998) Dopamine formation from tyramine by CYP2D6. Biochem. Biophys. Res. Commun. 249: 838-843.
Hogstedt, S.; Lindberg, B.; Rane, A. (1983) Increased oral clearance of metoprolol in pregnancy. Eur. J. Clin. Pharmacol. 24: 217-220.
Horai, Y.; Nakano, M.; Ishizaki, T.; Ishikawa, K.; Zhou, H. H.; Zhou, B. I.; Liao, C. L.; Zhang, L. M. (1989) Metoprolol and mephenytoin oxidation polymorphisms in Far Eastern Oriental subjects: Japanese versus mainland Chinese. Clin. Pharmacol. Ther. 46: 198-207.
Huang, Z.; Fasco, M. J.; Figge, H. L.; Keyomarsi, K.; Kaminsky, L. S. (1996) Expression of cytochromes P450 in human breast tissue and tumors. Drug Metab. Dispos. 24: 899-905.
Huang, Z.; Fasco, M. J.; Kaminsky, L. S. (1997) Alternative splicing of CYP2D mRNA in human breast tissue. Arch. Biochem. Biophys. 343: 101-108.
IARC (1996). Some pharmaceutical drugs: anti-estrogenic compounds. Tamoxifen. IARC monographs on the evaluation of carcinogenic risks to humans. WHO, 66: 253-365.
Ingelman-Sundberg, M.; Oscarson, M.; Persson, I.; Masimirembwa, C. M.; Dahl, M.-L.; Bertilsson, L.; Sjoqvist, F.; Johansson, I. (1995). Genetic polymorphism of human drug metabolizing enzymes. Recent aspects on polymorphic forms of cytochromes P450. European conference on specificity and variability in drug metabolism, Luxembourg, EU.
Jaenisch, R. and Jahner, D. (1984) Methylation, expression and chromosomal position of genes in mammals. Biochem. Biophys. Acta 782: 179-189.
Johansson, I.; Lundqvist, E.; Bertilsson, L.; Dahl, M.-L.; Sjoqvist, F.; Ingelman-Sundberg, M. (1993) Inherited amplification of an active gene in the cytochrome P450 CYP2D locus as a cause of ultrarapid metabolism of debrisoquine. Proc. Natl. Acad. Sci. USA 90: 11825-11829.
Johansson, I.; Oscarson, M.; Yue, Q.-Y.; Bertilsson, L.; Sjoqvist, F.; Ingelman-Sundberg, M. (1994) Genetic analysis of the Chinese cytochrome P4502D locus: characterization of variant CYP2D6 genes present in subjects with diminished capacity for debrisoquine hydroxylation. Mol. Pharmacol. 46: 452-459.
Jordan, V. C. (1997) Tamoxifen: the herald of a new era of preventive therapeutics. J. Nat. Cancer Inst. 89: 747-749.
Jordan, V. C. (1998) Designer estrogens. Scientific American Okt.: 60-67.
Jordan, V. C. and Allen, K. E. (1980) Evaluation of the antitumor activity of the non-steroidal anti-estrogen monohydroxytamoxifen in the DMBA-induced rat carcinoma model. Eur. J. Cancer Clin. Oncol. 16: 239-251.
Jordan, V. C. and Chem, C. (1982) Metabolites of tamoxifen in animals and man: identification, pharmacology, and significance. Breast Cancer Res. and Treatment 2: 123-138.
Jordan, V. C. and Robinson, S. P. (1987) Species-specific pharmacology of antiestrogens: role of metabolism. Federation proceedings 46: 1870-1874.
Kagimoto, M.; Heim, M.; Kagimoto, K.; Zeugin, T.; Meyer, U. A. (1990) Multiple mutations of the human cytochrome IID6 gene (CYP2D6) in poor metabolisers of debrisoquine. J. Biol. Chem. 265: 17209-17214.
Kempf, A. C.; Zanger, U. M.; Meyer, U. A. (1995) Truncated human P450 2D6: expression in Escherichia coli, Ni(2+)-chelate affinity purification, and characterization of solubility and aggregation. Arch. Biochem. Biophys. 321: 277-288.
Kiefer, F. and Wiebel, F. J. (1989) V79 Chinese hamster cells express cytochrome P-450 activity after simultaneous exposure to polycyclic aromatic hydrocarbons and aminophylline. Toxicol. Letters 48: 265-273.
Kimura, S.; Umeno, M.; Skoda, R. C.; Meyer, U. A.; Gonzalez, F. J. (1989) The human debrisoquine 4-hydroxylase (CYP2D) locus: sequence and identification of the polymorphic CYP2D6 gene, a related gene, and a pseudogene. Am. J. Hum. Genet. 45: 889-904.
Kivisto, K. T.; Griese, E.-U.; Stuven, T.; Fritz, P.; Friedel, G.; Kroemer, H. K.; Zanger, U. M. (1997) Analysis of CYP2D6 expression in human lung: implications for the association between CYP2D6 activity and susceptibility to lung cancer. Pharmacogenetics 7: 295-302.
Klingenberg, M. (1958) Pigments of rat liver microsomes. Arch. Biochem. Biophys. 75: 376-386.
Kozak, M. (1987) At least six nucleotides preceding the AUG initiator codon enhance translation in mammalian cells. J. Mol. Biol. 196: 947-950.
Kozak, M. (1990) Downstream secondary structure facilitates recognition of initiator codons by eukaryotic ribosomes. Proc. Natl. Acad .Sci. USA(87): 8301-8305.
Kroemer, H. K. and Eichelbaum, M. (1995) “It's the genes, stupid”. Molecular bases and clinical consequences of genetic cytochrome P450 2D6 polymorphism. Life Sci. 56: 2285-2298.
Kronbach, T. (1991) Bufuralol, dextromethorphan, and debrisoquine as prototype substrates for human P450IID6. Methods in Enzymology 206: 509-517.
Kronbach, T.; Mathys, D.; Gut, J.; Catin, T.; Meyer, U. A. (1987) High-performance liquid chromatographic assay fot bufuralol 1′-hydroxylase, debrisoquine 4-hydroxylase, and dextromethorphan O-demethylation in microsomes and purified cytochrome P-450 isozymes of human liver. Analytical Biochemistry 162: 24-32.
Krynetski, E. Y.; Drutsa, V. L.; Kovaleva, I. E.; Luzikov, V. N. (1995) High yield expression of functionally active human liver CYP2D6 in yeast cells. Pharmacogenetics 5: 103-109.
Krynetski, E. Y.; Drutsa, V. L.; Kovaleva, I. E.; Luzikov, V. N.; Uvarov, V. Y. (1993) Effects of amino-terminus truncation in human cytochrome P450IID6 on its insertion into the endoplasmatic reticulum membrane of Saccharomyces cerevisiae. FEBS Lett. 336: 87-89.
Kubota, S.; Yoshida, Y.; Kumaoka, H.; Furumichi, A. (1977) Studies on the microsomal electron-transport system of anaerobically grown yeast. J. Biochem. 81: 197-205.
Laemmli, U. K. (1970) Cleavage of structural proteins during the assembly of the head of bacteriophage T4. Nature 227: 680-685.
Lazarou, J.; Pomeranz, B. H.; Corey, P. N. (1998) Incidence of adverse drug reactions in hospitalized patients. JAMA 4 279: 1200-1205.
Lewis, D. F. V. (1998) The CYP2 family: models, mutants and interactions. Xenobiotica 28: 617-661.
Lim, C. K.; Yuan, Z.-X.; Lamb, J. H.; White, I. N. H.; De Matteis, F.; Smith, L. L. (1994) A comparative study of tamoxifen metabolism in female rat, mouse and human liver microsomes. Carcinogenesis 15: 589-593.
Lodwick, R.; McConkey, B.; Brown, A. M.; Beeley, L. (1987) Life threatening interaction between tamoxifen and warfarin. Br. Med. J. 295: 1141.
Logan, W. P. D. (1975) World Health Organisation Statistics Report. WHO, 28: 232.
Lowry, O. H.; Rosebrough, A. L.; Farr, A. L.; Randell, R. J. (1951) Protein measurement with the folin phenol reagent. J. Biol. Chem. 214: 741.
Lundqvist, E.; Johansson, I.; Ingelman-Sundberg, M. (1999) Genetic mechanisms for duplication and multiplication of the human CYP2D6 gene and methods for detection of duplicated CYP2D6 genes. Gene 226: 327-338.
MacGregor, J. and Jordan, V. C. (1998) Basic guide to the mechanisms of antiestrogen action. Pharmacol. Rev. 50: 151-196.
Mahgoub, A.; Idle, J. R.; Dring, L. G.; Lancaster, R.; Smith, R. L. (1977) Polymorphic hydroxylation of debrisoquine in man. Lancet 2: 584-586.
Marez, D.; Legrand, M.; Sabbagh, N.; Lo Guidice, J. M.; Boone, P.; Broly, F. (1996) An additional allelic variant of the CYP2D6 gene causing impaired metabolism of sparteine. Human Genetics 97: 668-670.
Marez, D.; Sabbagh, N.; Legrand, M.; Lo Guidice, J. M.; Boone, P.; Broly, F. (1995) A novel CYP2D6 allele with an abolished splice recognition site associated with the poor metabolizer phenotype. Pharmacogenetics 5: 305-311.
Marquardt, H. und Schäfer, S. G. (Hrsg.) (1997). Lehrbuch der Toxikologie. 1. Ausgabe. Heidelberg, Berlin: Spektrum Akademischer Verlag.
Masimirembwa, C.; Hasler, J.; Bertilssons, L.; Johansson, I.; Ekberg, O.; Ingelman-Sundberg, M. (1996a) Phenotype and genotype analysis of debrisoquine hydroxylase (CYP2D6) in a black Zimbabwean polulation. Reduced enzyme activity and evaluation of metabolic correlation of CYP2D6 probe drugs. Eur. J. Clin. Pharmacol. 51: 117-122.
Masimirembwa, C.; Persson, I.; Bertilsson, L.; Hasler, J.; Ingelman-Sundberg, M. (1996b) A novel mutant variant of the CYP2D6 gene (CYP2D6*17) common in black African population: association with diminished debrisoquine hydroxylase activity. Br. J. Clin. Pharmacol. 42: 713-719.
Matsunaga, E.; Umeno, M.; Gonzalez, F. J. (1990) The rat P450 IID subfamily: complete sequences of four closely linked genes and evidence that gene conversions maintained sequence homogeneity at the heme-binding region of the cytochrome P450 active site. J. Mol. Evol. 30: 155-169.
Meyer, U. A. (1991) Genotype or phenotype: the definition of a pharmacogenetic polymorphism. Pharmacogenetics 1: 66-67.
Meyer, U. A. and Zanger, U. M. (1997) Molecular mechanisms of genetic polymorphisms of drug metabolising enzymes. Annu. Rev. Pharmacol. Toxicol. 37: 269-296.
Monier, S.; Van Luc, P.; Adesnik, M. (1988) Signals for the incorporation and orientation of cytochrome P450 in the endoplasmatic reticulum membrane. J. Cell Biol. 107: 457-570.
Moorthy, B.; Sriram, P.; Pathak, D. N.; Bodell, W. J.; Randerath, K. (1996) Tamoxifen metabolic activation: comparison of DNA adducts formed by microsomal and chemical activation of tamoxifen and 4-hydroxy-tamoxifen with DNA adducts formed in vivo. Cancer Res. 56: 53-57.
Mortimer, O.; Persson, K.; Ladona, M. G.; Spalding, D.; Zanger, U. M.; Meyer, U. A.; Rane, A. (1990) Polymorphic formation of morphine from codeine in poor and extensive metabolizers of dextromethorphan: relationship to the presence of immunoidentified cytochrome P-450IID1. Clin. Pharmacol. Ther. 47: 27-35.
Mulligan, R. C. and Berg, P. (1981) Selection for animal cells that express the Escherichia coli gene coding for xanthine-guanine phosphoribosyltransferase. Proc. Natl. Acad. Sci. USA 78: 2972-2976.
Nayfield, S. G. (1995) Tamoxifen's role in chemoprevention of breast cancer: an update. J. Cell. Biol. Suppl. 22: 42-50.
Nebert, D. W. (1997) Polymorphisms in drug-metabolizing enzymes: what is their clinical significance and why do they exist? Am. J. Hum. Genet. 60: 265-271.
Nebert, D. W. and McKinnon, R. A. (1994) Cytochrome P450: evolution and functional diversity. Prog. Liv. Dis. 12: 63-97.
Nelson, D. R.; Koymans, L.; Kamataki, T.; Stegeman, J. J.; Feyereisen, R.; Waxman, D. J.; Gotoh, O., Coon, M. J.; Estabrook, R. W.; Gunsalus, I. C.; Nebert, D. W. (1996) P450 superfamily: update on new sequences, gene mapping, accession numbers and nomenclature. Pharmacogenetics 6: 1-42.
Nelson, D. R. and Stobel, H. W. (1988) On the membrane topolgy of vertebrate cytochrome P-450 proteins. J. Biol. Chem. 263: 6038-6050.
Oates, N. S.; Shah, R. R.; Idle, J. R.; Smith, R. L. (1982) Genetic polymorphism of phenformin 4-hydroxy-lation. Clin. Pharmacol. Ther. 32: 81-89.
Ohgiya, S.; Komori, M.; Ohi, H.; Shiramatsu, K.; Shinriki, N.; Kamataki, T. (1992) Six-base deletion occurring in messages of human cytochrome P-450 in the CYP2C subfamily results in reduction of tolbutamide hydroxylase activity. Biochemistry International 27: 1073-1081.
Omura, T. and Sato, R. (1964a) The carbon monoxide-binding pigment of liver microsomes. I. Evidence for its hemoprotein nature. J. Biol. Chem. 239: 2370-2385.
Omura, T. and Sato, R. (1964b) The carbon monoxide-binding pigment of liver microsomes. II. Solubilization, purification, and properties. J. Biol. Chem. 239: 2379-2385.
Onderwater, R. C. A.; Goeptar, A. R.; Levering, P. R.; Vos, R. M. E.; Konings, P. N. M.; Doehmer, J.; Commandeur, J. N. M.; Vermeulen, N. P. E. (1996) The use of macroporous microcarriers for the large-scale growth of V79 cells genetically designed to express single human cytochrome P450 isoenzymes and for the characterization of the expressed cytochrome P450. Prot. Expr. Pur. 8: 439-446.
Osborne, C. K. (1998) Tamoxifen in the treatment of breast cancer. Drug Therapy 339: 1609-1618.
Osborne, M. R.; Davis, W. D.; Hewer, A. J.; Hardcastle, I. R.; Phillips, D. H. (1999) 4-Hydroxytamoxifen gives DNA adducts by chemical activation, but not in rat liver cells. Chem. Res. Toxicol. 12: 151-158.
Oscarson, M.; Hidestrand, M.; Johansson, I.; Ingelman-Sundberg, M. (1997) A combination of mutations in the CYP2D6*17 (CYP2D6Z) allele causes alterations in enzyme function. Mol. Pharmacol. 52: 1034-1040.
Paine, M. J. I.; Gilham, D.; Roberts, G. C. K.; Wolf, C. R. (1996) Functional high level expression of cytochrome P450 CYP2D6 using baculovirus expression systems. Arch. Biochem. Biophys. 328: 143-150.
Panserat, S.; Mura, C.; Gerard, N.; Vincent-Viry, M.; Galteau, M. M.; Jacqz-Aigrain, E.; Krishnamorthy, R. (1994) DNA haplotype-dependent differences in the amino acid sequence of debrisoquine 4-hydroxylase (CYP2D6): evidence for two major allozymes in extensive metabolisers. Hum. Genet. 94: 401-406.
Panserat, S.; Mura, C.; Gerard, N.; Vincent-Viry, M.; Galteau, M. M.; Jacqz-Aigrain, E.; Krishnamorthy, R. (1995) An unequal cross-over event within the CYP2D6 gene cluster generates a chimeric CYP2D71CYP2D6 gene which is associated with the poor metabolizer phenotype. Br. J. Clin. Pharmacol. 40: 361-367.
Parker, B. A. and Stark, F. R. (1979) Regulation of simian virus 40 transcription. J. Virol. 31: 360.
Pathak, D. N.; Pongracz, K.; Bodell, W. J. (1995) Microsomal and peroxidase activation of 4-hydroxy-tamoxifen to form DNA adducts: comparison with DNA adducts formed in Sprague-Dawley rats treated with tamoxifen. Carcinogenesis (Lond.) 16: 11-15.
Patten, C. J.; Smith, T. J.; Murphy, S. E.; Wang, M.-H.; Lee, J.; Tynes, R. E.; Koch, P.; Yang, C. S. (1996) Kinetic analysis of the activation of 4-(Methylnitroso)-1-(3-pyridyl)-1-butanone by heterologously expressed human P450 enzymes and the effect of P450-specific chemical inhibitors on this activation in human liver microsomes. Arch. Biochem. Biophys. 333: 127-138.
Penman, B. W.; Reece, J.; Smith, T.; Yang, C. S.; Gelboin, H. V.; Gonzalez, F. J.; Crespi, C. L. (1993) Characterization of a human cell line expressing high levels of cDNA-derived CYP2D6. Pharmacogenetics 3: 28-39.
Pierce, D. M.; Smith, S. E.; Franklin, R. A. (1987) The pharmacogenetics of indoramin in poor and excessive hydroxylators of debrisoquine. Eur. J. Clin. Pharmacol. 33: 59-65.
Poon, G. K.; Chui, Y. C.; McCague, R.; Lonning, P. E.; Feng, R.; Rowlands, M. G.; Jarman, M. (1993) Analysis of phase I and phase II metabolites of tamoxifen in breast cancer patients. Drug Metab. Dispos. 21: 1119-1124.
Poulos, T. L.; Finzel, B. C.; Howard, A. J. (1987) High-resolution crystal structure of cytochrome P450cam. J. Mol. Biol. 195: 687-700.
Price-Evans, D. A. P. (1993). Genetic factors in drug therapy. Clinical and molecular pharmacogenetics. Cambridge: Cambridge University Press.
Pritchard, M. P.; Glancey, M. J.; Blake, J. A. R.; Gilham, D. E.; Burchell, B.; Wolf, C. R.; Friedberg, T. (1998) Functional co-expression of CYP2D6 and human NADPH-cytochrome P450 reductase in Escherichia coli. Pharmacogenetics 8: 33-42.
Prueksaritanont, T.; Dwyer, L. M.; Cribb, A. E. (1995) (+)-Bufuralol 1′-hydroxylation activity in human and rhesus monkey intestine and liver. Biochem. Pharmacol. 50: 1521-1525.
Randerath, K.; Moorthy, B.; Mabon, N.; Sriram, P. (1994) Tamoxifen: evidence by ³²P-postlabeling and use of metabolic inhibitors for two distinct pathways leading to mouse hepatic DNA adduct formation and identification of 4-hydroxytamoxifen as a proximate metabolite. Carcinogenesis (Lond.) 15: 2987-2094.
Rauschenbach, R.; Gieschen, H.; Salomon, B.; Kraus, C.; Kuhne, G.; Hildebrand, M. (1997) Development of a V79 cell line expressing human cytochrome P450 2D6 and its application as a metabolic screening tool. Environm. Toxicol. Pharmacol. 3: 31-39.
Rettie, A. E.; Korzekwa, K. R.; Kunze, K. L.; Laurence, R. F.; Eddy, A. C.; Ayoma, T.; Gelboin, H. V.; Gonzalez, F. J.; Trager, W. F. (1992) Hydroxylation of warfarin by human cDNA expressed cytochrome P450: a role for P4502C9 in the etiology of (S)-warfarin drug interactions. Chem. Res. Toxicol. 5: 54-59.
Roh, H. K.; Dahl, M. L.; Johansson, I.; Ingelman-Sundberg, M.; Cha, Y. N.; Bertilsson, L. (1996) Debrisoquine and S-mephenytoin hydroxylation phenotypes and genotypes in a Korean population. Pharmacogenetics 6: 441-447.
Romkes-Sparks, M.; Mnuskin, A.; Chem, H. D.; Persad, R.; Fleming, C.; Sibley, G. N.; Smith, P.; Wilkinson, G. R.; Branch, R. A. (1994) Correlation of polymorphic expression of CYP2D6 mRNA in bladder mucosa and tumor tissue to in vivo debrisoquine hydroxylase activity. Cacinogenesis 15: 1955-1961.
Roy, S. D.; Hawes, E. M.; McKay, G.; Korchinski, E. D.; Midha, K. K. (1985) Metabolism of methoxyphenamine in extensive and poor metabolisers of debrisoquine. Clin. Pharmacol. Ther. 38: 128-133.
Sabbagh, N.; Brice, A.; Marez, D.; Durr, A.; Legrand, M.; Lo Guidice, J.-M.; Korzekwa, K. R.; Destee, A.; Agid, Y.; Broly, F. (1997) CYP2D6 polymorphism in familial and sporadic Parkinson's disease. unveröffentlicht.
Sabbagh, N.; Brice, A.; Marez, D.; Durr, A.; Legrand, M.; Lo Guidice, J.-M.; Korzekwa, K. R.; Destee, A.; Agid, Y.; Broly, F. (1999) CYP2D6 polymorphism and Parkinson's disease susceptibility. Mov. Disord. 14: 230-236.
Sachse, C.; Brockmöller, J.; Bauer, S.; Reum, T.; Roots, I. (1996) A rare insertion of T226 in exon 1 of CYP2D6 causes a frameshift and is associated with the poor metabolizer phenotype: CYP2D6*15. Pharmacogenetics 6: 269-272.
Sachse, C.; Brockmöller, J.; Bauer, S.; Roots, I. (1997) Cytochrome P450 2D6 variants in a Caucasian population: allele frequencies and phenotypic consequences. Am. J. Hum. Genet. 60: 284-95.
Saiki, R. K.; Gelfand, D. H.; Stoffel, S.; Scharf, S. J.; Higuchi, R.; Horn, G. T.; Mullis, K. B.; Erlich, H. A. (1988) Primer-directed enzymatic amplification of DNA with a thermostable DNA polymerase. Science 239: 487-491.
Sambrook, J.; Fritsch, E. F.; Maniatis, T. (1989). Molecular Cloning. 2nd Edition. Cold Spring Harbor, N.Y.: Cold Spring Harbor Laboratory Press.
Sawada, M. and Karnataki, T. (1998) Genetically engineered cells stably expressing cytochrome P450 and their application to mutagen assays. Mutation Research 411: 19-43.
Saxena, R.; Shaw, G. L.; Relling, M. V.; Frame, J. N.; Moir, D. T.; Evans, W. E.; Caporaso, N.; Weiffenbach, B. (1994) Identification of a new variant CYP2D6 single base pair deletion in exon 3 and its association with the poor metabolizer phenotype. Hum. Mol. Genet. 3: 923-926.
Schenkman, J. B. und Greim, H. (1993). Cytochrome P450, 105. Berlin: Springer.
Schenkman, J. B.; Tamburini, P. P.; Jansson, I.; Epstein, P. M. (1989). Functional aspects of protein-protein interactions of cytochrome b₅, cytochrome P-450 reductase and cytochrome P-450. In: Book of abstracts and lecture notes. NATO Advanced Study Institute on Molecular Aspects of Monooxygenases and Bioactivation of Toxic Compounds.
Schmalix, W. A.; Barrenscheen, M.; Landsiedel, R.; Janzowski, C.; Eisenbrand, G.; Gonzalez, F.; Eliasson, E.; Ingelman-Sundberg, M.; Perchermeier, M.; Greim, H.; Doehmer, J. (1995) Stable expression of human cytochrome P450 2E1 in V79 Chinese hamster cells. Eur. J. Pharmacol. 293: 123-1131.
Schmalix, W. A.; Maeser, H.; Kiefer, F.; Reen, R.; Wiebel, F. J.; Gonzalez, F. J.; Seidel, A.; Glatt, H. R.; Doehmer, J. (1993) Stable expression of human cytochrome P450 1A1 cDNA in V79 Chinese hamster cells and metabolic activation of benzo[a]pyrene. Eur. J. Pharmacol. 248: 251-261.
Schneider, A.; Schmalix, W. A.; Siruguri, V.; de Groene, E. M.; Horbach, G. J.; Kleingeist, B.; Lang, D.; Bocker, R.; Belloc, C.; Beaune, P.; Greim, H.; Doehmer, J. (1996) Stable expression of human cytochrome P450 3A4 in conjunction with human NADPH-cytochrome P450 oxidoreductase in V79 hamster cells. Arch. Biochem. Biophys. 332: 295-304.
Schulz, M.; Freisem-Rabien, U.; Jessberger, R.; Doerfler, W. (1987) Transcriptional activities of mammalian genomes at active sites of recombination with foreign DNA. J. Virol. 61: 344-353.
Schulz-Utermoehl, T.; Bennett, A.; Ellis, S.; Tucker, G. B. A.; Edwards, R. (1999) Polymorphic debrisoquine 4-hydroxylase activity in the rat is due to differences in CYP2D2 expression. Pharmacogenetics in press.
Sengstag, C.; Eugster, H. P.; Wurgler, F. E. (1994) High promutagen activating capacity of yeast microsomes containing human cytochrome P-450 1A and human NADPH-cytochrome P-450 reductase. Carcinogenesis 15: 837-843.
Sharp, P. A.; Sugden, B.; Sambrook, J. (1973) Detection of two restriction endonuclease activities in Haemophilus parainfluenzae using analytical agarose-ethidium bromide electrophoresis. Biochemistry 12: 3055-3063.
Shimada, T.; Yamazaki, H.; Mimura, M.; Inui, Y.; Guengerich, F. P. (1994) Interindividual variations in human liver cytochrome P-450 enzymes involved in the oxidation of drugs, carcinogens and toxic chemicals: studies with liver microsomes of 30 Japanese and 30 Caucasians. J. Pharmacol. Exp. Ther. 270: 414-423.
Simi, S.; Xiao, Y.; Campagna, M.; Doehmer, J.; Darroudi, F. (1999) Dual-colour FISH analysis to characterize a marker chromosome in cytochrome P450 2B1 recombinant V79 Chinese hamster cells. Mutagenesis 14: 101-105.
Smith, D. A.; Abel, S. M.; Hyland, R.; Jones, B. C. (1998) Human cytochrome P450s: selectivity and measurement in vivo. Xenobiotica 28: 1095-1128.
Smith, L. L. and White, I. N. H. (1998) Antiestrogen therapy: uncertainties and risk assessment. Oncology 12 Suppl. 5: 14-22.
Stearns, V. and Gelmann, E. P. (1998) Does Tamoxifen cause cancer in humans? J. Clin. Oncol. 16: 779-792.
Steen, V. M.; Molven, A.; Aarskog, N. K.; Gulbrandsen, A.-K. (1995) Homologous unequal cross-over involving a 2.8 kb direct repeat as a mechanism for the generation of allelic variants of the human cytochrome P450 CYP2D6 gene. Hum. Mol. Genet. 4: 2251-2257.
Steiner, E.; Iselius, L.; Alvan, G.; Lindsten, J.; Sjoqvist, F. (1985) A family study of genetic and environmental factors determining polymorphic hydroxylation of debrisoquin. Clin. Pharmacol. Ther. 38: 394-401.
Strain, A. J. and Wylie, A. H. (1984) Uptake and stability of SV40 DNA after calcium phosphate transfection of CV-1 cells. Biochem. J. 218: 475-482.
Styles, J. A.; Davies, A.; Lim, C. K.; De Matteis, F.; Stanley, L. A.; White, I. N. H.; Yuan, Z.-X.; Smith, L. L. (1994) Genotoxicity of tamoxifen, tamoxifen epoxide and toremifene in human lymphoblastoid cells containing human cytochrome P450s. Carcinogenesis 15: 5-9.
Swierenga, S. H.; Heddle, J. A.; Sigal, E. A.; Gilman, J. P.; Brillinger, R. L.; Douglas, G. R.; Nestmann, E. R. (1991) Recommended protocols based on a survey of current practice in genotoxicity testing laboratories, IV. Chromosome aberration and sister-chromatid exchange in Chinese hamster ovary, V79 Chinese hamster lung and human lymphocyte cultures. Mutat. Res. 246: 301-322.
Szczesna-Skorupa, E.; Straub, P.; Kemper, B. (1993) Deletion of a conserved tetrapeptide, PPGP, in P450 2C2 results in loss of enzymatic activity without a change in its cellular location. Arch. Biochem. Biophys. 304: 170-175.
Tenni, P.; Lalaich, D. L.; Byrne, M. J. (1989) Life threatening interaction between tamoxifen and warfarin. Br. Med. J. 298: 93.
Tucker, G. T. (1998). Clinical aspects of polymorphic drug metabolism. In: Drug metabolism. towards the next millenium, Gooderman, N. (ed.). Amsterdam, Berlin, Oxford, Tokyo, Washington, D.C.: IOS Press, 109-118.
Tucker, G. T.; Lennard, M. S.; Ellis, S. W.; Woods, H. F.; Cho, A. K.; Lin, L. Y.; Hiratsuka, A.; Schmitz, D. A.; Chu, T. Y. (1994) The demethylenation of methylenedioxymethamphetamine (“ecstasy”) by debrisoquine hydroxylase (CYP2D6). Biochem. Pharmacol. 47: 1151-1156.
Tyndale, R.; Aoyama, T.; Broly, F.; Matsunaga, T.; Inaba, T.; Kalow, W.; Gelboin, H. V.; Meyer, U. A.; Gonzalez, F. J. (1991a) Identification of a new CYP2D6 allele lacking the codon encoding Lys-281: possible association with the poor metabolizer phenotype. Pharmacogenetics 1: 26-32.
Tyndale, R. F.; Kalow, W.; Inaba, T. (1991b) Oxidation of reduced haloperidol to haloperidol: involvement of human P450IID6 (sparteine/debrisoquine monooxygenase). Br. J. Clin. Pharmacol. 31: 655-660.
Vorhees, C. V.; Reed, T. M.; Schilling, M. A.; Fisher, J. E.; Moran, M. S.; Cappon, G. D.; Nebert, D. W. (1998) CYP2D1 polymorphism in methamphetamine-treated rats: genetic differences in neonatal mortality and effects on spatial learning and acoustic startle. Neurotoxicology and Teratology 20: 263-273.
Wadelius, M.; Darj, E.; Frenne, G.; Rane, A. (1997) Induction of CYP2D6 in pregnancy. Clin. Pharmacol. Ther. 62: 400-407.
Wahl, G. M.; de Saint Vincent, B. R.; DeRose, M. L. (1984) Effect of chromosomal position on amplification of transfected genes in animal cells. Nature 307: 516-520.
Wang, S. (1992). Phenotypes and genotypes of debrisoquine hydroxylation polymorphism in Chinese. Master's thesis. National Cheng Kung University, Tainan, Taiwan.
Wilking, N.; Isaksson, E.; von Schoultz, A. (1997) Tamoxifen and secondary tumors. Drug Safety 16: 104-117.
Wolfel, C.; Platt, K. L.; Dogra, S.; Glatt, H.; Wachter, F.; Doehmer, J. (1991) Stable expression of rat cytochrome P450IA2 cDNA and hydroxylation of 17 beta-estradiol and 2-aminofluorene in V79 Chinese hamster cells. Mol. Carcinog. 4: 489-498.
Yamamoto, Y.; Tasaki, T.; Nakamura, A.; Iwata, K.; Kazusaka, A.; Gonzalez, F. J.; Fujita, S. (1998) Molecular basis of the Dark Agouti rat drug oxidation polymorphism: importance of CYP2D1 and CYP2D2. Pharmacogenetics 8: 73-82.
Yokota, H.; Tamura, S.; Furuya, H.; Kimura, S.; Watanabe, M.; Kanazawa, I.; Kondo, I.; Gonzalez, F. J. (1993) Evidence for a new variant allele CYP2D6J in a Japanese population associated with lower in vivo rates of sparteine metabolism. Pharmacogenetics 3: 256-263.
Zanger, U. M.; Vilbois, F.; Hardwick, J. P.; Meyer, U. A. (1988) Absence of hepatic cytochrome P450bufI causes genetically deficient debrisoquine oxidation in man. Biochemistry 27: 5447-5454.

1. Test system consisting of cells expressing a cytochrome P450 2D6 (hCYP2D6) allele in a heterologous manner wherein at least three P450 2D6 alleles are expressed in said test system.

2. Test system according to claim 1 wherein said at least three P450 2D6 alleles correspond to the most frequent allele types in a population.

3. Test system according to claim 1 wherein said test system expresses at least 5 functional hCYP2D6 alleles in a heterologous manner.

4. Test system according to claim 3 wherein the alleles hCYP2D6*1, *2, *9, *10 and *17 are expressed.

5. Test system according to any one of claims 1 to 4 wherein said cells are Chinese hamster lung fibroblasts or cells derived therefrom.

6. Test system according to claim 5 wherein said cells are V79 cells.

7. Test system according to claim 6 wherein said cells are the cell lines V79MZh2D6*1, V79MZh2D6*2, V79MZh2D6*9, V79MZh2D6*10 and V79MZh2D6*17 deposited on Feb. 15, 2000, at the DSMZ-Deutsche Sammlung von Mikroorganismen und Zellkulturen GmbH under the accession numbers DSM ACC2446, DSM ACC2447, DSM ACC2448, DSM ACC2449 and DSM ACC2450.

8. Test system according to any of the claims 1 to 7 wherein said cells express cDNA.

9. Kit comprising the test system according to any of the claims 1 to 8.

10. Use of the test system according to any of the claims 1 to 8 for the study of the gene-dependent toxicity of metabolites.

11. Use according to claim 10 wherein said metabolites are drugs.

12. Use of the test system according to any of the claims 1 to 8 for determining a toxic, mutagenic or cancerogenous effect of compounds.

13. Use according to any one of claims 10 to 12 wherein the cells expressing human cytochrome P450 2D6 are contacted with the substance to be tested.

14. Method for screening of substances with respect to their metabolization by human cytochrome P450 2D6 wherein the cells of the test system according to any of the claims 1 to 8 are contacted with a substance and the metabolic product is measured.

15. Method for the detection of novel P450 2D6 alleles wherein said method comprises the heterologous expression of the allele in question in a cell, testing the cells expressing the allele in question with respect to the cytochrome P450 2D6-dependent metabolism of one or more compounds and comparison of the metabolism of the cells to the metabolism of cells of the test system according to any one of claims 1 to 8.

16. Method for the quantification of the cytochrome P450 content wherein said method comprises the solubilization of cytochrome P450 by means of the non-ionic detergent emulgen 913, centrifuging the solubilizate and measurement using CO difference spectra.

17. Method according to claim 16 wherein said method comprises the following steps:
(a) preparation of cell homogenate;
(b) addition of emulgen 913 to the cell homogenate;
(c) removing insoluble material;
(d) determination of the reduced spectrum;
(e) saturation with carbon monoxide;
(f) measurement of the CO/reduced spectrum;
(g) evaluation of the cytochrome P450 content by means of the spectra.

18. Method according to claim 16 or 17 wherein emulgen 913 is added in a final concentration of 0.25% (w/v).


SEQUENCE LISTING (19 sequences)

SEQ ID NO: 1 - DNA, 22 bp, Artificial
  Synthetic nucleotide
cgcaactggtcgaagcagtatg

SEQ ID NO: 2 - DNA, 18 bp, Artificial
  Synthetic oligonucleotide
agctggatgagctgctaa

SEQ ID NO: 3 - DNA, 19 bp, Artificial
  Synthetic oligonucleotide
atcaccaacctgtcatcgg

SEQ ID NO: 4 - DNA, 20 bp, Artificial
  Synthetic oligonucleotide
ggaatgtcctctcaagtaga

SEQ ID NO: 5 - DNA, 20 bp, Artificial
  Synthetic oligonucleotide
cagaggttttcaccgtcatc

SEQ ID NO: 6 - DNA, 22 bp, Artificial
  Synthetic oligonucleotide
catactgcttcgaccagttgcg

SEQ ID NO: 7 - DNA, 31 bp, Artificial
  Synthetic oligonucleotide
agacgtgaagcttgccgccaccatggggcta

SEQ ID NO: 8 - DNA, 32 bp, Artificial
  Synthetic oligonucleotide
caggacgtagaatggatctggatgatgggcac

SEQ ID NO: 9 - DNA, 20 bp, Artificial
  Synthetic oligonucleotide
tagacaagcttggatccatg

SEQ ID NO: 10 - DNA, 20 bp, Artificial
  Synthetic oligonucleotide
gctataagcttagatctcgg

SEQ ID NO: 11 - DNA, 84 bp, Artificial
  Synthetic oligonucleotide
tagacaagcttggatccatggtaccgagctcgagtcgactgcagttaactctagatcgatgcggccgagatctaagcttatagc

SEQ ID NO: 12 - DNA, 21 bp, Artificial
  Synthetic oligonucleotide
gcaggtgagggaggcgatcac

SEQ ID NO: 13 - DNA, 20 bp, Artificial
  Synthetic oligonucleotide
cactgcttactggcttatcg

SEQ ID NO: 14 - DNA, 20 bp, Artificial
  Synthetic oligonucleotide
actagaaggcacagtcgagg

SEQ ID NO: 15 - DNA, 20 bp, Artificial
  Synthetic sequence
gcattaagcttaagtcgacc

SEQ ID NO: 16 - DNA, 21 bp, Artificial
  Synthetic oligonucleotide
ccgtatgatcactagtagatc

SEQ ID NO: 17 - DNA, 114 bp, Artificial
  Synthetic oligonucleotide
gcattaagcttaagtcgaccggtaccgtacgctagcgaattccggatatcgatggcgcgccgcggccgctcgagctctagacgcgtggatccagatctactagtgatcatacgg

SEQ ID NO: 18 - DNA, 19 bp, Artificial
  Synthetic oligonucleotide
tattccagaagtagtgagg

SEQ ID NO: 19 - DNA, 18 bp, Artificial
  Synthetic oligonucleotide
atcaccgagctgagaagc